SEQUENCE LISTING (202 sequences)

SEQ ID NO: 1 - DNA, 3267 bp, H. Sapiens
ggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaagtacgtgagccatctccctgcctgctagagggacccagcctcgggggtggagggagcattccaccctgcttagatcccacatccagcttgccatcaacacagcctagggttgtggccaccccgcatcccccagatggtcctctggccaccagacgcctgtagacagaggttctaggagggggtgacagtcttgccaactaatacctccccctatccctttggttccacatccaagggtagggccctgaccccaggaagtgtctgtctctctctctctgtctcccacccagctgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtcaaaaaaaaaaaaaaa

SEQ ID NO: 2 - DNA, 781 bp, H. Sapiens
gcccaagtggaagaatttcaaaggcctcgagctgctctgcagtagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcgggtcagtgggggagggccaggaggccccagagctttcctcctgcggctgccggctaccgcccgcctcggaagatccctggaaaggggatcctgacccccgagcttgcgcccctcgggccctccattcagtcccgggccgacagcgccaccgtgtggccacagcgtctcctagcggcctccttacctaggggtcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcggacactctcttcaccatgactcagtccggccttcgcccctgcagctgccgctgaggatgctacgtcggcatgctgacatgatccagccggactgacgcactgcagcaagctggatgacttcatggacatctcaga

SEQ ID NO: 3 - DNA, 695 bp, H. Sapiens
agcgggttccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatttctttaccaactcccgcctcccacagccgcccatgccttcaaacttcccagagccccccagcttcagccccgtggttgactccctcttcagcagtgggacactgggcccagaggtgcccccagcttcctcggccatgacccacctctctggacacagccgtctgcaggttcggaacagctgccctggccccttggactccagcgccttcctgagttctgatttcctccttcctgaa

SEQ ID NO: 4 - DNA, 330 bp, H. Sapiens
gggaagaggaggaagaagaaggggactgggtctggggccagatgagaggaagggggcaatggggaccctttcccctgggtcggtctttctgtgggtctgtctgtccctaccacaggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactc

SEQ ID NO: 5 - DNA, 3293 bp, H. Sapiens
ccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttatctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggttaaaaaaaaaaaaaaa

SEQ ID NO: 6 - DNA, 3236 bp, H. Sapiens
ccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttatctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggttaaaaaaaaaaaaaaa

SEQ ID NO: 7 - DNA, 3287 bp, H. Sapiens
ccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttatctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggttaaaaaaaaaaaaaaa

SEQ ID NO: 8 - skipped

SEQ ID NO: 9 - DNA, 3142 bp, H. Sapiens
ccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttatctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggttaaaaaaaaaaaaaaa

SEQ ID NO: 10 - DNA, 32147 bp, H. Sapiens
tacaggctttctccactatgtccggctaatttttgcatttttttggtagagatgggggtcttgccatattgctcaggctggtctcgaactcctgacctcaagccatcctcccgccttggcctcctaaagtgctaggattacaggcaagagcccctggcgtccggcctgacctttcttaatatccctttggagcaaagtaggggcggctctatcaggactccaaggaaagacgggaagggagatggggccaacccgccagagctccagagcaccgggttctcacctggcgtaaggattatgctaatacaagccccgccccgtcccaggccccgcccacccgatcggcctatggtgtgggggcggggctatgttaatgaggcccggctggccaagagctgttccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacaggtgagggcggttccgggccctgacggccttggcttccaggtggggatggggggggggtccggggcaggactgcgcgctggggccctcaacaccccacccgagcccggtcctgcggctcccggaggctccgatcacgccctggacaccccctttctcccagccccgaggtcctcctacagcggcccagcccgctggcccctgacggacccacgcatttctgattcccattcctcccccattccctcttccccgaagcgcagaccgcctgcgccccacctctacctggacctctggtcccctctctctggtcccagcccctccgtccctacaaaaatgccaggcgcccctggtccctaggccacccgcgcccatccccactctccactctggagccactccacccgaccccctgcaggcctcggccttctccccagcagccccggcccaagcccgccctattcctggaccctgagatactcccagcgcccctgattgtcccagttcataaccaaagtggactggaactggcccactctcacgtccacctccccgcaattgctgagacacctcttcttgatctgctacattaagggtggcgggaggaggatgtcccagaaatgtccaagaggcgcagctcgcagaggttacgccggtgcccgggaggcctgggttcgcctacccacgtcgccacactaggctgagcgcccgtcgctgcctgggacccccgcttagtgccaccgagagcgcacaggccgcgccccgctgccgctccacgccccgcgtcccagaggagggcgcgaacccgaggcctggcccagtgccctggagcaaagttccccggagttgaggggccagtataaattatcaaccgaaaacccgtaggggagaaagggggatcggccggccttgatcttttctctgtgcaaatattcctcttcctccgcggcgcctcctccccttctgagaaccgctggctacagatcggcacctgccgccctgcgggggagcaggagctggggtggggactgtgaggggagtgttgcctgcccccaaccgcagccgcccagctgcgaccccattggctggaggcggagtaaccttggcccacaagctctggcccctggcccaggtcatgttgcaacccgatcggccagggcgtcggacccgtgtggaaagttcaaattcccgtcccttctccctccctccccaaatgtcacgcgactgcctgggacagggcatctaaggtcctggtacaccggatgccggcactcctcctcctaagggtctcccatttcccacccaaccccaactttggctgctcattatatactacacatcactctatttgggagggggcctcacttccaaccctgacgatgggtgaccaaagaggagaggttctggggagctggcaaggtaagggacagagcgcacccccaaaattgcccccccacccagaggctgactcaaggctgatgtaaccccagtcagagatagctggggtagagaggactgtgttttcttcccaaatacccaaatattaatagtaaatctctggctgggcacagtggctcctgcctgtaagcccagcactttaggaggccaaggttctcatccaaggattgcttgagcccaggagttggagaccagcctgggcaacatactgagaacctgtctctaccaaaaaaaaaaaaatttttttttttttttgagacagagttttgctcttgttgcccaggctggagtgcagtggtgccatctcagctcactgcaacctctgccttccgggttcaagagaatctcctgcctcagcctcctgagtagctgggattacaggcgcccaccacctccagctaattttttgtatttttagtagagacgagatttcaccatgttggccaggctggtctcaaactcctgacctcaggtgatccacctgccttggcctccccaagtgctgggattacaggcatgagccaacatgcctggccaaaaaaaattttctttaatcagccaggtgtgatggcacattcctagtaccagctactggggagtctgaggtgggaggatcacttcagcccaggaggttgaggctccagtaagacatgatggtgccactgcactccaccctgggtgatagagtgagaccttgttggaaggaagggagggagggagggagggagatggggggaggaaggaaggaagggagggagggagatggaaggagggagggagataaaggagggagggagatggagagagggagatggagagagggagatggagggagggagggagatagagggaaatggagggagggagatggaggagggagggagaaggagggagggagggagatggagggagggagacggagggagatggagggagggagagggaaggagatggagggagggaggaagggagagagggaggaagggagggagggagggagatggagggagggagggagctggagagagatggagggagggagggagatggagggagggagggagatggagggagatggagggttggctcggtaccaggagagcttctagatttccccatttggtagtggaaaagccctgaggcttgaactgggacagtgttttcatcctcaaagacccccagttccaggggtcccctggaggggagcagattacaggagctgagtgtcccaggggagtgactatatgggaagagacagtaaggcagagagttcagaaggtggtgggaagaacagcgagcttgaccagggacccacctgaatcagggaccagcctgacccagagaccagcctgcagaggcctggcaaggtttcaccctctgcaggttagcgtgggcagagggaggatcgaggaggccgtcttccatggctccgtgggtggaagccccagggaggtagtttctggaagcttcaaggaagcacttgttcatggtcagagctggctagctttaggaagagtggccttgagggtgcaggggagcaaacaggacctggttagggggcaatctaaaaggaattcctgaaaggtgggaggtgggcagctgaacttggggttttctcaaccccctctctaagtctcagagttttcttctccttcttattatttatttatttatttatttatttatttatttttggagacagagtctcactctgttgcccaggctggagtgcgctggcatgatctcagctcactgtaatctccgtctcctgagttcaagcgattctcatgtctcagcctccagagtagctgaggtaacaggcatgtgccaccatccccagctaatttttaattttcagtagagacgaggtttcaccatgttggccaggctggtctcgaacccctgacctcaagtgatctgcccacctcagcctcccaaagtgctgggattacaggtatgagccatcgtgtcctgcctttttctttcttttcttttttttttctgagacggagtctcactctgtcgcccaggctggagtgcagtggcgcaatctcagctcactgcaacctccacccaacgggttcaagcaattctctgcctcagcctcctgagtagctgggattacaggcgcctgccaccacatccggctttttttttttttttttttcagtacaggtggagtttcaccatgttggccaggctggtcttgaactcctgaccttgtgatccacccgtctcggcctcccaaagtgctgggattacaggtgtgagctactgggcccagctcttttcttcttttaatagtttttgttgttgttgttgttgttttttgtttgttgttgagacagagtctcgctgtgacactcaagctggagtacaatggcatgatctcggctcactgcaacatccgcctcctgggttcaagcgattctcctgcctcagcctcccgagtagttgggattacaggcacgcaccaccatgcccggctaatttttgtatttttagtagagacggggtttctccatgttagccaggctggtcttgaactcctgacctcaggtgattcgcctgccttggcctcccaaagtgctgggattatagacgtgagccaccgcgcctggccctattaatagttttaaatagagataaggtctcactgtattgcccagctggtcttgaactcctgggttcaagcgatcctctcccctcagcctcccaaagtgcttaggttacaggcccaccactgctcctcatcagacttttctattggtcataacaagtttttgttttgttttgttttgtttgtttgttttttgagacggagtcttgctctgtcgcccaggctggagtgcagtggcgcgatcttggctcactgcaagctctgcctcccgggttcacaccattctcctgcctcagcctcccgagtagctgggaccacaggcacctgccaccacgcccagctaattttttgtatttttagtagagaccgggtttcactgcgttagccaggatggtctcgatctcctgaccttgtgatccacccatctcggcctcccaaagtgctgggattacaggcttgagccactgcgcccaaccagaatttacttgttttttatgagtcgggttcccactctgttgcccaggctggagtgcagtggtgtgatcacagctcacttcagccttgaactcctgggctcaagcgatcttcccactgcagcctcctgagtagctgagactagaggtttgcaccaccatgcccagctaacttttttttttttttttttgagatggagtcgctctctgtcgcccaggctggagtgcagtggcacaatctcggctcactgcaaactccgcctcccgggttaacgccattctcctggcttagcctcccaagtagctgggactacagacgcctgccaccacgtccggctaatttttgtatttttagtagagacggggtttcacgttgttagccaggctgatctcgaactcctgacctcaggtgatccacccacctcggcctcccaaagtgctgggattacaggcatgagccactgtgcctggccccagctaactttttaactgtagaaacagggttgcactatgttgcccatgctgatctcaaactcctggcctcctacagtcctcctgcctcggccttccaaagtgctgggattacaggtgtgagccatccccagccagtcataaaaagttcttgaaccctagcgttacgctcttcctccaggctgagtctcttctctcacagccccaaatccttttcttcctcctgggcttggatattgtcactgtcacatctgctctaaccctgcctgggccatgaacgtcataaccttagcatggaagcgggagagaaggcaggcaggtcaagggtgcgacacggggtgatcagaatgggctgggaggttgctgggatacagacagggacaacagactcaggagtcccccaccaggtgccacagaagaagctggcccaggaggtagcagagtctccccccacccccacctccatgtctccctaccaaacagatggtgtttatctaattctcagagggaggggcccagccagtggcccagagagctgggctgtgtatcttatttgggtaatgaggcctgttgtccctggtggggagggggcagtcccccacccccaccccaaagcaaacacaggaacttggagaccagggaggagagagggcccaatgaccaatgcactcaagtagggagaggctgcggagtcagactggcacccacccacatttcacccccagaatgtaggaaagacccctggaggaagcaggtatggttttctgaaagactaagccttgggggccaggtgcggtggctcacgcctgtcatcccagcactttgggaggccaaggtgggtggatcacttgaggtcaggagttcgagaccagcctggccaacatgatgaagccccgtctctactaaaaatacaaaagtgagctgggtgtcatggcatatgcctgtaatcccagctactcagggggctgaggcacgagaatcatttgaacctgggagacggaagttgcagtgagccaagatcatgccactgcactccaacctgggtggcagaatgaggctccatctcaaaaaaatgaaaaaatggctaagacttggggcctcagccttcccatctgagatctgagtaagggatgcaaaaggcgggctagaggggactggtacacaaccctggcttgtggatggagacgagagagggagggagagactagcagggggtctaagaccctgaatgtcttggcctcctggctcagcttcctggaggcagaagacttggcctgtgcctagcaggaccctcaccagtgtaagctcttccatccagtggatttttttttccttcaagacaggatcttgctctgttgcccaggctggagtgcagtggcgtgatcatagctcactgtagcctcaaactcccgggctgaagaaatcctctgacctcgcctccctagtagctaggactataggtgcatgccaccacacccggctaattttaaaattattttgtagagacagggtcttgctatgttgcccaggctggtctcaaactccttggctcaagcgatcctcccgccttggcctcccaaagtgttgggattacaaccgtgagctaccgcgcccagcccatcctgtggatttgatggacagctagggttagagtgagggtgctctgtctccatgaataccatttgctcatcctagcggccttcctttgggatctgttggttcattgtcttctgtgcagccctgagaagagcacaccttctcgctgagtccaggttgccaccctgggattctgggcatggcagacaccaaaaggactgtaccatgaaggcagtgactgccactctctataaagaggcaccctcggctgggcatggtggctcacatctgtaatcccagcactttgggaggccaaggtgggcagatcacttgaggtcaggagtttgagaccagcctgggcaacatggtgaaaccccgtctttactaaaaataccaaaaaaattagctgaacgtggtggcgcacaactgtaatcccagctactcgggaggccgaggcacgagaattgtttgaatccaagagatggaggttgtggtgagctgagatctcaccactgcactccagcctggatgacagactgagactccgtctcaaaaccaaaagcaaaccaaaaaaaaccaaaccaaaccaaaacaaaacacagaggctccctctctaccaggcaaaccatggccatcccctttgcctctcctgggcctaaggaggcctgagtgacctcggacctgcccctttcctccctgactctcaggaaggtcttttttttttttttttttttttgagacggagtttcactcttgttgcccaggctggagtacaacggtgtgatctcagctcactgcaacctccgcctcccaggctcaagcaattctcctgcttcagcctcctgagtagctgggattacaggtgtgcgccaccacagccagctaattttgtatttttagtagagacagggcttcagtatgttggactaggctggtctcgaactcctgacctcaggcgatccacccgccttggccccgcaaagttctgggatgacaggcgtgagctactgcacccagcccaggaaggtcatttcttattctcttaaatgagaaatgggcctgagcactgtccccagccagcctccaagacacacctgtgaggccactggacattgttgcttccagggccgagctgatccacaggtttcagcgtggcttttgttctgcagtagactgacaaacaggtctccccagggaacacaggggccgctggtgggagtctggaaggggattttgaaggtctgactctctggataggaatttcaggcaggttgggttggggggccagatttggaggggcccctcaaatgccaaccatagatggatgcctagtgcatggggaatatggggaccagcctctgtggcagcactgcatctccctaaccctactcctctcccctttgacagtggcaagctggtgtctcccaagtggaagaatttcaaaggcctcaagctgctctgcagagacaagatccgcctgaacaacgccatctggagggcctggtatatccagtgtgagtggctaccaccagcctcccaagccgggagggactgccccctccaaccctcctgccagagggttctacatggtggctgcccaggaaaatccccggggtcctcaaggccagcactagcagagttctgccaaccttttttttttttttttttttttgagacacagtcttgctctgttgcccaagctggagtgcagtggtgccatctcggctcactgcaacttccgcctcccaggttcaagccattctcctgcctcagcctcccaagtagctgggactgcaggcacccaccaccactcccagctaatttttgtatttttagtagagacggtttcaccatgttggccaggctggtcttgaactcctgacctcaagtgatctgcccaccttggcctcccaaagtgctgggattacaggcgtgagccaccacgcctgaccctgacagccttttagggggagaccaggcccccagcaccaggactcctgaatagctcctgacctccagcgggtctcggggaataggtcccaaagggtgtttccactgtgaagctgggcacagcagaggtcacccctcagtagcactagggtaggctgagatttgaggtacagttcaaagcccattttgcatcccattagcatagttgggtgccttgaatgcgagggtaaaaagggccattagacactagcggggccaactgcatcactttgtaaatggagaaactgaggatcccgcatgggtctccaccagcctttcattgaagtccttcctgaatatccagtaggtgcccaccatggccaattgggcagaggggatccagagaggccatgccatctcgtggaaggaactctggacttagagttgcccagagctagttcctccaccccgtctctgccccttgctggctgtgtgacttcaggctagttgcctagcctctctgaacttgtttccttatctataaaaacgaggccacggtggctcacacctgtaatcccagcactttggaaggccaaggcgggcagatcacaaggtcaggagatcgagaccatcctgcctaacgcagtgaaacccggtgtctactaaaaatacaaaaaattagctgggcgtggtggcgggtgcctgtagtcccagctactcggcaggctgaggcaggagaatggcgtgaacctgggaggcggagcttgcagtgagctgagatcgcgccgctgcactccagcctgggcgacagggcgagactccgtctcaaaaaaaaaaaaaaaaaaagggcaaaacaaaaaacgaggccaatagtcctaactttcatggttggtgtggagataacaggtattccagggtgttgccactaagttggtatccaatacatgatgtatccttaaggaggtcacattctcattcatttgggaacaccccactccccatggcagggcagcagaggcagagaggacttggatccgccagccctgtgttgggacctcattcaggtcaggttctgctgcaggatcaaaggctgcactgagctgggaatgcaaagcccaggagagaccacacgcagtattctctgtcctcgccccgctggatttcatccaagcagggagagggtgtgttttctggtttccatttatctacatgccaggcactttcgtgaatcaccttatttaaggcttcctagcaaccctgggagataggttttattaccgccatgttatggatgagcaactgaggctcagaaggcaagtgacatattcaggttcacagagcgtaaaagaagcagagctggggctggctggaagtccacagttccccaactccaaattatggcggtggggggggtttgtttggttttgttttgttttttaatacgaagtttcgctcatgttgcccatgctggagtgcagtggcgtgatcttggctcactgtaacctccgtctccctggtttaagagattctcctgcctcagcctcctgcacctctcctggggcaagaagaactttccactttgtattgtctcgtgttgtttgtttacttcccttatctctcccactaagctgtgggcaccaagtagctgggattacaggcacccgccaaattttgtatttttagtggagacagggtttcaccatgttgtccaggctggtctcgaactcctgacctcagatgatctgcccgcctcagcctcccaaagagctggggttataggcgtgagccaccacacccagcctgcagaagaaatttctgatggcagctgaggcatgatgtcacctggggtcctcacccagacacactgatctccagctattgcacaagttgctggaaggaccccaggttcggaatcagacagagtgaggtttcagtccagccctagcatgtcctaactgtgtgggcttgggaaattctctagcctcccggagtcgaagcttgctatttgtgaaaatgggaatgacaggtcctatttgttttttcttggttttcttttttttgaaacagtcttgctctgtcacccatgctgaagtgcaatggcacgatctcggctcactgcaaccttcacctcccagttcaagcaattattgtgcctcagcctcccaagtaactgggactacaggcgcatgccaccatgcctggctaatttttgtatttttagtagagacggggtttcaccatgttgcccaggctggtcttgaactcctggccacaagtgatctgcccacctcggcctcccaaagtgctgggattacaggcatgagccaccgcgcccggccaataggtcctatttctgaggttcactgagttttcagtgagatcatctgtatctcactgtcatagtcatagcagcacgtgacacacatacaacaaatgtcaagacccctcctcctccctaaacgccaaaggtttgaaggtgggcttcacgggagtggggaggagaagggcttacttggcttgacgccactaatgtcgggccttagggctcaagcaggaagggctctgccaccccctgcaggctcctatcctctaagattcttcctttcctgatccaacagtcatactcccacactctgaaatcgtcttggtgtcctgggagctccctggcactttgcaccgctcctggggcaagaaggactttccactttgcattgtctcgggttgtttgtttactttccttatctctcccgctaagctgtgggcaccctgcctgattcatccatgtgtcctgtcccagtatctgtctcagcataggtgcaaagggttccgcggctgagtgaagggaagaacttatcagtgttaaacaaagcctgccccagcccccaggaagccacatggttacagcctttccatgagaaacagtccagaggatggggtctctgggctggggaggggggcttcacttggccctgactttctgagagccctggacaaactttttttttttttttttgagatgggggtctcactctgttgctcaggctggagtgcaatggtgtgctctcgactcactgcagcctcctcctccggggttcaagcgattctcctgcctcaccctcctgagtagctgggactacaggtgcgcaccaccacgcccggctaattttttgtattttcggtagagacagggtttctccatgttggtcaggctggtctcgaactcccgacctcaggtgatccgcccacctcggcctcccaaagtgctgggattacaggcgtgagccaccgcgcctggcctaacaaacttaattttattctattttttgagacagggtctcacttcatcacccaggctggagtgcagtggagccaacatagctcactgcggcctccaattcctgggctcaagcaattcccccacctcggcctccctagtagctgggactacaggcatgcgcccccacacccagctattttttatttttaattttttgtaggggcagggtctcactaggttgctcaggctggtctcaaactcctggcctcaagcaatcctctcagttcagcctcccaaaatgttggggctacaggcgtgagccaccacaccgtaccaggacaaacttcatctttgctggtgttcatttcctcatctggaaagccaggggcagacacactttgagaccctccttccttatctagccccctgggagtctatgcacccaaggacccagccctttgttttctgtccaatctgggctcagaaaccaggacaaaacccctccttcaatcctatcagtgtccattttggagaatgctttattttattttatttttttttttttgagacagtctcactctgtcacccaggctggggtgcagtggtgtaatctcagctcactgcaacctctgcctcccgggttcaagcaattctcatgcctcagcctcccaagtagctgggattacaggcatgcgccaccacgcctggctaatttctgtatttttagtagagatggggtctcactatgttgcccatgctggtcttgaattcctagattcaagtgatacgccggccttagcctcccaaagtgctgagattacaggcgtgagccaccgtgcccagcagattttggagaatgttctttaggacagaactttgtaaatgatgctaaaaagccctagtaaatttcatgttccagtggtcccccttcctctgtgtcctcgtttgtcccagaggaagttggctggggcaggaagggtcagcagttgttctctttgtcagagcagccaggaaaagtctcaggttgcctctaagtagaccagctgtgtgtcaagctctgtgccaaaggcaggagctgcttttactccctgcagcaaggcgagcatgtaaagacctgacaggccagggtggaggggaggtgggcagcgctgtgggctttggcaggcaggaaggcttcctggaggaggcgctatcctgaattgtgggcagaggagggaaggtaagaggggcttccctgttagaataagaccagaagggccggatgtggtggttcacgcctgtaatcccagcactttaggaggccgaggcgagcagatcacttgaggtcaggagttcgagaccagccttgccaacatggagaaaccctgtctctactaaaaatacaaaaattagtggggcgtggtggcgctcacctgtaatcccagctactaggcaggctgaggcaggagaattgttcgaacccagaaggcaaaggttgtggtgagccaagatcaggccactgcactccagcctgggtgacagagtgagacttcatcttaaaaaaaaaaggaaaagaaaaggtgggctgtgtggcttcaaggtgaggtgtataaaggagacagggaggccgggcacagtgactcatgcctgtaattccagcattttgggaggccgagggaggtggatcgcctgagctcaggagttcgagaccagcctgggcaacatggcgaaaccctgtctctaccaaaaatacaaaaaaccagctgggcatggtagcacatgcctatagtcccagctactcgggaggctaaggtgggaggatcgcttgagcctgggaggtgcaggctgcagtgagccaaggccgtagcactgtaccactgctctccaacttgggtgacaagagtgagaccctgtctcaaaaaaaggagatgagatgctgagagaagcaagtggtggcccagtcatggaggagggggacgggtttggaagccaagccaaggagtgtggactttgtcccagaggtagtagggagccatggagggttccagacctgaactgcactttagaaagaattgtcctggctgcaactggaagatgagctccgagtgtgacactggagtcagggaggtccctgtgagaaatgacaccaggccaaggggtgacacagtgagagaggatggaggggaggggacagcgggagaaatgtcacagatcctatgagattgagatttttttctcaattagacccggaatataacagtcagacctggaatgctgggagtcagtggggtttttgtggcaagttttgggacatcctagggagggtccccaggcagctggatatgaggctagagcaggaaagggccagagtgagagaggctgcagggtgggcattgcaaaggagtcagtgaaaagtggtcaggggctgggagcggtggctcacgcctgcaatcccagcactttgggaggctgaggcagtcagatcgcttgagcccaggagtttgacaacagcctgggcaacatggtgaaaccatgttgaattttttttaaaaattcacagattagctgggtgcagtggtatacacctgtagtcccagctactctggaggctaaggtaggaggatcacttgcacccaggaggcggagatcgaagtacccaagatccctgccactgcactccagcctgggcaacagattgagaccttgtctcaaacacacacacacacactccatcctgggcaacagattgagtccttgtctcaaacacacacacaccagcctgggcaacagattcagtccttgtttcaagcatacacacacacacacacacacacacacacacacaaagcggccagggcccggtgaggtggctgacacctgtaatcccagtgctttgggagcctgagttgggggatcatttgaggtcaggagtttgagaccagcctgaccaatatggtgaaaccacgtctctattgaaaatacaaaaaattagctgggcgtggtaacgcgtgcctgtaattccagctacttgggaggctgaggcaggagaatcgcttgaacccgggaggtggaggttgcagtgagctgggatcgtggcactgcactctaggctgggcaacaagagtgaaacgccatctcaaaaaaaaaaaaaaaaaaaaaaaaagtggccagggaagctaggagataaagaaaagcctcgtgagcccaggaatttgaggccagtcctcaaagcaacatagtggggccctgtctctaccaaaaaattaaaaataacagtaataaaaaggaagaggccaggcacagtggctcacgcctgtaatcccaacaccttgggaggccaagacaagaggattccttgacctcaggagttcgagaccagcctgggcaacacagggagaccctgtctctacaaaaaatttaaaaattagccagacatggtggcgtacaccagaggctgaggcgggaggatcgcttgagcccaggaggtcaaggcttcagtgagctatgattgcgccactgcactctggcctgggcaacagagcaagaacctgtttcaaaacaacaacaaaaacaaaaaagagcctggagtgtgtgttggggccaagggcaagagcctggggagaggtcagaattagagactgggaggggaggtggaggcctggaagaggacacatgttggtggtcagaagtgaaccaagggtggttctctgcaatgaaagtgggtagtgcctagccttctgaggcagaggacccaaggctctaggcccctggcctgccagtctaaccaggactttctttttttcttttttttttttctttgagatggattcttgctctgtcaccaggctggaatgcagtggcgtgatctcactgcaacctccacctcccaggttcaagcgattctcctgcctcagcctcccaaggagctgggactataggcatgtgccgccatgctcggctaatttttgtatttttagtggagacggggtttcaccatgttggcaggctggtcttgatctcctggcctcgttattcacccacctaggcctcccaaagtgctgggattacaggtgtgagccaccgcacccagccctaaccaggactttctggaggcagctcactctctgtggcctatggccccacaatccattcagcagtctttgcaagtgtacctatggggctccagaggaacccagagaaggtgtgggaggttggattcacagtgccctgtagaggaagatcaggctgggtgaggtggctcaagcctgtaatcccagcactttggaagcccgaggcaggtggatcacctgaggtcagcagttcgagaccagcctggccaacatggtgaaaccccgtctctactaaaaacacaaaaattagctgggcctggtcgtgggcgcctgtaatcccagctactcaggaggctgaggcaggagaatcgcttgaacccaggaggtggaggttgcagtgagccaagatcacgccattgcactccagcctgggcaacaagagtgaaaccctatctccaaaaaaaaaaaaaaaaaaaggaagatctaggaggctagagcatcatccgagaagacttcctggaggaggcagcatgggcacttaatctgaacgtcaaaggccaagcaggatttcgcagggtgtcacttgagtcccagtcccctggggtgaggtgggggatgaggaagctgggtgcccctgacctggcccccttctctcagatgtgaagcggaggaagagccccgtgtgtggcttcgtgacccccctgcaggggcctgaggctgatgcgcaccggaagccggaggtagctgggttctggaccgcctgcccacctgcccacctgcccacccaccaagaccagcccctgcctcctgtagccttgcgctgagcagacagggatggggtgaagggtgggagatgggtgtgcggtgctcccctctctactgatccccctcccccggcccctaccccaggccgtggtcctggaggggaactactggaagcggcgcatcgaggtggtgatgcgggaataccacaagtggcgcatctactacaagaagcgggtcagtgggggagggccagggaggccccagagctttcctcctgcggctgccggctaccgcccgcctcggaagatccctggaaaggggatcctgacccccgagcttgcgcccctcgggccctccattcagtcccgggccgacagcgccaccgtgtggccacagcgtctcctagcggcctccttacctaggggtcgggtgagctcctgatgggaaatgggggatctcatcgcttgtgagtagaggagactttggggggaaagtgatggaggatggggcaagggatccggtgtccaactctgtgtgtccctgcagctccgtaagcccagcagggaagatgacctcctggcccctaagcaggtgggtgctctgtaagtccagcaggcaagggcatccctttgccccccaagtagatgggaggcagagttgacagtttccgcctcagtgggcaagacagtggctgggcatgagggtggaatgtgggatgttactgccatcctgtggttcatttgttgagctcttctcttcttggggggtgagggtgaggtggtgagaagaccccaggtcattgagcaagtggtgggaggcatgggagaacagagggaccaggccgggcgtggtggctcacgcctgtcatcccagcactttgggaggccgaggcgggtgggtcatctgaggtcaggagttcgagaccagcctggccaacatggtgaaaccccgtctctactaaaaatacaaaaattagccgggcatagtggcgcatgcctgcaatcccagctactcaggaggctgagacaggagaatcgcttgaacctgggaggtggaggttgcagtgagctgagattgcgccactgcactccagcctgggcaacagagtgagactccgtctccaaaaaaaaaaaaaagaaaaagaaaaagaaaacaaacaaaaaaaaaaaccagagggaccaaagaactggggtcccagacaggaaaaaatctaaagagcaggaaggggacccctgtatctgcatcctggtctatctcctgggggaagttcttagaggccaatggagtgatcttggcctttcaagcagcaagctaggtccactagggaaaggtgacagaggtgtgaacagacagaccctctgtcctgctgaccacttgaccctggagaggggtgatatggaagatcggggcagtgggagctctctagcggctgctgacggctctgtctccctaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatggtgagggtctgagaaagggcaggggagaggggtgaaggcctgtgagattccaggaggccaagggagggatggctggggatgggggtggggaccctgctcctccctgcctgctgctgctgatgtcgccactgctgcctccagcctacgtcggcaatgctgacatgatccagccggacctgacgccactgcagccaagcctggatgacttcatggacatctcaggtggggcgagcccctccccaggtcgggtggacgggcagggtctgtgtgaggaaggccgatgagcccatcccaggcccctctgggagggctgatagtctgggtcgtttctttttttctgtctttttatttatttatttattttgagatggcatctcactctgtcaccaaggttgcagtgcagtggtgccaacctctgccctccagactcaagtgatcctcccgcctcagcctcctgagtagctgggactacaggtgtgcaccaccaaacccggctaattttttttgtatttttggtagagatgggtttcaccatgttgcccagactggtcccaaactcctgagctcaagccatcctcttgccttggcctcccaaagtgctgggattacaggcatgagccaccatgcctggcaggtctgggtggtttctgagggttcttcctggtctccttttgtccagctggtgtggcctccaatgggggcagggcccaagagagtacctggccagggaaccaggggctcccaagaaagcagtgaaggctgggactctctcaggggagagaagaactttgaattatttgggtctatttatttttcacacaatgcattttaaaactatttgctaggctgggcatggtggctcacacctgtaatcccagcacttttgggaggccgagtcaggcagatcacctgacgtcaggagttcgagaccaacctgaccaatatggtgaaaccccatctctactagaagtaccaaaattagctgggcgtggtggcaggcacctgtgatcccagctactcaggaggctgaggtgggagaattgcttgaacctgagaggcagaggttgcagtgagccgagatcgcgccactgcactccagcctgggtgacagagcaagactctgtctcaaaaaaattaaaataaaaaataaaactatttactagagaacatttcaaacatgcacacactagtagagaaagagtagctcagcaaaaacctcccaagtcaccatcactgatgtcaccgacttatcaactcttggccagtcttttatccgagaacattccttctcttctgttatgtttctattttattttattttaatttaatttatttttgagacagggtctcattctgttgcccaggctggagtacagtggcacaatcacagctcactgcagcctcaacctcccaggctcaagtgatcctcccacctcagcctcccgagtagctgggaccacaggcacgcaccattatgcctggctaattttttttatgttttgaagagacagggcctccctgtattgcccaggctggtctagaactcctgggctcaagagatcctcccacctcagcctcccaaagtgctgggattacaggtgtgtgccaccgtgcccagtgcgttattttaaagaaaatcccaggcatgcatcatatcttgtaaagcttttttttttttttttttttttttttttgagacggagtctcactctgtcacccaggttggaatgcagtgagtccatctcagctcactgcagcttccgcctcccaggttcaagcgattcttgtgcctcagcctcctgagtagctgggattacaggcatgtgccaccacacccagctactttttgtatttttagtagagacggggttttgccatgttggccaggctggttttgaactcctgacctcaagcgatcctcccacctcggactcccaaagtgctgggattacgggtgtgagctaccgcgtctggcctaaagcaatttttgtaagtcatgagtgtccctgcctccctctcctgaccacagggccaccaagttactgattcctcagtgtcctcccagagatgctcctgtcagtagaagcacatgtggacaaatgtctctgttttcacaattgactttccaagtggccttcctcacttcgctgtaggtcttgcaggttattcccgatagccacacatttagggacccccggagctatgtgcgggccaggtagatatccaggctcagcccgtctcatcgaagcccctggggacagaaagagggtccctgggataaagcttagtcttagtgacctcctaattccttccccacggggtcctgggcctgtcaatcaagaggtggaggcctatggggcgggaactagggaatgaggaaccacccccacccctgttccagtcccgggagggggtgggggctgcccaggggcagaagtgtgcatggcagtgccctgggagcaggccagcctgggagggcagcggccctgtctgggcacacagcgcccggccacaggtaggagcccagccacccccacctggggtctgggcaagaccagcagggactggggctaggaggcgatgtctggcaagcccgggtggctgctggcgcatgggggaaaaagtgtgggccacacagaggaggtggctcgtgtggccctctcttatcaaacattccgtgtacccagagggcagagcgcactggtaccaccaagacggcagctctcgttgcccccaagagcctcccactggggaggtctggctggaaacccagggaggggaccagaactctatggaggcaggaggcttagtgaggggtgctcagggaggcagccagaggcagaggagctcagggaggggatgacaggtcaggtccgtggaggggacagaggctagggagggacttgggccctggtgagcagctcgggctgggaagcaacttctctcaaggcagggtggggacaggcctgggcaacgatggtaagcgggtggctggtgactcagggatgagagggccttgcggccccatgacccctgcccgggacccagggagtgggcagcagctgtgagcagggaggagccctaggccctcagcggcctggggccaggcacagcatgaggctgattctagagccagggcctcggggacctagtgacaggaactgtggaccctgggagatgggggagcagcagtgaccactcgccatctcccacctggtccccctttttttgagatggaatcttgctctgttgcccaggctggagtgcagtggcatcatcttggctcactgcaacccccacctcccgggttcaagcaattctcctgtctcagcctccagaatagctgggactacaggcgcccgccaccacgcctgactaatttttgtattttttttttttttagtagaaacggggtttcccctcattggccaggctggtctcgaactcctgacctcaggtgatccgtccacctcagcctctcaaagtgctgggattacaggcgtgagccaccgcgcctggcctggtcccccttttaaagaaaggcagcaggggggcctgtaatcccagctactcaggaggctgaggcaaggagaattgtttgaacctgggaggctgcagtgagccgagatcgtgccactgtaccccagcctgggctacagagcgagactccatctcaaaaaaaaaaaaagaaagaaagagagaaagagagagagagagatgaaagaaagagagagagggaaagaaaggaaggaaggaaggaaggaaggaaggaaggaaggcaggcaggcaggcaggcaggcaggcaggcaggaaggcaggcaggcaggcaggcaggcaggtggacagcaagaagacaccgttttgccatgaggttagacacgcggacaggcacagagcagacgcacgtgcaccatgctatcatggcaggacaggttcacatggcctcagacggagcgacagacacccagagggtaggcagaggctgcgtgcagaccccatgctgagactccagggttcagcgactgcaacacacaggctctgcaccctcctgcaaagcaaagccactgacgacacacagggccgggcgcggtgactcactcctgtcatcccagcactttaggaggccaaagcaggaggatcgcttgagctcaggagtttgagaccagcccgggcaacatggcaaaaccccatttttgctaaaaaatacaaaaattagctgagtgtggtggtgcatgcctgtaatcccagctactcgggaagccgaggcatgagaatcgtttgaacccaggaggtggaggttgcagtgagccgagatggtgccaccgcattccaacctgggtgacagcaaggcacggtggctaacttcctcccctcgccctgcacccctttcccaccctggcattctctctgggcacctcctttctgtaccagcagatcagggaaagggagccaccaggggatgggaagggggacttcctgagctaagcctggtgctgtgcaggggaggggactgcttcctggggttcctgttcacacccacagtcccctgctgtctcccacgcaagctcattataatcccagcactttaggaggctgaggcaggaggatggcttgaggccagaagtttgagaccagcctgggcaacatagcacgattctatctctgggggatggggttgggggtgaagacacaaacacacacacacacacacacacacacacacacacacacacttgaccctgcagtgggttctgggtttggggtgaccttatctaactgactcctggtctccttggaggtggctcctgggaggcagacttgagcagctctcctggcaggggggtgggagggaaaaaactgggagctggggcccacccaggtctgtggggagttgtcacggtggggtcggaactgcccagactaagaggcctcaggagctggcacggatcacaggctggtcagggtcagagaaaggtgcccggaagggagcgggtgggcgggcggcgcccaggcccgggatgtggtcaggccgtggccagctgcttcccaaaaccagaatagcccaggacttacacaacccgcaccccaaacaatccgctactgcctcccccagctctcgcaggcttcccagaccccttgccccaccccactcagagcccacccttaggcccctctcgcccccaccccactcagagcccacccttagggcccctctcgcccccacccctttcggagcccagccttaggcccctctcgcccccaccccactcggagcccacccttaggccccctctcatccccaccctacttggagcccacccttagcccctcttgcccccaccccacttggagcccagtcttaggtcccccttcaccccccccccacttggagcccacccgtatccccctcttgcccacaccccactcagaacccacccttaggcccctctcgcccccaccccactcagagcccacccttagcccctcttgcccacaccccacttggagcccagccttaggcacccccctcacccccatcccactcagagcccacccatatctccctcttgcccacacaccactcagagcccacccttaggcccctctcgcccccaccccactcggagcccagccttaggcccctctcgccccattacactcttcaccccactcctaggctccacctgttgaccttccaacaaaccaagcttgtcccggacccagtgacctcacgttccccctcatctccaactcaccctcttgaacttctgccaccctggggcccctcccatgccaggcctcctcccctagcaccgtaccccttttccaccctggcactcgctctgggcaccccctttctgcacctccagcagatcagggaaggggagccaccagggggtggggagggggacttcctgagctaagcctggggctggggaggggagggggctccttcctggggtccctgttcacatccacagtcccctgctctctcccacgcaagctcattccacccaagacggttgctgcttcccctaatcgagacagaaaccctcccagtttcacaatcttctgtggaagggacccccaccctctgcccgccagtccccatgtccccacccgccaaggctggggacaggccactctcctctcagctgtttcccagccatagccctggccctaactgctgttgccctgtgtgtctgaattacagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggtgagctcaccccaccccgtccagccctgtgtagctcacttgagagctgttcctgccctgttcatgtccaggaggtatcagacactccctgagtgcccagtcttctgcttggagattggagggaaagaaagaccccatctctgccctctgggagggaccccgtgaagcagaactggacaaaacggttgtgggagaaagctaagggcatggagaccatggaatgggctccaccaagggccagttgcttgtggaaggcttcctggagaaggggagtttctgggccttagagaataggtcagctttggacagaagggactggctgacctcaagccagatgggagaacagcaggaacaaaagcttcgaggcagaaagctgagcgtggatgtttaggaaacagtgaatctaccattttggttcatcaattccccatatgcaattcttttttttttttttctttgagacagagtctcactctgtcgcctaggctcgagtgcagtggcatgatctcggctcactgtaacctccgcctcccgtgttcaagcgatttccggcttatttttgtatttttagtagagacagggtttcaccatgttgcccaggctggtctcaaactcctgacctcagtagtccgcccgcctcggcctcccaaggtggtgggattacaggtgtgagccaccacacctggcccccaggtgcagttcagaagttgcttcctgctgcttggttataggccttttccctccatcatcagacttttcaggtgctaattctaatcggttgctgaggggtgtctctcctccatcagacagtgagctccttactgaaagggttatgttttgctcaccactgcattctctatacctaatacagctcaggcacataacagcactcagtgaatggttgtccaatgagtgagtcagtgactgaatgaatgagcaagtgtgcagatgaatgggcgtgctggtgaatggatagatgcatcttagactgcggtggaggcacacgtgggttggtggatatatgggtaggtgagtggttgggtggatgggtgggctggtggatggatacatagatgaatagtttaatggattagtgaatgggttcattggtggttaagtggctggaaatgtatagatggtggatggatggaaggattatggatgggttggtggatgaatgatggaagtatagatggttggatggattagttaatatgttaatatctggtaggctggtggagagagtggtagatgagctggttcatagattgataaataggtaggtgaatggatggaagacggatggatggagagattggtggatgaagtagtagatggacaattgatgaatggatgggtgggttggttagtggatggattggtgaagttagtggatggatgatgaaggaatggtaggttggttggtggatggatcagtggacaagttagtggatggatggatgatgaatggatgggtgagttggttggtggatggattggtggacaagttagtggatggatggatgaataagttatttgggagacaaatggacgggcaggaagattgatgctccgctgttcctgctttttgcataaatgtactcgactccctcctctcattgtaatttccctgctgcatgtatatgttcgcacacctgtgtgtgttggaaggcagggctcatttctcctatctctccccagggccccagtctagagtttggccagggcaaggcagggcaggggaggcccagatggctgccaggggcagctctctcccctgctctctgagtgtctgtctgtccccccaaccaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcaggtgagggccagcaacggggagaggaaagcctggggagggaggccagggggtggggaggacagggggttttgggagcagaagatgaaggtgaaggggcaggtccagttcaagggacagagtggcaaggaatgagggatgtggggcctgtacctaaaaggggtgtggactcttgggaggggcaggatggggtcttgatagatgccgggggtgggccagccccagtagcctcacggttctgcccgtgcacagctaagccggagcaagccctggagccaccacttgtatccagcaccctcctccggtccccagggtccccggtaagatctcgggcactgggcggtggggctgtgcccacggccttcaacccgactctccgcccttctccaccccagcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggtaaagagaggggttgcagggattgggggaatctaggggatgaggggaagaggaggaagaagaaggggactgggtctggggccagatgagaggaagggggcaatggggaccctttcccctgggtcggtctttctgtgggtctgtctgtccctaccacaggcagtgaacggcggctgtcaggggacctcagctccatgccaggccctgggactctgagcgtccgtgtctctcccccgcaacccatcctcagccggggccgtccagacagcaacaaggtgggcaccatcccccaccccaagatcggtttgctacccccaggccctcctgggctccttcctaggctcacccaactctctgctccccgagaccgagaaccggcgtatcacacacatctccgcggagcagaagcggcgcttcaacatcaagctggggtttgacacccttcatgggctcgtgagcacactcagtgcccagcccagcctcaaggtgagccccgggcccaggctggcacccccaaaaccacagggcccctttctggaccccccccacccaaaactcaattgcaggggagtgctccagacactcttgcctcctcccatcccagtgcctcagtgggtcccagggtacagcctctagcacctgcctgagcccccctggtggcgtctgcaggtgagcaaagctaccacgctgcagaagacagctgagtacatccttatgctacagcaggagcgtgcgggcttgcaggaggaggcccagcagctgcgggatgagattgaggagctcaatgccgccattaagtaggcagggtccaggcgggtgctgggaccagggggccatgccgccttacccccagcccctgacccctgccctgcccccagcctgtgccagcagcagctgcccgccacaggggtacccatcacacaccagcgttttgaccagatgcgagacatgtttgatgactacgtccgaacccgtacgctgcacaactggaagttctgggtggtatcctccctcaagcccatggctggggggctgcaggggctgtggcagggtgagaccaggctgggcctgggatggaggcctagctctccaggccgggcaggctcagagctgctcccaggtgacaggctctggctctgccccctgcttccatgccctggccatcttggccagcaaaattgcccagggatggggggctgggagcagagagggaaactgagggaggagtccttgggtaaggggagccttggccctgacctcaggaagccccgagggcagtttgggagaatctgaactctggagtgaggactacagaagacttggaatccactccctgatctggctcaggggtcaagttcttgactgagcccaggcagggagctcagggatgggggatggttcagcacacagtaagggccgtggaggggcaaggaggaggggactgagaggcagagagctgggaaggtcttggcaggacagaagtagggactgcaagccaggtgcggtggctcacgcctgtcatcccagcactttgggaggccgaggcaggtggatcacttgaggccaggagttcaagaccagactggccaacatggcaaaaccccatctctactaaaaatacaaaaattagccaagcgtggtggcatgtgcctgtaatcccaactactcgggaggctgaggcaggagaacagcttgaacctgggaggtggaggttgcagtgagccaagattgtgccactgcattccagcctgggcagcagagtgagactctgtcaaaaaaaaaaaaaaaaaaagtccgagcacagtggctcacgcctgtaattccagcactttaggaggctgaggtgggtggatcacgaggtcaggagttcgagaccagcctgaccaacatagcgaaaccccgtctctactaaaaatacaaaaattagccaggtgtggtggcgcacacctgtaatcccagctactcaggaggctgaggcaggagaattgcttgaacctgggaggcagaagttgcagtgagctgagatcgtgccactgcacttcagcctgggtgacagagcgagactccgtctcaaaaaaaaaaaaagaagaagtaggggctgcaacatgagtcattggggttcaacgttagacagtgtccaggccctttcttcccctggtttgaactgggcatcagggctccacgtggtgtggggggaccagctggaccacaggcagctccttgacctgggcccagttcagcatcctcatccggcctctgtttgagtccttcaacgggatggtgtccacggcaagtgtgcacaccctccgccagacctcactggcctggctggaccagtactgctctctgcccgctctccggccaagtacgtgagccatctccctgcctgctagagggacccagcctcgggggtggagggagcattccaccctgcttagatcccacatccagcttgccatcaacacagcctagggttgtggccaccccgcatcccccagatggtcctctggccaccagacgcctgtagacagaggttctaggagggggtgacagtcttgccaactaatacctccccctatccctttggttccacatccaagggtagggccctgaccccaggaagtgtctgtctctctctctctgtctcccacccagctgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggtttgttggtgtgctggtcagtgtctcacactgggatggagagagagcctgttggcagtgggaagtggctttgcagagggcctgggcatttactctgagctggaagcagctggagggctttgagtggaggtgggacaagatcagatgtagtggtggcgagcgcctgtagtcccagctactcaggaggctgaggcaggaggatcgcttgagcccaggagttcgaggctgcagtgagctatgatagcaccactgcactgtagagtgggcaacagagtgagaccctgtctcaaataacaacaacaaaaaaggctgggcacagtggctcacaccagtaatcccagcactttgggaggccaaggcgggaggatcatagacctcatctcagtaataaaaaagagaaaaa

SEQ ID NO: 11 - skipped

SEQ ID NO: 12 - DNA, 3633 bp, M. Musculus
aatagtggccatggcgcgcgcgctggcggatctatccgtgaacttgcaggtcccccgggtcgtccctagcccggactcggactcggatacggacttggaggatccgagtccccggcgcagcgcgggtggcctgcatcgatcacaggtcatccacagcggacacttcatggtgtcttcgccgcacagcgactcgctgacccggcgacgcgaccaggaggggcccgtggggctcgccgacttcgggccgcgcagcatcgatccgacactcacccacctcttcgagtgcttgagcctggcttacagtggcaagctggtctctcccaagtggaagaacttcaaaggcctcaagttgctatgccgggacaagatccggctgaacaacgccatctggagagcctggtacattcagtatgtgcaacggaggaagagcccagtgtgtggtttcgtgacccctctgcaggggtctgaagcagatgagcaccggaaacctgaggctgtcatcctggagggtaattactggaagcggcgcatcgaggtggtgatgcgcgaataccacaagtggagaatctactacaaaaagcggctccgtaagtccagcagggaaggggatttcctggctcccaagcaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatgcttatgttggcaatgctgacatgatccagccagacctgacgccgctgcagcccagcctagatgacttcatggagatatcagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcagccaagcctgagcaagcactggagcctccgactatgcccggtaccctcctccggcctccagagtccccgcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggcagtgagcggcgactatcaggggatctcaactccatacaaccctcgggggcactgagtgtccacctgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtgcccatcacacatcagcgctttgaccagatgcgggacatgtttgatgactatgtccggacgcgcacgctgcacaactggaagttctgggtgttcagcatcctcatccgacctttatttgaatccttcaatgggatggtgtctaccgccagcttgcacagcctccgccagacctcactggcctggctggaacagtactgttccctacctgctctccggccaactgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtctgcaagggttggtgttggtgtgactggcaatgtgtctcagtatgggactcagagctgccagcagtgaaaggtgacatagaagaccgatcctgtgggttataaacaacctttgagtacttttgatttgttattttgtttttgttttttcgagacagggttttttttgtgtagccctggctgtcctggagcttactggataggccaggctggcctccaactcagagatccacccacccctgcctctggaatactggaattaaaggcatatgtcaccaccacccggccctttgagtacttttcaacttgcttgtgtgcagttgtgctcacctgtgcttgtgtgtggaggccgtcggctgacctgggtacctccttcgtgctctctgccttaattttaaaatgcttccgaatatattctagaactgggcatggtgatgcacacctttaatcccagtacttgggggaattc

SEQ ID NO: 13 - DNA, 3215 bp, M. Musculus
aatagtggccatggcgcgcgcgctggcggatctatccgtgaacttgcaggtcccccgggtcgtccctagcccggactcggactcggatacggacttggaggatccgagtccccggcgcagcgcgggtggcctgcatcgatcacaggtcatccacagcggacacttcatggtgtcttcgccgcacagcgactcgctgacccggcgacgcgaccaggaggggcccgtggggctcgccgacttcgggccgcgcagcatcgatccgacactcacccacctcttcgagtgcttgagcctggcttacagtggcaagctggtctctcccaagtggaagaacttcaaaggcctcaagttgctatgccgggacaagatccggctgaacaacgccatctggagagcctggtacattcagtatgtgcaacggaggaagagcccagtgtgtggtttcgtgacccctctgcaggggtctgaagcagatgagcaccggaaacctgaggctgtcatcctggagggtaattactggaagcggcgcatcgaggtggtgatgcgcgaataccacaagtggagaatctactacaaaaagcggctccgtaagtccagcagggaaggggatttcctggctcccaagcaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatgcttatgttggcaatgctgacatgatccagccagacctgacgccgctgcagcccagcctagatgacttcatggagatatcagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcagtggtcctcatagtcctgcctgtacccagccaagcctgagcaagcactggagcctccgactatgcccggtaccctcctccggcctccagagtccccgcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggcagtgagcggcgactatcaggggatctcaactccatacaaccctcgggggcactgagtgtccacctgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtgcccatcacacatcagcgctttgaccagatgcgggacatgtttgatgactatgtccggacgcgcacgctgcacaactggaagttctgggtgttcagcatcctcatccgacctttatttgaatccttcaatgggatggtgtctaccgccagcttgcacagcctccgccagacctcactggcctggctggaacagtactgttccctacctgctctccggccaactgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtcaaaaaaaaaaaaaaaaaaaa

SEQ ID NO: 14 - skipped

SEQ ID NO: 15 - DNA, 3227 bp, M. Musculus
aatagtggccatggcgcgcgcgctggcggatctatccgtgaacttgcaggtcccccgggtcgtccctagcccggactcggactcggatacggacttggaggatccgagtccccggcgcagcgcgggtggcctgcatcgatcacaggtcatccacagcggacacttcatggtgtcttcgccgcacagcgactcgctgacccggcgacgcgaccaggaggggcccgtggggctcgccgacttcgggccgcgcagcatcgatccgacactcacccacctcttcgagtgcttgagcctggcttacagtggcaagctggtctctcccaagtggaagaacttcaaaggcctcaagttgctatgccgggacaagatccggctgaacaacgccatctggagagcctggtacattcagtatgtgcaacggaggaagagcccagtgtgtggtttcgtgacccctctgcaggggtctgaagcagatgagcaccggaaacctgaggctgtcatcctggagggtaattactggaagcggcgcatcgaggtggtgatgcgcgaataccacaagtggagaatctactacaaaaagcggctccgtaagtccagcagggaaggggatttcctggctcccaagcaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatgcttatgttggcaatgctgacatgatccagccagacctgacgccgctgcagcccagcctagatgacttcatggagatatcagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcagccaagcctgagcaagcactggagcctccgactatgcccggtaccctcctccggcctccagagtccccgcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggcagtgagcggcgactatcaggggatctcaactccatacaaccctcgggggcactgagtgtccacctgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaagggcttgccaacacagaggcccacattggtggcgctcgcaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtgcccatcacacatcagcgctttgaccagatgcgggacatgtttgatgactatgtccggacgcgcacgctgcacaactggaagttctgggtgttcagcatcctcatccgacctttatttgaatccttcaatgggatggtgtctaccgccagcttgcacagcctccgccagacctcactggcctggctggaacagtactgttccctacctgctctccggccaactgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtcaaaaaaaaaaaaaaaaaaaa

SEQ ID NO: 16 - DNA, 3234 bp, M. Musculus
aatagtggccatggcgcgcgcgctggcggatctatccgtgaacttgcaggtcccccgggtcgtccctagcccggactcggactcggatacggacttggaggatccgagtccccggcgcagcgcgggtggcctgcatcgatcacaggtcatccacagcggacacttcatggtgtcttcgccgcacagcgactcgctgacccggcgacgcgaccaggaggggcccgtggggctcgccgacttcgggccgcgcagcatcgatccgacactcacccacctcttcgagtgcttgagcctggcttacagtggcaagctggtctctcccaagtggaagaacttcaaaggcctcaagttgctatgccgggacaagatccggctgaacaacgccatctggagagcctggtacattcagtatgtgcaacggaggaagagcccagtgtgtggtttcgtgacccctctgcaggggtctgaagcagatgagcaccggaaacctgaggctgtcatcctggagggtaattactggaagcggcgcatcgaggtggtgatgcgcgaataccacaagtggagaatctactacaaaaagcggctccgtaagtccagcagggaaggggatttcctggctcccaagcaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatgcttatgttggcaatgctgacatgatccagccagacctgacgccgctgcagcccagcctagatgacttcatggagatatcagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcagccaagcctgagcaagcactggagcctccgactatgcccggtaccctcctccggcctccagagtccccgcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggcagtgagcggcgactatcaggggatctcaactccatacaaccctcgggggcactgagtgtccacctgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaagctcccagggcttgccaacacagaggcccacattggtggcgctcgcaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtgcccatcacacatcagcgctttgaccagatgcgggacatgtttgatgactatgtccggacgcgcacgctgcacaactggaagttctgggtgttcagcatcctcatccgacctttatttgaatccttcaatgggatggtgtctaccgccagcttgcacagcctccgccagacctcactggcctggctggaacagtactgttccctacctgctctccggccaactgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtcaaaaaaaaaaaaaaaaaaaa

SEQ ID NO: 17 - DNA, 543 bp, M. Musculus
tgtctccccctcaaactgtcctaagccggggtcgtgtagacaacaacaagatggagaaccgacgtatcacacacatctccgcggagcagaagaggcggttcaatattaagctaggattcgacaccctgcacggacttgtcagcacgctcagtgcccagcccagcctcaaggtgagcccccagccagcaatgcacactcccttagagtcgtggaacccctccttggaccaacacccaacaccagctcccagggcttgccaacacagaggcccacattggtggcgctcgcaggtgagcaaagcaaccacgcttcagaagacagcggagtacatcctgatgctgcagcaggaacgggcagctatgcaggaggaggcgcagcagctgcgggatgaaatagaggagctcaacgctgccatcaacttgtgccagcagcagctaccggccaccggggtggccatcacacatcagcgctttgaccagatgcgggacatgtttgatgactatgtccggacgcgcacgctgcacaactggaagttctgggtgt

SEQ ID NO: 18 - DNA, 32001 bp, M. Musculus
agccagactttctgaaacctgaaaagtagagactaaaaagtgctctcaacagaaagaaaggcgaagcaccgccaaagctccaggatcagtggtttcacaccacgctcactggttatgctaatttaaacatcacctgccaactaagggccacgcccaccacaaggacctatcaggagaacaggggcgtggccatggtaattaggcgcagctggctacgttaagcgctctccagagcgcccgagagttggcgacaatagtggccatggcgcgcgcgctggcggatctatccgtgaacttgcaggtcccccgggtcgtccctagcccggactcggactcggatacggacttggaggatccgagtccccggcgcagcgcgggtggcctgcatcgatcacaggtcatccacagcggacacttcatggtgtcttcgccgcacagcgactcgctgacccggcgacgcgaccaggaggggcccgtggggctcgccgacttcgggccgcgcagcatcgatccgacactcacccacctcttcgagtgcttgagcctggcttacaggtgagggcggaccggggagcctgagtcccagctttccgtggtgggaccgcgcactggggctcctgtcccaaactgctacgggggacgctggacacaacttaggcattatcttcctcgcctggtcctgcgcaacccacggaccctcatgaatcatttgtgtcccatcacttgcttgtggcgccactgtcctgtcctccacctttgtctccacctttgcagggccttctacggttgggcccccgctctttgattcttctgcttttgcacttaaatgccaggtgtctttcacactcctcagactataacccatcccgtccactgcctccctccactcgtggtatgcctggactgtttcgttcatcctcagcagtccctgcaaagtcccactcttccctgcacgctgcagttatcccgtggtcgccctcataggcagtgtccacttacatggaaacagtttcttgctcacttgcccagacaccatttattttggcttgatttagtttggtgagccagagtctcactctaaagttcaggctggcattgagctaggtagcccaggctggactcccatctcggcctcccaaatacggggattacaggcatgagccaccgtgtctgatttagaacgctacgtcaaggttggcgggaagacggtgcccagcacctgcccagagccagcagagggtgctgcagtccaccctaggtcgggtagggaaaacctggtctccccgaacccggtaagaggctgcaaggcagccaggaaccctgtaactacggctcagacctccggggccgcacccagcttcagccccacctatgtgtccccgaggagggcgcgaactggggtcttggcccagtgccctggagcaaagttcccgggagcagtgggaagaggataaattatcaaccgaaaacagtaggagagaaagggggatcggccggccttgatctttcttctctgtaaatatcacgcttcttcagcgagcctcctccccttcgtagaaccgctggctgtagaccggcacctgcagccccacaggggacctggagctggggtgggaattgcggaagagcgtggcccacctcgtggcggcagagccacgcgaccccattggctggaggcagagtaaccttggcccacaagttctggcctcaagcccaggtcatgttgcaacctgatagttcagggcgctgagaccaatgtggaaagttcaaattccctgtcccgttgcccctcccccgcccttggacatcatgagactgcctgggaaggggccatctaaggtctcagaacaccagaagcagctcacactggggcagtgctttaattatcaaagacttccgattaaaggagagggagaatatccctcactctctttctcttattgagtcccacactgtagactaagctagcctggaactcactgtaaaaccgaggctggcctggaattctgctctggccttccaagtgctggaactacaggcacgagccacaagaagccttgggtccccatgttcccccaaagggtctcccgtttcccgaccactcccaactcaggcttcctgcttcggccatacattccacttgggcaggtttagagggctttccagctcagaccttgggcagacagaagggaagcggttttagagaaagagcacgtggagggtctgagggcactccctaaactgcagctccctagggtggagagtgccgtgtcccccacccgccaaagagccagactattaaattctcttgagtgggattctgaggatacccaagttagccaagtacacaggagtttttggtgaagaaaaaaaaaaaaaaacataaaacaaaacgtgggccaggagaggctctgccagggagggtgcttggtcccggactggtgacctgagtctgattcccagtacccacatcaaagtggctgcagggaattgactccatagttgtctgaactctgacctccatgtgccacccacgagctcccctcccagtgagacataaaaataaaaacaaaagcaaatgccccgtgctcacgctcatgaaccctgtggggttgggcatgcctagaatcctagaactcaggctgaggatggagggccaggaggtcaaggttgccctccactgcggaggaacttaaggtctgaggccagctggagatgataggagatcctaccatctcaaacaaacaaacaaacaaacaaacaaacaaatctgcggactgtaagatgtgactaggggttctctgaagccaagggtattgcagagggtgaggctctccagtgaacagctagaaatgtggggttggggcagccaggcagggggtctggtggtgggtggaactggtcagttaggcagggagaaggacagcctgcagacagcccacagaggcctgggcacctcacccctgccaagaagcgctcctggcacactctgttccctgcttctgtgggtggaagccccagggaggatagctgtaaatggtccaagaaggcacttgtctagggagtggtggcataggctggtcatcccagcagtctgtaggctgaggcaggaggatgaggctccagaccagcctggggctacagagcaaggccccgcattaaaacaaacaagctagctggcaaaaattaaaaaaaaaaaaaaaaaagagggacttggtgtaaagttatttgtcttacaagcttaaggacctgagttcaagccccagaactgacaagaaaaaaaaaaaaagagtctggtttgctggtgtgtgcttgtgatcccagtagtcaagacagagatggctagccagctgcctgtagcctgccagctaagcagcctagcttacttggttggtgagttccaggccaatgagagagactgtctcaaaaacggaggcagattcatttgaggaaccacgtttgagtttgtcctctggtctcacagagtgtatgcaccccacatcctccctctctcccacttctgaggttttctggttcaccccattgcacacagcttttgaaccttggccctagactttttgcaccaaaccggactcttctctcacgtgcattcaaccaaatcctttcctcgggacttggatattgtcactgactcatccccctcatcctaggctaccaaccctctgcgggagttggaggagggacagcagaaggctggggtaggaccctgggggaacagaacctaatggttgctgggatacagacagaggtcacaaacttaggagtgtcccccatttactctatatgacaacaggagctggcccaggaaggattagtgtccccaacccaatcatgttcacttcttggacagatggtatttatctagctatgggaggtaggggaccagccatcaggctagggtggcctatttgggtaatgaagcccattgtctcctgaggtggcagcccctgccaccccacgtcccacaccctggcagccaccattagctcaaagcaaatactggagcttggagacgagaaaggagaggagagccaatgacccatgcactcaaggtagtgcaggaagaggaggagggctcagatgctcacccctcatcccttaccctcatccctcacccaggggagctacccagagagcaggccttcgtgtgtgttgtgtgtgtgtgtgtgtgtgtgtgtgtgtgtgtgtctatacagtctatactgtctatacagtgttgaggatggaatccagtcgtgtccagttcctgggaaagctgccactgagccacgcccccagcccctcactggggattctagaaagtgctctgttggcagagccacactcccagccccagcctgactttctggatggctaaacctttgaagctttgagtgaggagtttgaagatgggccagaatgactcggaggcaagattggtttgtagaccatgaggaaggaaagtcaggactaaagataggacaggaaggtgtccaagaccttgattgtcttcatcttggcctcttgggccagcttccaggtactgataacttagcctgttctcagaggaaccttgagagtggaagccattgcttgtgcataccacctgcacaccatatgctcgctcagtggacccctagataaggtcgaggttaagattctatttctagccaggaggcggtggcacctgcctttaatcccaacactcaggaggcagaggtggacagatctctgagttcaaggctagcctggtctacagagttgatttcaaggccagcctggtctatataatgagttccaaaacagctagggctacccagcaagatgctctaagtcttaccgtatccctttggaggtatctctttccctctgtgaccctcaaagagatagattatgtcattggagctagagacatggtttggattgattaagagcactggctgctcttgcagaggacccaggtttgcttcccagcacccacatggcagctcacagtcatcagtaactccgttccagggggtccgacgccctcttttgttgctgtagccaccaggcattcatgtggtacagatggactctcatacacataaaataaaaataaaaccttttatttaaaaaaagtgtaagagccagcctggtctacagagtgagttccaggacagccaggggtacaagagaaaccctgtctcaaacaaacaaacaaagtttagaaggtgtaagagttgggcatgagaaagtgggcacgcctgtaatcccagcattctggaggcagaggcaggaggagcggaagctcaaggccagccttggctacatattgagcccgagatgacacactgaagactcattctgctatctgaagatgagataaagaccctgtgttgggatagagaggtggctcagtggttaagaacactgactgctcttccagagtcctgagttcaattgctcacacccacatggtggctcacaaccatctgtaatggggtctgatgccctcttctggcatgtaagtgtatatgcagatagagcactcatacacattaaataaataaatcttaaaaaaaagaagaccccctcccccaaagacaaaagaaagctgtgagagccctgcatggtgatatatacctcctataatcatagtattgaagatgcaatgctgaggcagaaggatctctggggcatttaaagtctttttattagcatatgttagttacacaagagagcatttcattatggcatttttatgcacgtagacaagaaatttagatgacactctcttgtcttctctcatatcagctcatctccttcctcttcaaaattctcttttactttcatgtcttacttggtgctagggatggggggtagtctttgtgtattcctggctgtcctagatctcactctgtagaccaggctggcttcaaactcaagagatctgcctgtttctgcctcccaagtgctgggattaaaagtgtgtgccaccaccacctgtcatcatatcttacatttaaaaataacatttatttagtgtgtgtgtgcatgtgcagggttgcagtgcatgccctggtgtggtggtcagagaacaatttgtgagctctatagccttcaagcacatgggtcatagggatcaaactcaggtcatgaggcttggcagcaagcacccttacctgctaagccatctagctggccctcctgctttcttttgtacttttgatccagtgagtgtagggttgctacaggtttgtggctgaggggttatttcagagcacgaccatcctaagtgtctacagcacggtagaaaccatctccccctcacccccctgtacccactcgccacttataggacagagtggagcctctagagtgtctccgaccacgacgagacaatgtgatgtctaattccatgccatcctgggctacctatagaaactctcgggcttgacagatggcttagcagaaggagctagccaatagctagctgagctcaattcctagcacccacatggcggctcacaaatgtctgtaactccagttccaggggatctgacacacagacagacaggcagagactccagtgcacacaaaaataaatctgaaaaaatctgaaaggggagaaactctttcttcttctacacacacacacacacacacacacacacacacacacacaccccatactggatgtgaaaactcttgcctttaaggccaatggacggtgttaatctggagacaattgataacaagtacctgccttcggatgggaggagatggcccatggacaaagggtgggctttggatgggctgttaataaggtttgacttcaggatggggatttcgggctgttaaataatagatgggatttgaggactgcagaggaattgtagccatcacggcatgctactgcacaaatcctcccgtcgtcttttctggccccaccctgatattctttctgctccctctgtctaacagtggcaagctggtctctcccaagtggaagaacttcaaaggcctcaagttgctatgccgggacaagatccggctgaacaacgccatctggagagcctggtacattcagtgtgagtgggtgccatgggtcccttgtgtttgggagggctccgtctagtcagagggctgcacccatggagccaagagccagcgcccatgcccatagggtgcacagtgccagccttgtgggggagagggaactgcgcaaggacccttgaacagcttccagccttcagagggtcttgaggagtctgtcccaaaggtcacttcccactgtgagacatgatgcggcaaagggcaccttgaaagctctgaaggaggccgcgccgtgggtgccgggtcaagatccggtcagcagccctttcattgagtaatctacttggaatgctctggtaaaaagaagtcagttcgggccagaaatgtggctcaggggtagaacaccagcccagagttccccttactcagtaggagagcacccgcctagagtcccgggtttgggggggggggggtcgtccagtggaaaagtgtctgcctagaatccaccaaggagtgacggctgccggatgtggatcagcagtaacacatttactaagcatattcaagccccttggttccaactccagtaccatacaaagggtatgggggcatttacataccatgccggtcccattccaatgctttgtaagtgaggaaaattctgcagggctcaccatcagcctttctctgtatagcccagagctcctccatagaccaggctggcctcgaacacagagatcctccctccctctgcctccggagcgctggaattaaaggcttgcaccaccactacccagcaccgtcagtctttcaatgaacaccccgtagggacctgctgggatttaggcagaagtccataaagccatgttgaaaaagacgctggactcagagttgctcagatgggtttcgaggctctgcatcttactagctgtgtgatttcaagcaagttgcctaatctcttcgaacttacttccctagctagccatgaagccaatattcctaacttctcatggctggtatgataaaaacagatataagtgggtatcttagttagggtttttattgctgtgaagagacaccatgactaaggcaacacttttttttttaaagatttatttatttattatatgtaaatacactgtagctgtcttcagacacaccagaagagagagtcagatcttgttacggatggttgtgagccaccatgtggttgctgggatttgaactctggaccttcggaagagcagtcgggtgctcttacccactgagccatctcaccagcccaacactttttttttattatttatttttattttatgtgcactggtgtttgtcccttatatatgtatgaggatgtaggatcccctgtaactggcattacagacagttttgagctgcccagtgggtactgggaattgcacctgagtcctctggaagagcagccaatgctcctagggcaactcttataaaggacagcatttcattggagctgacttacagttttagaggttcagtccattgtcatcatggtgagaaccatggcagcattcacgcaggtgacacacttcctctaacaaggccacgcctactccggcaaagccacacctcctaatattacccctccccatgggccgagcatgcaaacacatgactgtatggggggggggcatatctattcaaaccaccacagagaacctagcattgagcggaggttttgtgaatgatatgacaagaagaggttacagtcatgtatttggggacagcccctctgtggcaaggcagtaagatcaaaggttgtgttgagccggaaacacagctctggaggtaaacacatttacctttctctttaccggccaggctggatcccatccaagcaccgtggaactgttacactgtagcttctattgatccatgtgacctgcacttgaataaattatcttatttaagactcaaaagaggccaggggtgatgacgggagtctacaggcagaggcaggaggatctctgtgagtttgaggccagctttgtcaacgtagtgagtgagtgtcagaaccgccaggtctacaaggtgagatcctatggagatcatagaagcggcactgagagtgacaagggcagcttgcaaacacccgactgggcagcctcaaacgtggcaagcagaagcaggggagcatccttcatccatcctgctgtagcaagttccgggccagcctggactaggtgaagtcctatctgacaagaaggtgggggtgtgagaacacgtcctcagggagaggtttcaccctcccactttcagctgtccagctgtgtcacttgcccagcatcgtccagggaaggacccacagccagtgggggtgtggctcaaccagggagcacttgcctagacacccccctccccaggcacacaagaactacctgccgttttgcatggcttctctgggtctccaagaagttatctgacagactctaggcaaattaggaaaccggaatgttgttgaaatttttaaaacttttttttaaagatttatttattttatgtatgtgagtacaatgtagctgtacagatggttggtaagccttcatgtggttgttggaaattgaattttaggacctctgctcgctcagtccctgctccctccggcccaaagatttgtttatcattatacataagtacactgtagctgtcttcagacgcaccagaagagggcgtcagatctcatttcgggtggttgtgagccaccatgtggttgctgggatttgaactcaggacctttggaagagcagtcagtgttcttacctgctgagccatctcgccagccctgaaatttttaatcccaatccaagatttcgaccctgcctttccccatttaattcctggatgaaagacacacacaacctttatatttacaataagccttactcagcactagagctgagcagttatctaccctctatgctattaatgctttactgttctattgataaccctgagttattactttctgtgtttcatctgggctgctctgaactccaattggccagccctcaggccacgttttcatgactcacctaacccatggtggcttctctctctcccttcaccttctctcccctcatagttttcctctgaccccgggggggcccaacaatcacccagcttcttttttttaacctaaattgtgagcatcgagcatagaccctgatgcttccagagcttgtactgatggcgccatctcctccctctcttctttcctcctttcaaaacagggtctcatatagcccaggcaagatctgaattcagtaccaaggtgaccttgaacttctgatctttctctacctccttagtgataggcatacagcgaatcaccatacacgcagcctactgtgagcttgcttgctttctcctccccttcttttgagacagggttttactacgtatccttgactgtcctgaaacttgctatgtagaccaggctggccccaaactcacagaggtgggcctgcctctgcctcccaggtgctggtattaaaggcgggtgccacaatgttaggcttttccaagtggttgtttgtttgtttgtttgtttgtttgtttgttttttactgcttagttctcaggtatgtctgtgcatatgtgtatcacttagccggctccttggaggtggggaggctgggactgctcgttgtcattatgatcagagtggcaatatgttcttgtcttagagcaaagcctggtgtgatggtatagacgccagcttcctcaggctgaagaaaggtctggaattcaaagtcagcctgtgtaacctactgagactatatctcaaaataaaatctataatgtaagcaagagatgtagctcattgatagagtgtcagcctagaatcccccagggaggggttagaggtgtggcttagcatggtttagcagtatcacatttgcaaagcatgcaggaggtcccaggtaatattcagcacaattaataaaccagtcagtaaatatatttgaaaaattaggaagctagaaagccaggtgggcatgacttcccccacctttgagtttctggggactggagagatggctcagtggttaagagcacttgttgctcctgtagaggacctgggtttggttcctagcatccacgcagtggctcctaaccatctacaactccattttcaaaggacctgatgccctcttctgacctccacaggtaccaggcacacacacacacacacacacacacaaacacacacacggatggacggaaaacactcaaataaatgaacctttgaaatattttaaatatctaaataggtgattgtgggccccagcagaggtggtgtgcacttttactcccagcactcagaaagcagaggcaggcagatcttcgagttcgagaccagcctgatctacatagtgagttcgatgatagccagagctacacagagaaatcctgtttgaaaaacaaaaaacaaacaaacaaacaaacaaagtgattgagatgatggaagtctgtaatatctgtaggtactgtagaggctacgggagttcagttgggactacacagaaagacagtgtgtgtgtgtgtgtgtgtgtgtgtgtgtgtgtgtaggcaggtaagatggtttggtggagagaggcgctttccactaagactgacagcctgaatttgcttcactggatgatacgaaggtggatggagagaactagctctagaaagttctgacctccatatgtgcaccatggcacacatgtgctcctcttccctcccacacaaaatgaatgatggtcataaaaatgtagctcatcataatagtggtaaagctgtggatgtaactccagtggtacatcctgagcagtggtggtagctccagttcagtccctagttggagaaagcctggctcacccccccccccccagcagaaagtgtcatctcatatcacccagcagggggcactactgctccactgcagttccctaagttcgttgtgacctgcggtgtccttccctgtctgcgccactcctctcccacactcccaaagcctcttggtttctcctcagagctgccaaggcactttgcccttctccctggcaggagggactttccacttggctttatctatcccatgtttgttttccttatctctcctcctactctgagcagccccgatttatccctgatcctctcatcccagtgttggcccctgcaaaggtgcaaagggttagttagtgaatggcggccagtgggggaaggagcaccaacctggaatgcccccagggaggggctggggacaaggaggggtggcagagcaaatgcacagagggtgtggttggtgttacataaaacctgtcacagccgtctgtgcagaccacagggtccctgagtgggggaggagacaggcagggcccagtcgttcctggatatgtatactctttcctggtgatcatgtttgtatttgggaagtcaggagggagatgttctggaaccttccctgctctaggaccctactgatctacacacacaaaggtccatccctttgtcttcgcttcttcccttccacctccctttcttctttgttggatcttaggtagcccaaactaacttaagcttcgtgtcacattttgaccttcctgcctccatttcccaagtgctgaaagtgtaggcgtgtgctatctcattggcatgaaatttctagtcccccccatacctctttccctccctccctctttctccagctcctcttctcttttattcctccctccttttctctctttgtttgtgatactgaggccagaacccagagcctcgaatgtactagccatactctataccactgagctacgcccccccagcccctccctgggagattctaggcaggggctctaccacagagccacgcccccagcccctccctaggggattctaggcaggggctctaccactaagccacgcccccggcccctccctgggggattctaggcaggggctctaccactgaagtacatagcacccagcatggcctgtgatgaaggcctgttgagttaattgaccctgaagagaaataactgacagtggaagaaatcacagtaagaactggaggaaggtgtttttggtagaaggtgtttctgtcttctagagaagagtcttgtggcatggctcatgcctggagtagcattcaagaagctataaggcaggaggatcacaagttcgaggctgtctgggactatattaggacctgttttataaaaaaacaaacaaataagaaagagtgtaggctatggcttcagggtgggacacatgatggagatatgggaagctgggcagccaggtgaggaggaatttggaaaccaagcggggtctgagaggtgtgcttccgaccctggaggctgtggggggctatggaatgttttagagtggtggcagcagaaatggtgagcggctgcgtaagtttaggcttgagtcaagggaggtccattcaagtaatgattgagctaggggatactcgagaaaggatggagaggaggggacagtggatgaactgtcacgagccctgttgagttgacatggttggacgtggaacactgaaagctgggaaggaagttttgcaagtttttgggatatttgcaattcagggagcagcacagagcactcaggaggcagatcaaggcgggagctccagtggcttgtggaggaggtgcgtgctgtacagggtaggggtagagggatggagttgggccttctgcggagacactgagatcccagcgctgaagatggggcgggaggggtgggcacatgtgacacctgcagaaccggagcgtgagaaggacaggtccactccttagcgcaacaaaagaggaaaaccgtgggcattgggaaagagcccagagctaaagcaaagtgggaggtttgagatggctcagtggttaagagcactggctgctcttccaggagtcctgagttcaaatcccagcatccacatggcagttcataaccatctataatgggatctggtgttgtagatgtgtatgcagacaacacacattatttatttatttatttatttatttaatgaataaagttggaagcctgggctacatagtgggtccaggctagcctgaactataatagtaataatcggtgtcaaaataaaaaataattttcggtgctggtgagatggctcagtggttaagagcactcactgactgctcttaattcaattcccagcaaccacatggtggctcacaaccacctataaggggatctgatgccctcctctggcccatggatgtatatgcagcagaacactcatgcattaaataaataaaaagaaacgagtgtacacaaggcttagtgtgtatctcctcagtacactttctcagaaccatccactgaggatgggggtgtggctcagcagtggcgtgcctggactgtcccagtgagagacagggagagagagagagacagagagacagagagagagagagacagagagagagggggagattcagtagtagaacttgtagcgagagataggaggaggcagggagggaagagaagtggctcagtgggtagtatttctagcatgcaggaagctctgggttccttcaccatcgctgcataaaatgtacatgacactgctcttgggaggttgaggcagaaaaacaagtgactcaaggtccctaatgactttgagaccagcaaaggaccaatgtctgaacaaaagataagggtcaggcaggcatcgacttccagtctcgatgggtggaggatgagaggtacgggtgcctctgacctggccttcttccctcagatgtgcaacggaggaagagcccagtgtgtggtttcgtgacccctctgcaggggtctgaagcagatgagcaccggaaacctgaggtagctggagaatgggctgcccgcatccgcttatccacccagaggttccctcccgcggtggggattgggtgtgagatgggaattggggagattgaccatctggggtgtcacccttcgcactgatccctgacacctgcccttccccaggctgtcatcctggagggtaattactggaagcggcgcatcgaggtggtgatgcgcgaataccacaagtggagaatctactacaaaaagcgggtcagtgggactgacacctgcagaccttgggtcttaagagtcctggaggggatcgcacgacctcaccctaacccatacctttgagccaccccagttagtcccagcctagtagctgcacagggtttccgcagccctgcaccatggtggaaaggggcggggtctggggaaatagcaccaatggatgtgggtggatggtggatgagtcgggacagaggatccagtgtataactttgtgtcttcatagctccgtaagtccagcagggaaggggatttcctggctcccaagcaggtgagtgttccgtaagtctagcagggaacctaggaacatggaagctggagaatggaaaatgtctgcctggctgactaaaggcggggtctggacaaggtgactggggtatgagcagtctctaagttaggtgtggatcatcctgcaactttctcttttggagaagatccagttgtgaggtaggaggatggcagtgagttccaggctagcctgcattaaataatgactccaacgccagtgtgggcttcagagacctgtcttaagaaaagcaagcaggtagtgggaggcctagggggagagggtccagaggacagatgtctcagaagagctcaagcagaggacagacatctcagaagagctcagatagaaagccaggcatctgctgatttcaagtttacggtcagctcaggaagtttgaggcaagcttaggggccctgtcctcaagaactaaaacaattgccttggcttgatggtgcagataagcagtgctaccactaaggaggtagaggtagtaggatcatggctatcctctactacacagagaatccaggccagcctgggctaccatgaagccctacctcaaaaaattaaagccagcaagatgtgttggtgccctttttttgggggggggggtttcaagacagggtttttctgtgtagtcctggctgtcctggaactcactctgtagaccaggcggcctcaaactcagaggtctgcccgcctctgcctcctgagtgctgagattaaagatgtgcgtgcttgggctgccaccacccccactgccaccaccatcgcctagactggtgcatgtttttaatccgaaaacttggaaggcagaagcagccaggtctttgtgaactgaagccagcctggtccgtctggtctacataatgagtttctgaccagcaaggtctatatagtaaaaccctttataaaaaccaaaaccagaacaacaatgaaaaaacaaaaacaagaaacaaacagccagccagccaggcagtggtggcacatgcctttaatcctagcacttgggagatagagacaggtggatttctgagttcgaggccagcctggtctacagagtgagttccaggatagccaggactacacagagaaaccctgtctcaaaaaaaaacaaaacaaaacaaaaacaaaacaaaaaaaaaacaacaacaacaaaaacaaaaagaaagaaagaaagagagagagagagaacgaaagaacgaaagaacgaaaggaaggaagaaaggaaggaaggaaagaaggaaggaaggaaggaaggaaggaaggaaaaaaagaatcagctagattgtagtatagagaaaaggaaagcctattggcgcccccctctgctttatacagggagtacttagaggccgataggatgaccttagccttccagacagagaaccaggttcgccaggaaaaagggaggtggtgtcctttagagaggctttctagccggtatggggaaccacttcatccccaggcaaggggtaacatggaagaccagggcagtgacctctgtgtcctgccaggtggaaggtgggtggccgccaccagagcgatggtgcgaacagctcttctccagcgtggtgcctgtgctgcttgggggctccgaggaggagcctgggggccggcagcttctagacctggactgcttcttgtccgatatctccgacacactcttcaccatgacacagcccagtccttcgtccctgcagctgcccccagaagatggtgaggagtgactgggggtagcgaggggagaaggctgagacggcttcccagcctgcagcctccaacctcaccactgtccctccagcttatgttggcaatgctgacatgatccagccagacctgacgccgctgcagcccagcctagatgacttcatggagatatcaggtgagaggggtcctttgccactaggcagggcagactgaaaggaaagggtcaggttaagtaaaaactctaggagactgtaaggctttgcaccagtgggaagatgaatggtgctttaacttgaagtggtagggggaaaggatgtattagttggaactcaagaccctgtgcctgctaggcaggggctctaccattgagccatgcccccagcccctccctgggagattctaggcaggagctctaccactgagccacgcccccagtccctccctgggggattctaggcaggggctctaccactgagccacacccccagcccctcactgggggattctaggcaggggctctaccactgagccacgcccccagccccttcctgggggattctaggcaggggctctaccactgagccatgacttcagcccctccctgggggattctaggtgtgggctctaccacacacatcctttatgatctggcatgggagaatccttgggttcaatcctgagcacttcataagccactgtgactgtcaagagaattcgaggtgtcaaatctcttgtctatttttgagacaggctctcgcagtgcaactctggttgttctggaactcactgtgtagactcggctggcctcaagctcacagagggttgcccgcctttgtctcccaagtgctgggattaaaggcctgcaccactacatccagcttagaagcatcaagggttgtttgttgttgttgttgttgttttaaagcattttgcaaagtggcaggttttcccagactcctcttttctcaggctattgggtttctgatgtgtctgagtttttccagataccctgaccctgtcagtgcgagtctatgagaacaattctcccccagaccttcttttgagacagggtgtttttctttctgtttctatgacaaatgctctgaccaaaggaaaggaagggtttatctcaccttaaaggttacagtccgtcactgagggaagtcagggtgggaaccttggaagaatgaattcgaggactcagggctcctgttgctgtgatgaaactaaaacatcatgaccaacagcaacttccatatcatatagtctatcattggaggaattcagaacagaaacctggagcaggagctgatgcagagaccatggaggaggctgcttattggcttgcttccctaggcttgctcagcctgctttcttatagaacccagaaccactgccatggtgggtggggccctcccccatccaccaccaattatcacaatgcttcagaggctcacctgcagcctgatcttacggaggcatcccctcagctgaagctcccacctctcagatgattctagcttgtatcaagcggacataaaatgagccaacacacttagctacctccacatcagttaacaatcaagatgatccctagcagacaggacaactgaccagtttgatctaagcagtccttcagtcccattcccccccttcacgttaccgtaggaaattgtcaagttgacagttaaagctaactaggtaacaaggatatgagccagtcctggctggtcttgaacttgctgtgtggccaagctgtcctgaacgctgatcctcctgagtactggaactaaaggaacgtgtgtatcaccatgcctaaatcgggactagaggatagacgtgtgtttcatcattcctagagcgtgccctctcccttttcctctcccgttccctcccctactctccattcattctccccatcaccccagcacacacacacacttcttatttgagatagagtcactatgtagcccaggctggccttgaactcatgatccttctgtctcagtctcctgggtgtctggattccaagtgtgagtcataatacaacttgcttcctgttttctgatagggtcttccttccccacactgttgtcttgcctgctctctctctctctctctctttaaatttttatttatttatttatttttagtttttacatttgttgtttgtttgttgttgttgtttggggcaggatttctttgtgtagcctttgctgtcctggaactcacagagatccaactgcctctctgcctctgcctaagtgcactcgccaccacccagcactgtgtgtatttcttgctcgctgcttcttatggggatttatttgggggcctttaaagctccctaaatgttggggctcaggaggatatcagaatatcttacccaaacctgaaaagatagagactttgagagagaagcttcattcgagtgacacgccccctcccagcttcttccctacagggtcctgggcctgtcattcaagaggtgaggcctatgggaagggtactagggaatgaggaaccacccccacccccacctccccggccatagccctgggagggtggggtctgccaggggcagaagtgttgatggcagtgcccgggaagcagacttgtcccccaggagagccgctcactcttggcccacagacccctaccacaggtaggagctcagctgtgcccacccatgctctgggcaaaatgtggttggttgaagtttgcagtaacttgccaatcaatcctaggtggccgctgattgggggcaagcctggctctcccagaacaggtggttacagtgaccctctcaagctggccatgctacgggagaaccttgagccatgagggtttccactctcgtctttctggaaaccctgtggaaggcaccaaattccacagaggcaggaggttgggggagggcaggttcaggggcagagattgtggctgaccagtgtgaggaccaggtcagggaaggactggggtttgatgggagactgggagagggagggaatctctggaggctgctgggaacaggtgtggacagctcacagtacaggagcagtctgaggctctgatatgatgggctggtttgccagaggccaatctatgctctaatcccgggctccaaatgacttactgacaaggcagagagcaatggaactctggtgacccctttctggtccttccccatattcctttgtaaaatgtggggacacaggacagtgtccgatactgggagagatgtgggcatgccgggttcgttcagtcatatctgagggacagacaaccaaagaggcagacagcttcttgtcactgtatactggtactcagagcctgtcttctggccaggtagaaccacctaagacaaacacccacacgaatacacacacacacatacactcacatatattcacacatgccacgcataacacatatattcatacacaagcatcactgcacacaaacactttatgcacacgcacacacacatggtcacacacatatgcgttcacacatggacacacatgacaacatacatacatataccctacatatgctcacccacatgttcagaggcctacatgtatacatatatgtatatacacatgtaatcatatacattgtatacacatatatgcacattttaacatatgtacacacacatggatatacacatgcacatacatatatacacctgatgcactcccatatacactctcaacatatgtatatacataaatatacacaccacacaggcatacgcaccgtgatcctgtgaccttccctaactaacttggtgttgagaggtgatgtatgtcagaaaagtctgagcagctctcctggctggggattctggagggggtagctgcagcccaccaaaccctgcgagaagctaccatgttggggtctgctctgactggcccaggaagccttgggcacagaatacaggctggccagggtcagagaaaggtgcccggaagggagagggtaggcgggtgtcgccccagcccgggatgtggttaggccatgagcagcttgcttcccaaaaccagaatagcccagaacttacacaactcacacccctaaacaatctggctgcctcacccgcctctccataaacttttcagggtccctggctccctgccacttacaccccaactctgagcccatcctgtgtcccccaaatgtcagccaggactttattccatacagcttcagtttcctgatctctcagcaagtctaacctctccctgacctactgacctcatgaacctgttcacccttgattcatccctgacatattggggcaactcccctgtaccccacctttttccttcacagggcctctctgtgaccccttctttcaagaatgccaacagtgctagaagggggagcagacacagggaggggaagtgatctagcattgggactccatgggtgagcagcttcctggggctctcacatactcctagaccccaccatcttccccagggcaatcctatctatctctacctcagcttcccctgagtgttcaggaactatttcacagtctcttgtagaagccttccacctctcctggtccctatccaacctctccagtgtgggaactgtgtctatggcctcttctgagttcttaatatcatttgctgtcctctgtgtcccaattacagatttcttcaccaattaccgcccccctcagacacccacatcttcaaactacatagagtcccccagctttggccccatggctgactccctcttcagcagtgggatcctggccccagagatgccatctccagcctcgtcttcctcctcctcagggatgacccctcactcagggaatacacgcctacaggtgagcttatcctgctctgcaagtctggccagagcatgtgagagctagctcggcctctgcctggggccaggaggtaaaaagacatttactgggccaaccaggcagagggccccagagaacctaggcagcagagaaagaaaaccctacaaaacagaattacacgaactgaatgggggtggggggtgggggcagatggcagaagacgcacagaacagggcacaatccagaagaaagcttttagttctgttttgtttttgatacatggtctcagggccctgaatggccttggattcatagagatcaacctgtctttgcctcctgagtgcagccactaaaggtgtacaccatctgtacctgcctagtattctgagtcttagtgaatgacaggctagggaaagggcaagccagattggaagggccaggaatgtgtgcatggaaacagaatgggtgtgtgtctagacaactgtaaattggcatcttgtttcactaactcctgtgcccaattcagaagtcattcccctgccactgttaagtgtccattccagtttcatttctgttgttgtactaaaataccccaaggaaaaagcaacttagagggaagaaaggggttcatgttagcttgcaacctcaggttacagtccatctttgcggggtagtcaaggcaggaacctcagcaatcatagcacatctacagtcaggagcagagagaaatgattgcgtgcatgctcacctactcgcttacttatgttcagctccatttctctacttttacaccgttcaggatcctctgcttagggaatggtgccacccacagtgggctgggtcttcccatatctcctgacttctgacaattaagataatcccccaatagacatggctataggccaacccagcattagcagattctcactgagacccttcctataagaactcctggttgtcatgttgaaaggtaatcctaaccaccccagtgtcttcctcctatcattgaacaattcggctgcttttgcagctgatcttgagaggtctttctcctccactggacagcttgctggaaggggttgtcttggtcattattacatttgctgtttctagtcatttccatacacagattgccaatcaataaatggttgcccagtgagtgagtgattgagaatacaaagagacttatgcagctgtaaatggaaagatcgtggtagacaatggtgtgtgcatctgtgggtaagggaatgtaaagtagagtgggctggttcagaactgatgaactagtgggtggatggcctccgtgatgcatggctttgtgaatggctgtttagaactgatgaatgaattagtggtagttgacccagtgatgcttggctttgtgaatggatgagcgatttgttgccttcgtggctggtcaatgtacagatggtagatggatagacagatggagtatggatgggtggatgggagaacagagagttcagatggttggattgcttggtaaatggctcagctggcctgtcagctctctcatcaccaggacacacctagaaaaacaaccccagggaaggagttactaggctcttggcttcagaggttctagtctagcctgggggaggaagtgtaacagagctaggaaggaaagatggagaggttgggggtggggggagtgcctttactagaggacttccaccccccccaagttattccatttggaccccagactatgagaggagcattcatggcatgtctcccccatattagttacctctccccttgacaagatacccggcttaaggagcttactgagaggtttctttgggatcagttttgaaggataccatggagaaggtaagagagaagtaccacgaggcagctgctcacactgggtgagggagcagaggggagaaggctggtacccggctcacttgcttactttcatgtggtgatgacaatgatgatgacgatggcaatgagcaggaggaggaggaggagtgtgtgtgtgtgtgtgtgtgtgtgtgtgtgtgtgtgtgtgtgtgtccaggcgtgatgtagagcacatgcatgtcacggcatgtgtgtggaggtcagagaacagcttgaacgtagcagttttccccttgcaccatcctatcctgtgggtcccaggcactgaactcaagtcaaggcttggtggtaagtgttctacctactgagccactttgccagcccccatacttacatttttaaaattttattttgtgtgttttcacgtgtgtgtgcccatgtggaggtcagaggataaccttcaggagttagttctttcctttcccccatgtgagttcctgtggtcaaatcagcctatcaagcttggtgacaagggcttttaagtttctgagccatcacgctggcccacatcgtcttcagtctggtatcccacccctatggaatggttctgccacacccaggatgaatcttttctctctgttaaccctctcgggaaacatcatcagagatacacccaagaggtgggtctcactaatgccctacccatttcttaactgaatcacgttgacaatggcatttaaccagcacacctcaccttcttaagtctatcccttctggaaatgcctcaaagaaacaccagtgtacctcacccagacctagacattcctcattttagtccatcccagctggagttggagtagagagttgggtgagttcaggatgagagactaagttggtaaatgggtgcaaaaaaatggatggacagacagttgggtggatggacggatgcatggatggacaggtgggtggatggatggatagaaagatggattgacagatggacaagtggatggatggatggatggatggatggacaggtggatggatgggtaatacataagattaatgggtaagtcagtggggctgggcatatagatggatggatggatgggtgggtaggtggatggatagacaggtggatggatggatggatggatggatggatggatgagcaggtggatgggtgaatggatggacggacaggtggatgaatagatggacagatagatgaatggatggatggatgggtggatggacggatgaatggatggacaagtagatggatggattgatggatggatggatggatggatggatgggtgtatgggcaggtggatggatgggtgggtaggtggtggggtgcatgggtggataaagaatggatgaatggaaggtttgatgaatgagttagtgttttgatggatgaagaatagataattggaaggatagatggtgggtaattagtgaatgaattaatggatgactagatggctagataaatgaagactagattagtagctgtcttggacagattatttggtggctgagtggatcatctgtaagactgatccatgcctaatttgtgcacaagggttatgttggccacggctgtacatgaggttgtaatttctaaagtgtgcatgtatgcatatgtgtatgcatgtgcgtgcacatgtatggatcagttctcatctatacccagtccaatacatagccaggatagaggtttcctggtagttgcaagaaggtggctccatcccctgctctctgactatcatcctgtcttcctctgcccctccccctaggctcggaacagctgctctggacccttggatcccaacccctttctgagctctgaattccttcttcctgaagaccctaagaccaagatcccacctgctcctggaccgacccctctccttccattccccacccctgtcaaggtgcatggcttagagccctgcaccccctctcccttccccacaatggctccgcccccctctttgttgccagaagaatctctcctctctgccaggttcccattcacctcagcccctcctgctccaggagtgtctacgcttcctgcacccacaacctttgtccccaccccacagcccggccctggtcccgtccccttctctgtagaccatctgccccatgggtacctggaacccgtctttgggcctcacttcactgtgcctcagggtatgcaacccaggtgcaagccctcctccccatcccctgggggacaaaaagcaagtccacccaccttggcctctgccaccgccagtcccactgccactgccactgccagagacaacaacccctgtcttacacagctgctcagggcaggtgagatgggagaggcgaatggacgagatactgggaggaagcgactctgaaaggcatgggaaagcctgaaaggcagagggctggggtctcagggtggtcttgggagacagagaaggtggagtgatattagggatgtgagctatgtctgacaggggtgtgtgggctgctggggacaagaaggggtcttagttggtgatggacttgattgggccagtctagtggtcctcatagtcctgcctgtacccagccaagcctgagcaagcactggagcctccgactatgcccggtaccctcctccggcctccagagtccccggtaagatggtgggcactgggaggcgagggtcagtctagggcctttactccaactctctgcccttctccaccccagcaggatacagtttctgaaatcccccgtgcccgtgcctttttccccccaatcccggcccctacaccaccccggccacctccaggcccagccacattggctcctcccaggtccctggttgtccccaaagcagagcggctctcgcctccagcctccagcggtaaataggggctagaggggtcgtgggtcttaggggatagggaggggaaggatgaagggggctgtgaatggggtcagggcaaggggactttctcccttgtgtgtcggtctgtctgtgggtctgtccctgccacaggcagtgagcggcgactatcaggggatctcaactccatacaaccctcgggggcactgagtgtccacctgtctccccctcaaactgtcctaagccggggtcgtgtagacaacaacaaggtacgtgccgtcccctgacccctaagacgcaactggcttccaatccttccctgagccttcccacttagacccaccgcactttctgctttctaagatggagaaccgacgtatcacacacatctccgcggagcagaagaggcggttcaatattaagctaggattcgacaccctgcacggacttgtcagcacgctcagtgcccagcccagcctcaaggtgagcccccagccagcaatgcacactcccttagagtcgtggaacccctccttggaccaacacccaacaccagctcccagggcttgccaacacagaggcccacattggtggcgctcgcaggtgagcaaagcaaccacgcttcagaagacagcggagtacatcctgatgctgcagcaggaacgggcagctatgcaggaggaggcgcagcagctgcgggatgaaatagaggagctcaacgctgccatcaagtaagctggggtgaagcccagcctagaaaacctggagggaggggttagaggcgtggctccgaggtaccgcacctgcctagcctacttcattgaaggcctgggggcgtggcttagtgagagagtgctttcctaaatatctgctagtgagggtttgggggtgtggccccgtgaatagcgactgcccaaaatcctccactgaggagctccggatgtggtttgtcctagggtgcttacatagcatatttaagtaccgggtttcgtcctcagcaccttaaaaaaccaaacaacaacagcaaaagtcctcaggggctagggtgaaggagatgtgtctacattccctgaacggggactcttccccttcctagcttgtgccagcagcagctaccggccaccggggtgcccatcacacatcagcgctttgaccagatgcgggacatgtttgatgactatgtccggacgcgcacgctgcacaactggaagttctgggtggtatccttatttggactgatggcctgaggacagtggtggctgctgtagctacattcagtccagcctgggtctcagaccccagtctgagctgtagaacctgccccacccccacccccttccaccatgggctttggttctgccctctaccgtcagaccctagcattgcaagactgcgtggcaactgggactaggagctgagaggaagcaagagtaggggtaggaggttagggatgtccctgaggcagactggaaggatctaaagtctaagtcctttgaggctttgagaaggaagggtcaagttgtgtttgttttcctatactagcgatcgcacatgaccttcttcccaaccctcttttaacttttttattttgagacaaattctcactaagttattctggctagccttaaactcatgatcctcctgcctcagcctcctaaatggctgggattgtagacctgtgccattaggccagtattcaagtccttgactgagcctagagatgtgggacccactaatacagaatgagggttctggagggactaaggggagggactgagagatagagaggtgagtggataacccttggtgggagcgaaaaggcagagtggctctccacagggtccatggttcaacactgacaggcctttcttggccctggcttgggtcaggtgtcatggttctatgtggagtggtgggatcctgtctggaccactggtgggctccttgactccaaatccagttcagcatcctcatccgacctttatttgaatccttcaatgggatggtgtctaccgccagcttgcacagcctccgccagacctcactggcctggctggaacagtactgttccctacctgctctccggccaagtatgtgagataccttcctgccactaaattgtgaggctttgtagagcactggcctagactcccccagccccagggaggggctgggggcgtggctcagtggtagagcccctgcctagaatcccctagtgaggggctggggcatgacttagcagaagagcgtttacatcatcacaagtaagaccctgagttcaatttccagtgacacacatgtatacacatgtgcacacacatatgtacacatgtgcacgcacacatacttgtgggtttccagaacagagagttcaaacccagcctaggatttgagactgttttaccatctctcatcctgaatggtcctttagactctaaatccttctaaaggaaaaggtcctaggggagggccacctgtttagtgtagggtaaaaaatggggtcccatgtctgctcagggctaggaagtctgactgcaccatgaaccccatggaagcgccgggtgggcattggactttgggaagccatgggatcctgctctggggatgaggaagacacagtgtggggttcctgtggaactgtggaagtcggactccaactcttgggcactacagactgctgggcaacatggaagagccggttctggggtccctgggccgcacagaggccagggacaacaggttctcgctctcggacatcccagacactgctatatctcagaagagctgagaacaaagtggagaccctcgggtggactctggcccaggggccgaagggaaaagggtaggaggagagctctggcaggttaccgtggggaggagagtctggctagttcaggcagctggctcggtgggctgcttggtttggctggcagccgtggctggagtgcaggtaagcctcctgacaggaggatagacatgcagctctttagagggaaacctgcttcattgttccagcatagtgggttctgatgagcagggactgtccatggttttaaggtgtttattgtagaaaggcagagagagggagagagtaaagcagaggctggctatggccacatagagagaggggggaagggagggggagaaggagggctagaggcgagagaataagagcaagagggtaagagagggagcaaaacagagaggagggggcaagcagctccttttatagtggactgggctaacttgccgttgccaggtaactgtggggagaagcatacctggccaggtaactgtgggggtggagtctgaacagcctacatgactgatggccacagaattatggagttgagtccacgtgtcaggagcctatgtctgggagcatggcaaacaggccttctgtcccttgcggattaatctgctgggtctctggggtttaaacatagctcagcccagaaaacaggctccctttcacagtcctacagtttagctatctagtattgacttcatccctaactgtaaagacgtctgtcttagttagggtttctactgctatgaagagacaccatgaccaaggtaagtcttataaagaacgtttaactggggctggcttacagtttcagaggttcagtccattatcaaggcaggaagcatggcagcatctaggcaggcatggcactggaggagctgagagtgctccatcttcattcaaaggaagccagctaggaggagggtgtcaaagcccaccccacagtgacacacttcctccaacaaggccacacttcctaacagtgccactcactgggccaagagtattcaaatcaccacagcatctcactctctttttccagctgtcctgaattcccttcgccaactcagcacttccacaagcatcctgactgaccccagccttgtgcctgagcaagccacacgggcagtcacagagggcaccctggggagaccattataatcctggtgaaagtttccaagctccaggaacaacttgtgaacacgcttctaaaaatcctggctggccgtttccctgcagatctggaaggttccaagtgttgcactcaccacctccttggggatggagaaaaacctagttccattcctgcactgttgctaatgctatgatccctggtgactcatatcccatctttggacctccctttaataacagagggccccaaatgcattagcagctgcagcactggggacctaaacaggagctctgatgagagatgtcggtctgtttcctcacaacttcctgctttggataaagggtaccatccttagttcctgagcagagaggcagagcaggagatctcctctctttgcctttgcactagtgacatggggagctatagggtggcttcctgactgaggccactggggtctgatgccagaagagatcagggcaagggctagccagagcatggcaggcatcttgcatgaatgtagatgtgtgtgtgcgtatgtggattttataaaaaagaaaatgttcttcatcaataaaagcaaaaactgtctgcaagggttggtgttggtgtgactggcaatgtgtctcagtatgggactcagagctgccagcagtgaaaggtgacatagaagaccgatcctgtgggttataaacaacctttgagtacttttgatttgttatttttgtttttgttttttcgagacagggttttttttgtgtagccctggctgtcctggaacttactggataggccaggctggcctccaactcagagatccacccacccctgcctctggaatactggaattaaaggcatatgtcaccaccacccggccctttgagtacttttcaacttgcttgtgtgcagttgtgctcacctgtgcttgtgtgtggaggccgtcggctgacctgggtacctccttcgtgctctctgccttaattttaaaatgcttccgaatatattctagaactgggcatggtgatgcacacctttaatcccagtacttgggaggcagaggcaggtttttaatgagtgagaccagcctgttctgcatatggagttataggccagccagggccatatagtaagaccccgtgtgtacatacacacacatacacacacagagttacttttatatattattttgggggcagggtctctgtagtcttggctagcctagaacttgttatgtagaccaggttgggtttaaacaaccagatctttctgcctctgtctaaaaatatttaaaattacatttatttatatgtgtatgcacacatgcaaatat

SEQ ID NO: 19 - DNA, 20 bp, Artificial Sequence
  Oligomeric Compound
cgtgtgtctgtgctagtccc

SEQ ID NO: 20 - DNA, 20 bp, Artificial Sequence
  Oligomeric Compound
ggcaacgtgaacaggtccaa

SEQ ID NO: 21 - DNA, 18 bp, Artificial Sequence
  Oligomeric Compound
gcccattgctggacatgc

SEQ ID NO: 22 - DNA, 20 bp, Artificial Sequence
  Oligomeric Compound
agcccattgctggacatgca

SEQ ID NO: 23 - DNA, 20 bp, Artificial Sequence
  Oligomeric Compound
ttgtcccagtcccaggcctc

SEQ ID NO: 24 - DNA, 20 bp, Artificial Sequence
  Oligomeric Compound
ctttccgttggacccctggg

SEQ ID NO: 25 - DNA, 20 bp, Artificial Sequence
  Oligomeric Compound
gtgcgcgcgagcccgaaatc

SEQ ID NO: 26 - DNA, 20 bp, Artificial Sequence
  Oligomeric Compound
atccaagtgctactgtagta

SEQ ID NO: 27 - DNA, 20 bp, Artificial Sequence
  Oligomeric Compound
  misc_feature 1, 2, 3, 4, 5, 6, 7, 8, 9, 10, 11, 12, 13, 14, 15, 16,17, 18, 19, 20: n = A,T,C or G
nnnnnnnnnnnnnnnnnnnn

SEQ ID NO: 28 - DNA, 20 bp, Artificial Sequence
  Oligomeric Compound
gccctccatgctggcacagg

SEQ ID NO: 29 - DNA, 20 bp, Artificial Sequence
  Oligomeric Compound
agcaaaagatcaatccgtta

SEQ ID NO: 30 - DNA, 20 bp, Artificial Sequence
  Oligomeric Compound
tacagaaggctgggccttga

SEQ ID NO: 31 - DNA, 20 bp, Artificial Sequence
  Oligomeric Compound
atgcattctgcccccaagga

SEQ ID NO: 32 - DNA, 21 bp, Artificial Sequence
  Primer
caacggatttggtcgtattgg

SEQ ID NO: 33 - DNA, 26 bp, Artificial Sequence
  Primer
ggcaacaatatccactttaccagagt

SEQ ID NO: 34 - DNA, 21 bp, Artificial Sequence
  Probe
cgcctggtcaccagggctgct

SEQ ID NO: 35 - DNA, 19 bp, Artificial Sequence
  Primer
gaaggtgaaggtcggagtc

SEQ ID NO: 36 - DNA, 20 bp, Artificial Sequence
  Primer
gaagatggtgatgggatttc

SEQ ID NO: 37 - DNA, 20 bp, Artificial Sequence
  Probe
caagcttcccgttctcagcc

SEQ ID NO: 38 - DNA, 20 bp, Artificial Sequence
  Probe
tggaatcatattggaacatg

SEQ ID NO: 39 - DNA, 20 bp, Artificial Sequence
  Primer
ggcaaattcaacggcacagt

SEQ ID NO: 40 - DNA, 20 bp, Artificial Sequence
  Primer
gggtctcgctcctggaagat

SEQ ID NO: 41 - DNA, 27 bp, Artificial Sequence
  Probe
aaggccgagaatgggaagcttgtcatc

SEQ ID NO: 42 - DNA, 23 bp, Artificial Sequence
  Primer
tgttctagagacagccgcatctt

SEQ ID NO: 43 - DNA, 21 bp, Artificial Sequence
  Primer
caccgaccttcaccatcttgt

SEQ ID NO: 44 - DNA, 24 bp, Artificial Sequence
  Probe
ttgtgcagtgccagcctcgtctca

SEQ ID NO: 45 - DNA, 20 bp, Artificial Sequence
  Primer
caccgtgaccttgggtgact

SEQ ID NO: 46 - DNA, 21 bp, Artificial Sequence
  Primer
catccccattttgcagattga

SEQ ID NO: 47 - DNA, 24 bp, Artificial Sequence
  Probe
tccgctgtctttggaccgctgtgt

SEQ ID NO: 48 - DNA, 24 bp, Artificial Sequence
  Primer
cgggacatgtttgatgactatgtc

SEQ ID NO: 49 - DNA, 21 bp, Artificial Sequence
  Primer
tcggatgaggatgctgaacac

SEQ ID NO: 50 - DNA, 24 bp, Artificial Sequence
  Probe
cacgctgcacaactggaagttctg

SEQ ID NO: 51 - DNA, 20 bp, Artificial Sequence
  Oligomeric Compound
ggcaagactgtcaccccctc

SEQ ID NO: 52 - DNA, 20 bp, Artificial Sequence
  Oligomeric Compound
acgctgtggccacacggtgg

SEQ ID NO: 53 - DNA, 20 bp, Artificial Sequence
  Oligomeric Compound
ggtaaagaaatactggatat

SEQ ID NO: 54 - DNA, 20 bp, Artificial Sequence
  Oligomeric Compound
ttcactgcctgtggtaggga

SEQ ID NO: 55 - DNA, 20 bp, Artificial Sequence
  Oligomeric Compound
tggtccctgctccgcgcagc

SEQ ID NO: 56 - DNA, 20 bp, Artificial Sequence
  Oligomeric Compound
acccgcgggacctgcaagcc

SEQ ID NO: 57 - DNA, 20 bp, Artificial Sequence
  Oligomeric Compound
agactcgggtcctccgagtc

SEQ ID NO: 58 - DNA, 20 bp, Artificial Sequence
  Oligomeric Compound
ggctcaagcactcgaagagg

SEQ ID NO: 59 - DNA, 20 bp, Artificial Sequence
  Oligomeric Compound
cagagcagcttgaggccttt

SEQ ID NO: 60 - DNA, 20 bp, Artificial Sequence
  Oligomeric Compound
cgcttcacatactggatata

SEQ ID NO: 61 - DNA, 20 bp, Artificial Sequence
  Oligomeric Compound
gtcacgaagccacacacggg

SEQ ID NO: 62 - DNA, 20 bp, Artificial Sequence
  Oligomeric Compound
agccgcttcttgtagtagat

SEQ ID NO: 63 - DNA, 20 bp, Artificial Sequence
  Oligomeric Compound
tacggagccgcttcttgtag

SEQ ID NO: 64 - DNA, 20 bp, Artificial Sequence
  Oligomeric Compound
ggtcatcttccctgctgggc

SEQ ID NO: 65 - DNA, 20 bp, Artificial Sequence
  Oligomeric Compound
gggaccacactggagaagag

SEQ ID NO: 66 - DNA, 20 bp, Artificial Sequence
  Oligomeric Compound
aggagctgccgcccacccgg

SEQ ID NO: 67 - DNA, 20 bp, Artificial Sequence
  Oligomeric Compound
gtgtctgagatgtcggacaa

SEQ ID NO: 68 - DNA, 20 bp, Artificial Sequence
  Oligomeric Compound
gggccggactgagtcatggt

SEQ ID NO: 69 - DNA, 20 bp, Artificial Sequence
  Oligomeric Compound
aggcatcctcaggcggcagc

SEQ ID NO: 70 - DNA, 20 bp, Artificial Sequence
  Oligomeric Compound
tgccgacgtaggcatcctca

SEQ ID NO: 71 - DNA, 20 bp, Artificial Sequence
  Oligomeric Compound
tccatgaagtcatccaggct

SEQ ID NO: 72 - DNA, 20 bp, Artificial Sequence
  Oligomeric Compound
cactgctgaagagggagtca

SEQ ID NO: 73 - DNA, 20 bp, Artificial Sequence
  Oligomeric Compound
acggctgtgtccagagaggt

SEQ ID NO: 74 - DNA, 20 bp, Artificial Sequence
  Oligomeric Compound
agagaagagaggctcttcct

SEQ ID NO: 75 - DNA, 20 bp, Artificial Sequence
  Oligomeric Compound
ctgtgagcagctgtgtgagg

SEQ ID NO: 76 - DNA, 20 bp, Artificial Sequence
  Oligomeric Compound
gaggtcccctgacagccgcc

SEQ ID NO: 77 - DNA, 20 bp, Artificial Sequence
  Oligomeric Compound
agtcccagggcctggcatgg

SEQ ID NO: 78 - DNA, 20 bp, Artificial Sequence
  Oligomeric Compound
gcggagatgtgtgtgatacg

SEQ ID NO: 79 - DNA, 20 bp, Artificial Sequence
  Oligomeric Compound
gctccgcggagatgtgtgtg

SEQ ID NO: 80 - DNA, 20 bp, Artificial Sequence
  Oligomeric Compound
aaaccccagcttgatgttga

SEQ ID NO: 81 - DNA, 20 bp, Artificial Sequence
  Oligomeric Compound
tcaccttgaggctgggctgg

SEQ ID NO: 82 - DNA, 20 bp, Artificial Sequence
  Oligomeric Compound
tttgctcaccttgaggctgg

SEQ ID NO: 83 - DNA, 20 bp, Artificial Sequence
  Oligomeric Compound
tggtagctttgctcaccttg

SEQ ID NO: 84 - DNA, 20 bp, Artificial Sequence
  Oligomeric Compound
cagctgtcttctgcagcgtg

SEQ ID NO: 85 - DNA, 20 bp, Artificial Sequence
  Oligomeric Compound
gtcatcaaacatgtctcgca

SEQ ID NO: 86 - DNA, 20 bp, Artificial Sequence
  Oligomeric Compound
gccgtggacaccatcccgtt

SEQ ID NO: 87 - DNA, 20 bp, Artificial Sequence
  Oligomeric Compound
tgcccagctggcgtagggag

SEQ ID NO: 88 - DNA, 20 bp, Artificial Sequence
  Oligomeric Compound
cccgggtcggtcaggatact

SEQ ID NO: 89 - DNA, 20 bp, Artificial Sequence
  Oligomeric Compound
ataaaggtttgccaagggtg

SEQ ID NO: 90 - DNA, 20 bp, Artificial Sequence
  Oligomeric Compound
tggccaggactataaaggtt

SEQ ID NO: 91 - DNA, 20 bp, Artificial Sequence
  Oligomeric Compound
gcccagagatgatccctgga

SEQ ID NO: 92 - DNA, 20 bp, Artificial Sequence
  Oligomeric Compound
gggagtgcccagagatgatc

SEQ ID NO: 93 - DNA, 20 bp, Artificial Sequence
  Oligomeric Compound
ttgacctccaggaggtggca

SEQ ID NO: 94 - DNA, 20 bp, Artificial Sequence
  Oligomeric Compound
gggactctgctcttcttgac

SEQ ID NO: 95 - DNA, 20 bp, Artificial Sequence
  Oligomeric Compound
ggacgagtcacccaaggtca

SEQ ID NO: 96 - DNA, 20 bp, Artificial Sequence
  Oligomeric Compound
ccccattttgcagattgaaa

SEQ ID NO: 97 - DNA, 20 bp, Artificial Sequence
  Oligomeric Compound
tctgctgattgaaccttccc

SEQ ID NO: 98 - DNA, 20 bp, Artificial Sequence
  Oligomeric Compound
gggtcatctgctgattgaac

SEQ ID NO: 99 - DNA, 20 bp, Artificial Sequence
  Oligomeric Compound
gacagataaacagcatcctc

SEQ ID NO: 100 - DNA, 20 bp, Artificial Sequence
  Oligomeric Compound
gaggtctgtgccccacctgt

SEQ ID NO: 101 - DNA, 20 bp, Artificial Sequence
  Oligomeric Compound
tttctgcttctctgctcagg

SEQ ID NO: 102 - DNA, 20 bp, Artificial Sequence
  Oligomeric Compound
gagagagggaacctcctttt

SEQ ID NO: 103 - DNA, 20 bp, Artificial Sequence
  Oligomeric Compound
agcagtgaaggagcagagag

SEQ ID NO: 104 - DNA, 20 bp, Artificial Sequence
  Oligomeric Compound
gtctcctgggatcaggccct

SEQ ID NO: 105 - DNA, 20 bp, Artificial Sequence
  Oligomeric Compound
ccctgctgtggtcactctgg

SEQ ID NO: 106 - DNA, 20 bp, Artificial Sequence
  Oligomeric Compound
ttgcttttattggtcaagaa

SEQ ID NO: 107 - DNA, 20 bp, Artificial Sequence
  Oligomeric Compound
gcatcaccacctcgatgcgc

SEQ ID NO: 108 - DNA, 20 bp, Artificial Sequence
  Oligomeric Compound
tggatcatgtcagcattgcc

SEQ ID NO: 109 - DNA, 20 bp, Artificial Sequence
  Oligomeric Compound
ccgcacgctccttgaggctg

SEQ ID NO: 110 - DNA, 20 bp, Artificial Sequence
  Oligomeric Compound
aacttccagttgtgcagcgt

SEQ ID NO: 111 - DNA, 20 bp, Artificial Sequence
  Oligomeric Compound
cccagaacttccagttgtgc

SEQ ID NO: 112 - DNA, 20 bp, Artificial Sequence
  Oligomeric Compound
tgctgaacacccagaacttc

SEQ ID NO: 113 - DNA, 20 bp, Artificial Sequence
  Oligomeric Compound
gaggatgctgaacacccaga

SEQ ID NO: 114 - DNA, 20 bp, Artificial Sequence
  Oligomeric Compound
cggatgaggatgctgaacac

SEQ ID NO: 115 - DNA, 20 bp, Artificial Sequence
  Oligomeric Compound
caggccagtgaggtctggcg

SEQ ID NO: 116 - DNA, 20 bp, Artificial Sequence
  Oligomeric Compound
ccagccaggccagtgaggtc

SEQ ID NO: 117 - DNA, 20 bp, Artificial Sequence
  Oligomeric Compound
aggacagttggccggagagc

SEQ ID NO: 118 - DNA, 20 bp, Artificial Sequence
  Oligomeric Compound
gcccgtgtggcttgctcagg

SEQ ID NO: 119 - DNA, 20 bp, Artificial Sequence
  Oligomeric Compound
tgactgcccgtgtggcttgc

SEQ ID NO: 120 - DNA, 20 bp, Artificial Sequence
  Oligomeric Compound
ctctgtgactgcccgtgtgg

SEQ ID NO: 121 - DNA, 20 bp, Artificial Sequence
  Oligomeric Compound
gtgccctctgtgactgcccg

SEQ ID NO: 122 - DNA, 20 bp, Artificial Sequence
  Oligomeric Compound
gcagacagtttttgctttta

SEQ ID NO: 123 - DNA, 20 bp, Artificial Sequence
  Oligomeric Compound
ggttcttgttgctgtctgga

SEQ ID NO: 124 - DNA, 20 bp, Artificial Sequence
  Oligomeric Compound
gacagccgccgttcactgcc

SEQ ID NO: 125 - DNA, 20 bp, Artificial Sequence
  Oligomeric Compound
cagatcccaaaggaaggccg

SEQ ID NO: 126 - DNA, 20 bp, Artificial Sequence
  Oligomeric Compound
tgccttccgcctagggagac

SEQ ID NO: 127 - DNA, 20 bp, Artificial Sequence
  Oligomeric Compound
gcaacagcagttagggccag

SEQ ID NO: 128 - skipped

SEQ ID NO: 129 - DNA, 20 bp, Artificial Sequence
  Oligomeric Compound
tcacggatagatccgccagc

SEQ ID NO: 130 - DNA, 20 bp, Artificial Sequence
  Oligomeric Compound
accatgaagtgtccgctgtg

SEQ ID NO: 131 - DNA, 20 bp, Artificial Sequence
  Oligomeric Compound
aagttcttccacttgggaga

SEQ ID NO: 132 - DNA, 20 bp, Artificial Sequence
  Oligomeric Compound
atagcaacttgaggcctttg

SEQ ID NO: 133 - DNA, 20 bp, Artificial Sequence
  Oligomeric Compound
atgtaccaggctctccagat

SEQ ID NO: 134 - DNA, 20 bp, Artificial Sequence
  Oligomeric Compound
tcctccgttgcacatactga

SEQ ID NO: 135 - DNA, 20 bp, Artificial Sequence
  Oligomeric Compound
caggtttccggtgctcatct

SEQ ID NO: 136 - DNA, 20 bp, Artificial Sequence
  Oligomeric Compound
acttacggagccgctttttg

SEQ ID NO: 137 - DNA, 20 bp, Artificial Sequence
  Oligomeric Compound
ccaggaaatccccttccctg

SEQ ID NO: 138 - DNA, 20 bp, Artificial Sequence
  Oligomeric Compound
accttccacctgcttgggag

SEQ ID NO: 139 - DNA, 20 bp, Artificial Sequence
  Oligomeric Compound
ccacgctggagaagagctgt

SEQ ID NO: 140 - DNA, 20 bp, Artificial Sequence
  Oligomeric Compound
aagctgccggcccccaggct

SEQ ID NO: 141 - DNA, 20 bp, Artificial Sequence
  Oligomeric Compound
gagtgtgtcggagatatcgg

SEQ ID NO: 142 - DNA, 20 bp, Artificial Sequence
  Oligomeric Compound
ctgggctgtgtcatggtgaa

SEQ ID NO: 143 - DNA, 20 bp, Artificial Sequence
  Oligomeric Compound
aatctgatatctccatgaag

SEQ ID NO: 144 - DNA, 20 bp, Artificial Sequence
  Oligomeric Compound
tgttccgagcctgtaggcgt

SEQ ID NO: 145 - DNA, 20 bp, Artificial Sequence
  Oligomeric Compound
ttcaggaagaaggaattcag

SEQ ID NO: 146 - DNA, 20 bp, Artificial Sequence
  Oligomeric Compound
cagggctctaagccatgcac

SEQ ID NO: 147 - DNA, 20 bp, Artificial Sequence
  Oligomeric Compound
gccctgagcagctgtgtaag

SEQ ID NO: 148 - DNA, 20 bp, Artificial Sequence
  Oligomeric Compound
cgctcactgccgctggaggc

SEQ ID NO: 149 - DNA, 20 bp, Artificial Sequence
  Oligomeric Compound
ggcttaggacagtttgaggg

SEQ ID NO: 150 - DNA, 20 bp, Artificial Sequence
  Oligomeric Compound
acacgaccccggcttaggac

SEQ ID NO: 151 - DNA, 20 bp, Artificial Sequence
  Oligomeric Compound
gttgttgtctacacgacccc

SEQ ID NO: 152 - DNA, 20 bp, Artificial Sequence
  Oligomeric Compound
aatcctagcttaatattgaa

SEQ ID NO: 153 - DNA, 20 bp, Artificial Sequence
  Oligomeric Compound
tgagcgtgctgacaagtccg

SEQ ID NO: 154 - DNA, 20 bp, Artificial Sequence
  Oligomeric Compound
gcgtggttgctttgctcacc

SEQ ID NO: 155 - DNA, 20 bp, Artificial Sequence
  Oligomeric Compound
gtcttctgaagcgtggttgc

SEQ ID NO: 156 - DNA, 20 bp, Artificial Sequence
  Oligomeric Compound
gagctcctctatttcatccc

SEQ ID NO: 157 - DNA, 20 bp, Artificial Sequence
  Oligomeric Compound
gctgctggcacaagttgatg

SEQ ID NO: 158 - DNA, 20 bp, Artificial Sequence
  Oligomeric Compound
gccggtagctgctgctggca

SEQ ID NO: 159 - DNA, 20 bp, Artificial Sequence
  Oligomeric Compound
caccccggtggccggtagct

SEQ ID NO: 160 - DNA, 20 bp, Artificial Sequence
  Oligomeric Compound
gtgtgatgggcaccccggtg

SEQ ID NO: 161 - DNA, 20 bp, Artificial Sequence
  Oligomeric Compound
cccgcatctggtcaaagcgc

SEQ ID NO: 162 - DNA, 20 bp, Artificial Sequence
  Oligomeric Compound
tcaaacatgtcccgcatctg

SEQ ID NO: 163 - DNA, 20 bp, Artificial Sequence
  Oligomeric Compound
gacatagtcatcaaacatgt

SEQ ID NO: 164 - DNA, 20 bp, Artificial Sequence
  Oligomeric Compound
gacaccatcccattgaagga

SEQ ID NO: 165 - DNA, 20 bp, Artificial Sequence
  Oligomeric Compound
tggcggaggctgtgcaagct

SEQ ID NO: 166 - DNA, 20 bp, Artificial Sequence
  Oligomeric Compound
cgaagggaattcaggacagt

SEQ ID NO: 167 - DNA, 20 bp, Artificial Sequence
  Oligomeric Compound
caccaggattataatggtct

SEQ ID NO: 168 - DNA, 20 bp, Artificial Sequence
  Oligomeric Compound
tgttcctggagcttggaaac

SEQ ID NO: 169 - DNA, 20 bp, Artificial Sequence
  Oligomeric Compound
caagttgttcctggagcttg

SEQ ID NO: 170 - DNA, 20 bp, Artificial Sequence
  Oligomeric Compound
atctctcatcagagctcctg

SEQ ID NO: 171 - DNA, 20 bp, Artificial Sequence
  Oligomeric Compound
ctacattcatgcaagatgcc

SEQ ID NO: 172 - DNA, 20 bp, Artificial Sequence
  Oligomeric Compound
cacatctacattcatgcaag

SEQ ID NO: 173 - DNA, 20 bp, Artificial Sequence
  Oligomeric Compound
ttgcttttattgatgaagaa

SEQ ID NO: 174 - DNA, 20 bp, Artificial Sequence
  Oligomeric Compound
atgaggaccactgccctgag

SEQ ID NO: 175 - DNA, 20 bp, Artificial Sequence
  Oligomeric Compound
gctcaggcttggctgggtac

SEQ ID NO: 176 - DNA, 20 bp, Artificial Sequence
  Oligomeric Compound
ttggcaagcccttgaggctg

SEQ ID NO: 177 - DNA, 20 bp, Artificial Sequence
  Oligomeric Compound
gccctgggagcttgaggctg

SEQ ID NO: 178 - DNA, 20 bp, Artificial Sequence
  Oligomeric Compound
gtgggcctctgtgttggcaa

SEQ ID NO: 179 - DNA, 20 bp, Artificial Sequence
  Oligomeric Compound
tctaagggagtgtgcattgc

SEQ ID NO: 180 - DNA, 20 bp, Artificial Sequence
  Oligomeric Compound
ttagccatccagaaagtcag

SEQ ID NO: 181 - DNA, 20 bp, Artificial Sequence
  Oligomeric Compound
agactgacggtgctgggtag

SEQ ID NO: 182 - DNA, 20 bp, Artificial Sequence
  Oligomeric Compound
ggagccactgcgtggatgct

SEQ ID NO: 183 - DNA, 20 bp, Artificial Sequence
  Oligomeric Compound
aaagagaagaggagctggag

SEQ ID NO: 184 - DNA, 20 bp, Artificial Sequence
  Oligomeric Compound
ctccagctacctcaggtttc

SEQ ID NO: 185 - DNA, 20 bp, Artificial Sequence
  Oligomeric Compound
gaacactcacctgcttggga

SEQ ID NO: 186 - DNA, 20 bp, Artificial Sequence
  Oligomeric Compound
atgaggaccactagactggc

SEQ ID NO: 187 - DNA, 20 bp, Artificial Sequence
  Oligomeric Compound
ccctatttaccgctggaggc

SEQ ID NO: 188 - DNA, 20 bp, Artificial Sequence
  Oligomeric Compound
ttggcaagccctgggagctg

SEQ ID NO: 189 - DNA, 20 bp, Artificial Sequence
  Oligomeric Compound
cccagcttacttgatggcag

SEQ ID NO: 190 - DNA, 15 bp, Artificial Sequence
  Oligomeric Compound
gaggtctgtgcccca

SEQ ID NO: 191 - DNA, 15 bp, Artificial Sequence
  Oligomeric Compound
gtctgtgccccacct

SEQ ID NO: 192 - DNA, 13 bp, Artificial Sequence
  Oligomeric Compound
aggtctgtgcccc

SEQ ID NO: 193 - DNA, 24 bp, Artificial Sequence
  Oligomeric Compound
gaggtctgtgccccacctgtcggg

SEQ ID NO: 194 - DNA, 14 bp, Artificial Sequence
  Oligomeric Compound
ccccacctgtcggg

SEQ ID NO: 195 - DNA, 35 bp, Artificial Sequence
  Oligomeric Compound
aacagaggtctgtgccccacctgtcggggagcaag

SEQ ID NO: 196 - DNA, 28 bp, Artificial Sequence
  Oligomeric Compound
aggtctgtgccccacctgtcggggagca

SEQ ID NO: 197 - DNA, 23 bp, Artificial Sequence
  Oligomeric Compound
tgccccacctgtcggggagcaag

SEQ ID NO: 198 - DNA, 20 bp, Artificial Sequence
  Oligomeric Compound
tggccagcactataaaggtt

SEQ ID NO: 199 - DNA, 20 bp, Artificial Sequence
  Oligomeric Compound
tggccaggactatacaggtt

SEQ ID NO: 200 - DNA, 20 bp, Artificial Sequence
  Oligomeric Compound
tggccaggactataaagagt

SEQ ID NO: 201 - DNA, 20 bp, Artificial Sequence
  Oligomeric Compound
aggccaggaatataaaggtt

SEQ ID NO: 202 - DNA, 20 bp, Artificial Sequence
  Oligomeric Compound
tagccaggattatcaaggtt

(19) United States
(12) Patent Application Publication
(10) Pub. No.: US 2009/0209625 A1
(43) Pub. Date: Aug. 20, 2009

(54) MODULATION OF CHREBP EXPRESSION

(76) Inventors: Sanjay Bhanot, Carlsbad, CA (US); Kenneth W. Dobie, Del Mar, CA (US); Susan F. Murray, Carlsbad, CA (US)

Correspondence Address: McDermott Will & Emery, 11682 EL CAMINO REAL, SUITE 400, SAN DIEGO, CA, 92130-2047, US

(21) Appl. No.: 12/301,460

(22) PCT Filed: May 23, 2007
(86) PCT No.: PCT/US07/69587

§ 371 Date: Mar. 11, 2009

Related U.S. Application Data
(60) Provisional application No. 60/802,948, filed on May 23, 2006.

(51) Int. Cl. A61K 48/00; C07H 21/00; C12N 5/00; A61P 3/04; A61P 3/10

(52) U.S. Cl. 514/44 A; 536/23.1; 435/375; 514/81

(57) ABSTRACT

Disclosed herein are compounds, compositions, and methods for modulating the expression of ChREBP in a cell, tissue or animal. Also provided are methods of target validation. Also provided are uses of disclosed compounds and compositions in the manufacture of a medicament for treatment of diseases and conditions.

### INCORPORATION OF SEQUENCE LISTING

The present application is being filed along with a Sequence Listing in electronic format. The Sequence Listing is provided as a file entitled RTS0750WOSEQ.TXT, created on May 23, 2007 which is 158 Kb in size. The information in the electronic format of the sequence listing is incorporated herein by reference in its entirety.

### FIELD OF THE INVENTION

Disclosed herein are compounds, compositions and methods for modulating the expression of ChREBP in a cell, tissue or animal.

### BACKGROUND OF THE INVENTION

Excess dietary carbohydrates induce the expression of genes encoding enzymes involved in the metabolic conversion of glucose to triglycerides in the liver. This response includes transcriptional activation of genes for enzymes involved in the breakdown of glucose, or glycolysis, such as L-type pyruvate kinase (LPK) and phosphofructokinase (PFK), and of those involved in de novo triglyceride synthesis, or lipogenesis, such as acetyl CoA carboxylase (ACC) and fatty acid synthase (FAS). Genes for many of these enzymes have a region within their promoters known as the carbohydrate response element or ChRE. ChRE consists of two 5′-CACGTG-type E-box motifs separated by 5 base pairs to which glucose responsive transcription factors can bind to induce activation (Uyeda et al., Biochem. Pharmacol., 2002, 63, 2075-2080).

Carbohydrate responsive element-binding protein (also known as ChREBP, Williams Beuren Syndrome Chromosome Region 14 or WBSCR14, Williams Syndrome basic helix-loop-helix protein or WS-bHLH, Mondo Family member B or MondoB, and Mlx interactor or Mio), was first identified as a transcription factor that recognizes the ChRE within the promoter of the LPK gene and is responsive to diet (i.e., activated by a high carbohydrate diet, and inhibited by high fat or starvation) (Yamashita et al., Proc. Natl. Acad. Sci. USA, 2001, 98, 9116-9121).

The gene for ChREBP was mapped to 7q11.23, a region deleted in Williams-Beuren Syndrome (WBS). WBS is a neurodevelopment disorder characterized by congenital heart and vascular disease, hypertension, infantile hypercalcemia, dysmorphic facial features, mental retardation, premature aging of the skin, growth retardation, and unique cognitive and personality profiles (Francke, Hum. Mol. Genet., 1999, 8, 1947-1954; Meng et al., Hum. Genet., 1998, 103, 590-599).

ChREBP is expressed in multiple tissues, predominantly in adult liver and at late stages of fetal development in both human and mouse (de Luis et al., Eur. J. Hum. Genet., 2000, 8, 215-222). ChREBP is also expressed in regions of the brain, heart, kidney, the intestinal tract and adipose tissue (Cairo et al., Hum. Mol. Genet., 2001, 10, 617-627; Letexier et al., J Lipid Res., 2003, 44, 2127-2134).

ChREBP is a member of the basic helix-loop-helix leucine zipper (bHLHZip) containing Myc/Max/Mad superfamily of transcription factors known to form dimers and recognize E-box motifs within their target promoters. ChREBP forms heterodimers with the bHLH-Zip interacting Max-like protein X (Mlx) in regulating the expression of glucose responsive genes. CHREBP contains several other domains including a bipartite nuclear localization signal (NLS) near the N-terminus, polyproline domains, and a leucine-zipper-like domain. Mutation analysis of mouse ChREBP showed that both the NLS and the bHLHZip domains were essential to ChREBP-mediated transcription (Cairo et al., Hum. Mol. Genet., 2001, 10, 617-627; Stoeckman et al., J. Biol. Chem., 2004).

Regulation of ChREBP activity is accomplished by phosphorylation/dephosphorylation of the protein, which serves to inactivate/activate it. An example of activation following dephosphorylation includes the response to elevated glucose. High glucose acts through xylulose 5-phosphate to stimulate a protein phosphatase, such as protein phosphatase 2A, to dephosphorylate ChREBP. ChREBP is subsequently translocated from the cytosol to the nucleus, where further dephosphorylation leads to DNA binding and activation of LPK transcription (Kabashima et al., Proc. Natl. Acad. Sci. USA, 2003, 100, 5107-5112; Kawaguchi et al., Proc. Natl. Acad. Sci. USA, 2001, 98, 13710-13715).

ChREBP activity can be inhibited in response to fatty acids. ChREBP is inactivated by cAMP-dependent protein kinase (PKA)- and AMP-activated protein kinase (AMPK)-mediated phosphorylation which deactivate nuclear import of ChREBP, and also dissociate ChREBP from DNA and inactivate LPK transcription (Ferre et al., Biochem. Soc. Trans., 2003, 31, 220-223; Kawaguchi et al., J. Biol. Chem., 2002, 277, 3829-3835). Together these data illustrate multiple mechanisms for regulation of ChREBP, and consequently, genes harboring the ChRE within their promoters.

In hepatocytes where small interfering RNA was used to reduce ChREBP expression, the induction of glycolytic and lipogenic genes was also inhibited. Furthermore, ChREBP was found to mediate the synergistic action of an insulin-responsive transcription factor, also known as the sterol regulatory element binding protein-1c (SREBP-1c), and glucokinase to activate LPK, FAS and ACC gene expression (Dentin et al., J. Biol. Chem., 2004). These data further demonstrate that ChREBP mediates the dietary induction of glycolytic and lipogenic gene expression in the liver.

Regulation of carbohydrate metabolism by ChREBP is critical to maintaining a balance between nutrient utilization and storage. Failure to regulate the activation of genes involved in glucose metabolism and fat storage can lead to diseased states such as diabetes, obesity, and hypertension (Uyeda et al., Biochem. Pharmacol., 2002, 63, 2075-2080). Furthermore, proper function of ChREBP has been suggested to be involved in growth control and may also contribute to some aspects of the WBS pathology (Cairo et al., Hum. Mol. Genet., 2001, 10, 617-627).

The US pre-grant publication 20030124590 discloses antisense primers designed to rat ChREBP (Uyeda, 2003).

The European patent application EP 1293569 discloses primers, probes and antisense polynucleotides to a group of polynucleotide sequences, including ChREBP (Isogai et al., 2004).

The pharmacological modulation of ChREBP activity and/or expression may therefore be an appropriate point of therapeutic intervention in diseases or conditions such as obesity, diabetes, or vascular disease. Furthermore, agents that modulate carbohydrate metabolism, lipogenesis and/or glycolysis may be of use therapeutically.

Currently, there are no known therapeutic agents which effectively modulate the synthesis of ChREBP. There remains a long felt need for additional agents capable of effectively modulating CHREBP expression.

Antisense technology is an effective means for reducing the expression of ChREBP and is uniquely useful in a number of therapeutic, diagnostic, and research applications.

Disclosed herein are antisense compounds useful for modulating gene expression and associated pathways via antisense mechanisms of action such as RNaseH, RNAi and dsRNA enzymes, as well as other antisense mechanisms based on target degradation or target occupancy. One of ordinary skill in the art, once armed with this disclosure will be able, without undue experimentation, to identify, prepare and exploit antisense compounds for these uses.

### SUMMARY OF THE INVENTION

The present invention is directed to oligomeric compounds specifically hybridizable with a nucleic acid molecule encoding ChREBP and which modulate the expression of ChREBP. Contemplated and provided herein are oligomeric compounds comprising sequences 13 to 80, 13 to 50 and 13 to 30 nucleotides in length. Also provided are oligomeric compounds comprising at least one chemical modification selected from a modified internucleoside linkage, a modified nucleobase, or a modified sugar. Further provided are modified oligomeric compounds in which the modified internucleoside linkage is a phosphorothioate, the modified nucleobase is a 5-methylcytosine and the modified sugar moiety is 2′-O-(2-methoxyethyl). Provided herein are chimeric oligonucleotides, including chimeric oligonucleotides comprising a deoxy nucleotide region flanked on each of the 5′ and 3′ ends with at least one 2′-O-(2-methoxyethyl) nucleotide. Further provided are chimeric oligonucleotides comprising ten deoxynucleotides and flanked on both the 5′ and 3′ ends with five 2′-O-(2-methoxyethyl) nucleotides wherein each internucleoside linkage is a phosphorothioate.

In one aspect there are provided chimeric oligonucleotides that are targeted to nucleic acids encoding ChREBP as modulators of ChREBP expression. The nucleic acids encoding ChREBP have a sequence that is substantially similar to one or more of SEQ ID NOS: 1 to 10 or 11 to 18, herein.

In another aspect, active target segments of the nucleic acids encoding ChREBP have been discovered. Active target segments are segments of nucleic acids encoding ChREBP (hereinafter “target nucleic acid.”) that are accessible to antisense hybridization and so are suitable for antisense modulation. The active target segments have been discovered herein using empirical data that is presented below, wherein at least two chimeric oligonucleotides are shown to hybridize within the active target segment and reduce expression of the target nucleic acid. Hereinafter, “active antisense compound” refers to a chimeric oligonucleotide that hybridizes with the target nucleic acid to reduce its expression. The active antisense compounds hybridizing within the active target segment are preferably separated by about 60 nucleobases on the target nucleic acid. Using the information provided herein regarding active target segments, additional active antisense compounds can be designed to target the active target segment and modulate expression of the target nucleic acid.

Further provided are methods of modulating the expression of ChREBP in cells, tissues or animals comprising contacting said cells, tissues or animals with one or more of the compounds or compositions of the present invention. For example, in one embodiment, the compounds or compositions of the present invention can be used to inhibit the expression of ChREBP in cells, tissues or animals.

In one embodiment, the present invention provides methods of lowering blood glucose, cholesterol and triglyceride levels. In another embodiment, the present invention provides methods of improving insulin sensitivity.

In one embodiment, the present invention provides methods of improving hyperlipidemia. In another embodiment, the hyperlipidemia is associated with metabolic syndrome.

In other embodiments, the present invention is directed to methods of preventing, ameliorating or lessening the severity of a disease or condition in an animal comprising contacting said animal with an effective amount of an oligomeric compound of the invention. In other embodiments, the methods of the present invention inhibit expression of ChREBP. In an additional embodiment of the methods of the present invention, the ameliorating or lessening of the severity of the disease or condition of an animal is measured by one or more physical indicators of said disease or condition. In one embodiment, the animal is a primate. In some embodiments, the disease or conditions include, but are not limited to, obesity, diabetes, insulin resistance, insulin deficiency, hypercholesterolemia, hyperglycemia, hyperlipidemia, hypertriglyceridemia, hyperfattyacidemia and liver steatosis. In some embodiments, the diabetes is type II diabetes. In some embodiments the steatosis is steatohepatitis or non-alcoholic steatohepatitis. In another embodiment, the disease or condition is metabolic syndrome. In another embodiment, the disease or condition is a cardiovascular disease. In another embodiment, the cardiovascular disease is coronary heart disease.

### DETAILED DESCRIPTION

Disclosed herein are compositions and methods for modulating the expression of ChREBP (also known as Williams-Beuren syndrome chromosome region 14; Carbohydrate Response Element Bind Prot; Carbohydrate response element-binding protein; MIO; MONDO FAMILY, MEMBER B; MONDOB; WBSCR14; WBSCR14; WS basic-helix-loop-helix leucine zipper protein; WS-BHLH; WS-bHLH; Williams-Beuren syndrome chromosome region 14 protein; and putative hepatic transcription factor). Listed in Table 1 are GENBANK® accession numbers of sequences useful for design of oligomeric compounds targeted to ChREBP. Oligomeric compounds of the invention include oligomeric compounds which hybridize with one or more target nucleic acid molecules shown in Table 1, as well as oligomeric compounds which hybridize to other nucleic acid molecules encoding ChREBP having a sequence that is substantially similar to one or more of SEQ ID NOS 1 to 10 or 11 to 18. The oligomeric compounds may target any region, segment, or site of nucleic acid molecules which encode ChREBP. Suitable target regions, segments, and sites include, but are not limited to, the 5′UTR, the start codon, the stop codon, the coding region, the 3′UTR, the 5′cap region, introns, exons, intron-exon junctions, exon-intron junctions, and exon-exon junctions.

TABLE 1
Gene Targets

| | | | SEQ ID |
|---|---|---|---|
| Species | Genbank # | Entry Date | NO |
| Human | BC012925.1 | Aug. 22, 2001 | 1 |
| Human | BG396554.1 | Mar. 5, 2001 | 2 |
| Human | BG772360.1 | May 15, 2001 | 3 |
| Human | BM797107.1 | Mar. 5, 2002 | 4 |
| Human | NM_032951.1 | Jul. 5, 2001* | 5 |
| Human | NM_032952.1 | Jul. 5, 2001* | 6 |
| Human | NM_032953.1 | Jul. 5, 2001* | 7 |
| Human | NM_032954.1 | Jul. 5, 2001* | |
| Human | NM_032994.1 | Jul. 5, 2001*,** | 9 |
| Human | the complement of nucleotides | Apr. 11, 2003* | 10 |
| | 11040374 to 11072520 of | | |
| | NT_007758.10 | | |
| Mouse | AF156604.1 | May 8, 2000 | |
| Mouse | AF245475.1 | Mar. 20, 2001 | 12 |
| Mouse | AF245476.1 | Mar. 20, 2001 | 13 |
| Mouse | AF245477.1 | Mar. 20, 2001 | |
| Mouse | AF245478.1 | Mar. 20, 2001 | 15 |
| Mouse | AF245479.1 | Mar. 20, 2001 | 16 |
| Mouse | BE647801.1 | Sep. 6, 2000 | 17 |
| Mouse | NM_021455.1 | Oct. 23, 2000* | |
| Mouse | nucleotides 837000 to 869000 | Oct. 9, 2001 | 18 |
| | of NT_029829.1 | | |

*updated and/or earlier versions available at GenBank
**sequence permanently suppressed because it is a nonsense-mediated mRNA decay

### (NMD) Candidate

The locations on the target nucleic acid to which active oligomeric compounds hybridize are hereinbelow referred to as “active target segments.” As used herein the term “active target segment” is defined as a portion of a target nucleic acid to which an at least one active antisense compound hybridizes and reduces expression of the target nucleic acid. While not wishing to be bound by theory, these target segments represent portions of the target nucleic acid which are accessible for hybridization.

Embodiments of the present invention include oligomeric compounds comprising sequences of 13 to 30 nucleotides in length and at least two modifications selected from a modified internucleoside linkage, a modified nucleobase, or a modified sugar. In one embodiment, the oligomeric compounds of the present invention are chimeric oligonucleotides. In one embodiment, the oligomeric compounds of the present invention are chimeric oligonucleotides comprising a deoxy nucleotide region flanked on each of the 5′ and 3′ ends with at least one 2′-O-(2-methoxyethyl) nucleotide. In another embodiment, the oligomeric compounds of the present invention are chimeric oligonucleotides comprising ten deoxynucleotides and flanked on both the 5′ and 3′ ends with five 2′-O-(2-methoxyethyl) nucleotides. In another embodiment, the oligomeric compounds of the present invention are chimeric oligonucleotides comprising fourteen deoxynucleotides and flanked on both the 5′ and 3′ ends with three 2′-O-(2-methoxyethyl) nucleotides. In another embodiment, the oligomeric compounds of the present invention are chimeric oligonucleotides comprising sixteen deoxynucleotides and flanked on both the 5′ and 3′ ends with two 2′-O-(2-methoxyethyl) nucleotides. In a further embodiment, the oligomeric compounds of the present invention may have at least one 5-methylcytosine.

In one embodiment the oligomeric compounds hybridize with ChREBP. In another embodiment, the oligomeric compounds inhibit the expression of ChREBP. In other embodiments, the oligomeric compounds inhibit the expression of ChREBP wherein the expression of ChREBP is inhibited by at least 40%, by at least 50%, by at least 60%, by at least 70%, by at least 80%, by at least 90%, or by 100%. One ordinarily skilled in the art will fully understand that the percentage inhibition of a target nucleic acid by an oligomeric compound will vary from assay-to-assay.

In one embodiment, the oligomeric compounds inhibit expression of ChREBP in cells, tissues or animals.

In one embodiment, the present invention provides methods of lowering plasma triglyceride levels in an animal by administering an oligomeric compound which inhibits ChREBP expression. In another embodiment, the present invention provides methods of lowering plasma glucose in an animal by administering an oligomeric compound which inhibits ChREBP expression. In another embodiment, the present invention provides methods of improving insulin sensitivity in an animal by administering an oligomeric compound which inhibits ChREBP expression. In another embodiment, improvement in insulin sensitivity is indicated by a reduction in circulating insulin levels.

Other embodiments of the invention include preventing, ameliorating or lessening the severity of a disease or condition in an animal by administering an oligomeric compound which inhibits ChREBP expression. Diseases or conditions include, but are not limited to, metabolic and cardiovascular disorders. Metabolic disorders include, but are not limited to, obesity, diet-induced obesity, diabetes, insulin resistance, insulin deficiency, dyslipidemia, hyperlipidemia, hypercholesterolemia, hyperglycemia, hypertriglyceridemia, hyperfattyacidemia, liver steatosis and metabolic syndrome. Cardiovascular disorders include, but are not limited to, coronary heart disease. Also provided are methods of improving cardiovascular risk profile in an animal by improving one or more cardiovascular risk factors by administering an oligomeric compound of the invention.

It is well known by those skilled in the art that it is possible to increase or decrease the length of an antisense compound and/or introduce mismatch bases without eliminating activity. For example, in Woolf et al. (Proc. Natl. Acad. Sci. USA 89:7305-7309, 1992, incorporated herein by reference), a series of ASOs 13-25 nucleobases in length were tested for their ability to induce cleavage of a target RNA in an oocyte injection model. ASOs 25 nucleobases in length with 8 or 11 mismatch bases near the ends of the ASOs were able to direct specific cleavage of the target mRNA, albeit to a lesser extent than the ASOs that contained no mismatches. Similarly, target specific cleavage was achieved using a 13 nucleobase ASOs, including those with 1 or 3 mismatches. Maher and Dolnick (Nuc. Acid. Res. 16:3341-3358, 1988, incorporated herein by reference) tested a series of tandem 14 nucleobase ASOs, and a 28 and 42 nucleobase ASOs comprised of the sequence of two or three of the tandem ASOs, respectively, for their ability to arrest translation of human DHFR in a rabbit reticulocyte assay. Each of the three 14 nucleobase ASOs alone were able to inhibit translation, albeit at a more modest level than the 28 or 42 nucleobase ASOs.

The oligomeric compounds in accordance with this invention may comprise a complementary oligomeric compound from about 13 to about 80 nucleobases (i.e. from about 13 to about 80 linked nucleosides). In other words, a single-stranded compound of the invention comprises from 13 to about 80 nucleobases, and a double-stranded antisense compound of the invention (such as a siRNA, for example) comprises two strands, each of which is from about 13 to about 80 nucleobases. Contained within the oligomeric compounds of the invention (whether single or double stranded and on at least one strand) are antisense portions. The “antisense portion” is that part of the oligomeric compound that is designed to work by an antisense mechanism. One of ordinary skill in the art will appreciate that this comprehends antisense portions of 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the oligomeric compounds of the invention have antisense portions of 13 to 50 nucleobases. One having ordinary skill in the art will appreciate that this embodies oligomeric compounds having antisense portions of 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the oligomeric compounds of the invention have antisense portions of 13 to 30 nucleobases. One having ordinary skill in the art will appreciate that this embodies oligomeric compounds having antisense portions of 13, 14, 15, 16, 17, 18, 19, 20, 21, 22, 23, 24, 25, 26, 27, 28, 29 or 30 nucleobases.

In some embodiments, the oligomeric compounds of the invention have antisense portions of 13 to 24 nucleobases. One having ordinary skill in the art will appreciate that this embodies oligomeric compounds having antisense portions of 13, 14, 15, 16, 17, 18, 19, 20, 21, 22, 23 or 24 nucleobases.

In one embodiment, the oligomeric compounds of the invention have antisense portions of 19 to 23 nucleobases. One having ordinary skill in the art will appreciate that this embodies oligomeric compounds having antisense portions of 19, 20, 21, 22 or 23 nucleobases.

In one embodiment, the oligomeric compounds of the invention have antisense portions of 20 to 80 nucleobases. One having ordinary skill in the art will appreciate that this embodies oligomeric compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the oligomeric compounds of the invention have antisense portions of 20 to 50 nucleobases. One having ordinary skill in the art will appreciate that this embodies oligomeric compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 20 to 30 nucleobases. One having ordinary skill in the art will appreciate that this embodies oligomeric compounds having antisense portions of 20, 21, 22, 23, 24, 25, 26, 27, 28, 29, or 30 nucleobases.

In one embodiment, the antisense compounds of the invention have antisense portions of 20 to 24 nucleobases. One having ordinary skill in the art will appreciate that this embodies oligomeric compounds having antisense portions of 20, 21, 22, 23, or 24 nucleobases.

In one embodiment, the oligomeric compounds of the invention have antisense portions of 20 nucleobases.

In one embodiment, the oligomeric compounds of the invention have antisense portions of 19 nucleobases.

In one embodiment, the oligomeric compounds of the invention have antisense portions of 18 nucleobases.

In one embodiment, the oligomeric compounds of the invention have antisense portions of 17 nucleobases.

In one embodiment, the oligomeric compounds of the invention have antisense portions of 16 nucleobases.

In one embodiment, the oligomeric compounds of the invention have antisense portions of 15 nucleobases.

In one embodiment, the oligomeric compounds of the invention have antisense portions of 14 nucleobases.

In one embodiment, the oligomeric compounds of the invention have antisense portions of 13 nucleobases.

Oligomeric compounds 13-80 nucleobases in length comprising a stretch of at least thirteen (13) consecutive nucleobases selected from within the illustrative antisense compounds are considered to be suitable antisense compounds as well.

Compounds of the invention include oligonucleotide sequences that comprise at least the thirteen consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about thirteen to about 80 nucleobases). Other compounds are represented by oligonucleotide sequences that comprise at least the 13 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about thirteen to about 80 nucleobases). It is also understood that compounds may be represented by oligonucleotide sequences that comprise at least thirteen consecutive nucleobases from an internal portion of the sequence of an illustrative compound, and may extend in either or both directions until the oligonucleotide contains about 13 to about 80 nucleobases.

One having skill in the art armed with the antisense compounds illustrated herein will be able, without undue experimentation, to identify further antisense compounds.

### Phenotypic Assays

Once modulator compounds of ChREBP have been identified by the methods disclosed herein, the compounds can be further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of ChREBP in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Measurement of the expression of one or more of the genes of the cell after treatment is also used as an indicator of the efficacy or potency of the ChREBP modulators. Hallmark genes, or those genes suspected to be associated with a specific disease state, condition, or phenotype, are measured in both treated and untreated cells.

### Kits, Research Reagents, Diagnostics, and Therapeutics

The oligomeric compounds of the present invention can be utilized for diagnostics, therapeutics, prophylaxis and as research reagents and kits. Furthermore, antisense compounds, which are able to inhibit gene expression with specificity, are often used by those of ordinary skill to elucidate the function of particular genes or to distinguish between functions of various members of a biological pathway.

For use in kits and diagnostics, the oligomeric compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more compounds or compositions of the present invention are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al, J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

Compounds of the invention can be used to modulate the expression of ChREBP in an animal, such as a human. In one non-limiting embodiment, the methods comprise the step of administering to said animal an effective amount of an antisense compound that inhibits expression of ChREBP. In one embodiment, the antisense compounds of the present invention effectively inhibit the levels or function of ChREBP RNA. Because reduction in ChREBP mRNA levels can lead to alteration in ChREBP protein products of expression as well, such resultant alterations can also be measured. Antisense compounds of the present invention that effectively inhibit the levels or function of an ChREBP RNA or protein products of expression is considered an active antisense compound. One ordinarily skilled in the art will know that the percent inhibition of an RNA encoding CHREBP will vary from assay to assay due to assay conditions and so the empirical data reported in any assay will not be exactly the same as a subsequent assay. In one embodiment, the antisense compounds of the invention inhibit the expression of ChREBP causing a reduction of the RNA encoding CHREBP by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of ChREBP can be measured in a bodily fluid, tissue or organ of the animal. Bodily fluids include, but are not limited to, blood (serum or plasma), lymphatic fluid, cerebrospinal fluid, semen, urine, synovial fluid and saliva and can be obtained by methods routine to those skilled in the art. Tissues or organs include, but are not limited to, blood (e.g., hematopoietic cells, such as human hematopoietic progenitor cells, human hematopoietic stem cells, CD34+ cells CD4+ cells), lymphocytes and other blood lineage cells, skin, bone marrow, spleen, thymus, lymph node, brain, spinal cord, heart, skeletal muscle, liver, pancreas, prostate, kidney, lung, oral mucosa, esophagus, stomach, ilium, small intestine, colon, bladder, cervix, ovary, testis, mammary gland, adrenal gland, and adipose (white and brown). Samples of tissues or organs can be routinely obtained by biopsy. In some alternative situations, samples of tissues or organs can be recovered from an animal after death.

The cells contained within said fluids, tissues or organs being analyzed can contain a nucleic acid molecule encoding ChREBP protein and/or the ChREBP-encoded protein itself. For example, fluids, tissues or organs procured from an animal can be evaluated for expression levels of the target mRNA or protein. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the target gene expression in the aforementioned fluids, tissues or organs, collected from an animal contacted with one or more compounds of the invention, by routine clinical methods known in the art. These biomarkers include but are not limited to: glucose levels, cholesterol levels, lipoprotein levels, triglyceride levels, free fatty acid levels and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

The compounds of the present invention can be utilized in pharmaceutical compositions by adding an effective amount of a compound to a suitable pharmaceutically acceptable diluent or carrier. In one aspect, the compounds of the present invention selectively inhibit the expression of ChREBP. The compounds of the invention can also be used in the manufacture of a medicament for the treatment of diseases and disorders related to ChREBP expression.

Methods whereby bodily fluids, organs or tissues are contacted with an effective amount of one or more of the antisense compounds or compositions of the invention are also contemplated. Bodily fluids, organs or tissues can be contacted with one or more of the compounds of the invention resulting in modulation of CHREBP expression in the cells of bodily fluids, organs or tissues. An effective amount can be determined by monitoring the modulatory effect of the antisense compound or compounds or compositions on target nucleic acids or their products or the effects on biomarkers of a disease or condition using methods routine to the skilled artisan. Further contemplated are ex vivo methods of treatment whereby cells or tissues are isolated from a subject, contacted with an effective amount of the antisense compound or compounds or compositions and reintroduced into the subject by routine methods known to those skilled in the art.

In one embodiment, provided are uses of a compound of an isolated double stranded RNA oligonucleotide in the manufacture of a medicament for inhibiting ChREBP expression or overexpression. Thus, provided herein is the use of an isolated double stranded RNA oligonucleotide targeted to ChREBP in the manufacture of a medicament for the treatment of a disease or disorder by means of the method described above.

### DEFINITIONS

“Antisense mechanisms” are all those involving hybridization of a compound with target nucleic acid, wherein the outcome or effect of the hybridization is either target degradation or target occupancy with concomitant stalling of the cellular machinery involving, for example, transcription or splicing.

### Targets

As used herein, the terms “target nucleic acid” and “nucleic acid molecule encoding CHREBP” have been used for convenience to encompass DNA encoding ChREBP, RNA (including pre-mRNA and mRNA or portions thereof) transcribed from such DNA, and also cDNA derived from such RNA.

### Regions, Segments, and Sites

The targeting process usually also includes determination of at least one target region, segment, or site within the target nucleic acid for the antisense interaction to occur such that the desired effect, e.g., modulation of expression, will result. “Region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as unique nucleobase positions within a target nucleic acid.

Once one or more target regions, segments or sites have been identified, oligomeric compounds are designed which are sufficiently complementary to the target, i.e., hybridize sufficiently well and with sufficient specificity, to give the desired effect.

Since, as is known in the art, the translation initiation codon is typically 5′ AUG (in transcribed mRNA molecules; 5′ ATG in the corresponding DNA molecule), the translation initiation codon is also referred to as the “AUG codon,” the “start codon” or the “AUG start codon.” A minority of genes have a translation initiation codon having the RNA sequence 5′ GUG, 5′ UUG or 5′ CUG, and 5′ AUA, 5′ ACG and 5′ CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. “Start codon” and “translation initiation codon” refer to the codon or codons that are used in vivo to initiate translation of an mRNA transcribed from a gene encoding a protein, regardless of the sequence(s) of such codons. It is also known in the art that a translation termination codon (or “stop codon”) of a gene may have one of three sequences, i.e., 5′ UAA, 5′ UAG and 5′ UGA (the corresponding DNA sequences are 5′ TAA, 5′ TAG and 5′ TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with oligomeric compounds of the invention.

The open reading frame (ORF) or “coding region,” which is known in the art to refer to the region between the translation initiation codon and the translation termination codon, is also a region which may be targeted effectively. Within the context of the present invention, one region is the intragenic region encompassing the translation initiation or termination codon of the open reading frame (ORF) of a gene.

Other target regions include the “5′untranslated region” (5′UTR, known in the art to refer to the portion of an mRNA in the 5′ direction from the translation initiation codon, and thus including nucleotides between the 5′ cap site and the translation initiation codon of an mRNA (or corresponding nucleotides on the gene), and the “3′ untranslated region” (3′UTR), known in the art to refer to the portion of an mRNA in the 3′ direction from the translation termination codon, and thus including nucleotides between the translation termination codon and 3′ end of an mRNA (or corresponding nucleotides on the gene). The “5′ cap site” of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. The 5′ cap region is also a target.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence, resulting in exon-exon junctions at the site where exons are joined. Targeting exon-exon junctions can be useful in situations where aberrant levels of a normal splice product are implicated in disease, or where aberrant levels of an aberrant splice product are implicated in disease. Targeting splice sites, i.e., intron-exon junctions or exon-intron junctions can also be particularly useful in situations where aberrant splicing is implicated in disease, or where an overproduction of a particular splice product is implicated in disease. Aberrant fusion junctions due to rearrangements or deletions are also suitable targets. mRNA transcripts produced via the process of splicing of two (or more) mRNAs from different gene sources are known as “fusion transcripts” and are also suitable targets. It is also known that introns can be effectively targeted using antisense compounds targeted to, for example, DNA or pre-mRNA. Single-stranded antisense compounds such as oligonucleotide compounds that work via an RNase H mechanism are effective for targeting pre-mRNA. Antisense compounds that function via an occupancy-based mechanism are effective for redirecting splicing as they do not, for example, elicit RNase H cleavage of the mRNA, but rather leave the mRNA intact and promote the yield of desired splice product(s).

### Variants

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Consequently, the types of variants described herein are also suitable target nucleic acids.

A search of the National Center for Biotechnology Information database revealed alternative mRNA variants of ChREBP which are the result of alternative splicing. Table 1 shows many of these variants. It is advantageous to selectively inhibit the expression of one or more variants of CHREBP.

### Active Target Segments

Active target segments are defined as being a segment of the target nucleic acid that is accessible to antisense hybridization and so is suitable for antisense modulation. The active target segments comprise at least two active antisense compounds that modulate the expression of the target nucleic acid. The at least two active antisense compounds are preferably separated on the target nucleic acid by about 60 nucleobases, more preferably by about 30 nucleobases, most preferably they are contiguous and most preferably they overlap. Those of ordinary skill in the art will readily appreciate that the this recited juxtaposition between the at least two active antisense compounds is not limited to these specific values, but rather includes any number of nucleobase separation within the spirit of this discussion.

The term “active antisense compound” is used herein to refer to an oligomeric compound that is determined to modulate the expression of a target nucleic acid.

The active target segments identified herein can be employed in a screen for additional compounds that modulate the expression of CHREBP. The screening method comprises the steps of contacting an active target segment of a nucleic acid molecule encoding ChREBP with one or more candidate modulators, typically an oligomeric compound, and selecting for one or more candidate modulators which perturb the expression of a nucleic acid molecule encoding ChREBP. Once it is shown that the candidate modulator or modulators are capable of modulating the expression of a nucleic acid molecule encoding ChREBP, the modulator can then be employed in further investigative studies of the function of ChREBP, or for use as a research, diagnostic, or therapeutic agent.

### Modulation of Target Expression

“Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. “Modulators” are those compounds that modulate the expression of ChREBP and which comprise at least a 13-nucleobase portion which is complementary to a active target segment.

Modulation of expression of a target nucleic acid can be achieved through alteration of any number of nucleic acid (DNA or RNA) functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of ChREBP. Thus, in one embodiment modulation of expression can mean an increase or decrease in target RNA or protein levels. In another embodiment modulation of expression can mean an increase or decrease of one or more RNA splice products, or a change in the ratio of two or more splice products.

### Hybridization and Complementarity

“Hybridization” means the pairing of complementary strands of oligomeric compounds. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances. An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refer to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

“Complementarity,” as used herein, refers to the capacity for precise pairing between two nucleobases on one or two oligomeric compound strands. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds of the present invention comprise at least 70%, or at least 75%, or at least 80%, or at least 85%, or at least 90%, or at least 92%, or at least 95%, or at least 97%, or at least 98%, or at least 99% sequence complementarity to a target region within the target nucleic acid sequence to which they are targeted. For example, an oligomeric compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Oligomeric Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound,” “antisense oligomeric compound” or “active antisense compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides, alternate splicers, and siRNAs. The oligomeric compounds may, optionally, comprise a second complementary strand (or may form a hairpin) in order to allow the compound to work through alternate antisense mechanisms (e.g., RNAi). As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

As used herein, the term “siRNA” is defined as a double-stranded compound having a first and second strand and comprises a central complementary portion between said first and second strands and terminal portions that are optionally complementary between said first and second strands or with the target mRNA. The ends of the strands may be modified by the addition of one or more natural or modified nucleobases to form an overhang.

As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang.

As used herein the term “blunt-ended siRNA” is defined as an siRNA having no terminal overhangs. That is, at least one end of the double-stranded compound is blunt.

“Chimeric” or “chimeras,” in the context of this invention, refers to oligomeric compounds, antisense compounds, antisense oligomeric compounds or active antisense compounds that can be single-or double-stranded oligomeric compounds, such as oligonucleotides, and which contain two or more chemically distinct regions, each comprising at least one monomer unit, i.e., a nucleotide in the case of an oligonucleotide compound.

A “gapmer” is defined as an oligomeric compound having a 2′-deoxyoligonucleotide region flanked by non-deoxyoligonucleotide segments. The central region is referred to as the “gap.” The flanking segments are referred to as “wings.” If one of the wings has zero non-deoxyoligonucleotide monomers, a “hemimer” is described.

### Chemical Modifications

### Modified Internucleoside Linkages

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Specific examples of oligomeric compounds of the present invention include oligonucleotides containing modified e.g. non-naturally occurring internucleoside linkages. Oligomeric compounds can have one or more modified internucleoside linkages. Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, phosphonoacetate and thiophosphonoacetate (see Sheehan et al., Nucleic Acids Research, 2003, 31(14), 4109-4118 and Dellinger et al., J. Am. Chem. Soc., 2003, 125, 940-950), selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage, i.e., a single inverted nucleoside residue which may be abasic (the nucleobase is missing or has a hydroxyl group in place thereof). Various salts, mixed salts and free acid forms are also included.

N3′-P5′-phosphoramidates have been reported to exhibit both a high affinity towards a complementary RNA strand and nuclease resistance (Gryaznov et al., J. Am. Chem. Soc., 1994, 116, 3143-3144). N3′-P5′-phosphoramidates have been studied with some success in vivo to specifically down regulate the expression of the c-myc gene (Skorski et al., Proc. Natl. Acad. Sci., 1997, 94, 3966-3971; and Faira et al., Nat. Biotechnol., 2001, 19, 40-44).

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In some embodiments of the invention, oligomeric compounds may have one or more phosphorothioate and/or hetero atom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Amide internucleoside linkages are disclosed in the above referenced U.S. Pat. No. 5,602,240.

Some oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

### Modified Sugars

Oligomeric compounds may also contain one or more substituted sugar moieties. Suitable compounds can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504), i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—(CH₂)₂—O—(CH₂)₂—N(CH₃)₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and, 6,147,200.

### DNA-Like and RNA-Like Conformations

The terms used to describe the conformational geometry of homoduplex nucleic acids are “A Form” for RNA and “B Form” for DNA. In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger et al., Principles of Nucleic Acid Structure, 1984, Springer-Verlag; New York, N.Y.). As used herein, B-form geometry is inclusive of both C2′-endo pucker and O4′-endo pucker.

The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). Consequently, compounds that favor an A-form geometry can enhance stacking interactions, thereby increasing the relative Tm and potentially enhancing a compound's antisense effect.

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA-like nucleosides so that particular properties of an oligomeric compound can be enhanced while maintaining the desirable 3′-endo conformational geometry.

There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include but are not limited to: modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. Also provided herein are oligomeric triggers of RNAi having one or more nucleosides modified in such a way as to favor a C3′-endo type conformation.

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2° F.-nucleosides (Kawasaki et al., J. Med. Chem. (1993), 36, 831-841), which adopts the 3′-endo conformation positioning the electronegative fluorine atom in the axial position. Representative 2′-substituent groups amenable to the present invention that give A-form conformational properties (3′-endo) to the resultant duplexes include 2′-O-alkyl, 2′-O-substituted alkyl and 2′-fluoro substituent groups. Other suitable substituent groups are various alkyl and aryl ethers and thioethers, amines and monoalkyl and dialkyl substituted amines.

Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA™, Morita et al, Bioorganic & Medicinal Chemistry Letters (2002), 12, 73-76.)

It is further intended that multiple modifications can be made to one or more of the oligomeric compounds of the invention at multiple sites of one or more monomeric subunits (nucleosides are suitable) and or internucleoside linkages to enhance properties such as but not limited to activity in a selected application.

The synthesis of numerous of the modified nucleosides amenable to the present invention are known in the art (see for example, Chemistry of Nucleosides and Nucleotides Vol 1-3, ed. Leroy B. Townsend, 1988, Plenum press). The conformation of modified nucleosides and their oligomers can be estimated by various methods routine to those skilled in the art such as molecular dynamics calculations, nuclear magnetic resonance spectroscopy and CD measurements.

### Oligonucleotide Mimetics

The term “mimetic” as it is applied to oligonucleotides includes oligomeric compounds wherein the furanose ring or the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid.

One such oligomeric compound, an oligonucleotide mimetic that has been shown to have excellent hybridization properties, is referred to as a peptide nucleic acid (PNA) (Nielsen et al., Science, 1991, 254, 1497-1500). PNAs have favorable hybridization properties, high biological stability and are electrostatically neutral molecules. PNA compounds have been used to correct aberrant splicing in a transgenic mouse model (Sazani et al., Nat. Biotechnol., 2002, 20, 1228-1233). In PNA oligomeric compounds, the sugar-backbone of an oligonucleotide is replaced with an amide containing backbone, in particular an aminoethylglycine backbone. The nucleobases are bound directly or indirectly to aza nitrogen atoms of the amide portion of the backbone. Representative United States patents that teach the preparation of PNA oligomeric compounds include, but are not limited to, U.S. Pat. Nos. 5,539,082; 5,714,331; and 5,719,262. PNA compounds can be obtained commercially from Applied Biosystems (Foster City, Calif., USA). Numerous modifications to the basic PNA backbone are known in the art; particularly useful are PNA compounds with one or more amino acids conjugated to one or both termini. For example, 1-8 lysine or arginine residues are useful when conjugated to the end of a PNA molecule.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups have been selected to give a non-ionic oligomeric compound. Morpholino-based oligomeric compounds are non-ionic mimetics of oligo-nucleotides which are less likely to form undesired interactions with cellular proteins (Dwaine A. Braasch and David R. Corey, Biochemistry, 2002, 41(14), 4503-4510). Morpholino-based oligomeric compounds have been studied in zebrafish embryos (see: Genesis, volume 30, issue 3, 2001 and Heasman, J., Dev. Biol., 2002, 243, 209-214). Further studies of morpholino-based oligomeric compounds have also been reported (Nasevicius et al., Nat. Genet., 2000, 26, 216-220; and Lacerra et al., Proc. Natl. Acad. Sci., 2000, 97, 9591-9596). Morpholino-based oligomeric compounds are disclosed in U.S. Pat. No. 5,034,506. The morpholino class of oligomeric compounds has been prepared having a variety of different linking groups joining the monomeric subunits. Linking groups can be varied from chiral to achiral, and from charged to neutral. U.S. Pat. No. 5,166,315 discloses linkages including —O—P(═O)(N(CH₃)₂)—O—; U.S. Pat. No. 5,034,506 discloses achiral intermorpholino linkages; and U.S. Pat. No. 5,185,444 discloses phosphorus containing chiral intermorpholino linkages.

A further class of oligonucleotide mimetic is referred to as cyclohexene nucleic acids (CeNA). In CeNA oligonucleotides, the furanose ring normally present in a DNA or RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. coli RNase H resulting in cleavage of the target RNA strand.

A further modification includes bicyclic sugar moieties such as “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. LNA's are commercially available from ProLigo (Paris, France and Boulder, Colo., USA).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research, 2003, 21, 6365-6372).

Another similar bicyclic sugar moiety that has been prepared and studied has the bridge going from the 3′-hydroxyl group via a single methylene group to the 4′ carbon atom of the sugar ring thereby forming a 3′-C,4′-C-oxymethylene linkage (see U.S. Pat. No. 6,043,060).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11° C.) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands. DNA LNA chimeras have been shown to efficiently inhibit gene expression when targeted to a variety of regions (5′-untranslated region, region of the start codon or coding region) within the luciferase mRNA (Braasch et al., Nucleic Acids Research, 2002, 30, 5160-5167).

Antisense oligonucleotides containing LNAs have been described (Wahlestedt et al., Proc. Natl. Acad. Sci. U.S.A., 2000, 97, 5633-5638). LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished. Further successful in vivo studies involving LNA's have shown knock-down of the rat delta opioid receptor without toxicity (Wahlestedt et al., Proc. Natl. Acad. Sci., 2000, 97, 5633-5638) and in another study showed a blockage of the translation of the large subunit of RNA polymerase II (Fluiter et al., Nucleic Acids Res., 2003, 31, 953-962).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Further oligonucleotide mimetics have been prepared to include bicyclic and tricyclic nucleoside analogs (see Steffens et al., Helv. Chim. Acta, 1997, 80, 2426-2439; Steffens et al., J. Am. Chem. Soc., 1999, 121, 3249-3255; Renneberg et al., J. Am. Chem. Soc., 2002, 124, 5993-6002; and Renneberg et al., Nucleic acids res., 2002, 30, 2751-2757). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids which incorporate a phosphorus group in the backbone. This class of oligonucleotide mimetic is reported to have useful physical and biological and pharmacological properties in the areas of inhibiting gene expression (antisense oligonucleotides, sense oligonucleotides and triplex-forming oligonucleotides), as probes for the detection of nucleic acids and as auxiliaries for use in molecular biology. Further oligonucleotide mimetics amenable to the present invention have been prepared wherein a cyclobutyl ring replaces the naturally occurring furanosyl ring.

### Modified and Alternate Nucleobases

The oligomeric compounds of the invention also include oligonucleotides in which a different base is present at one or more of the nucleotide positions in the compound. For example, if the first nucleotide is an adenosine, oligonucleotides may be produced which contain thymidine, guanosine or cytidine at this position. This may be done at any of the positions of the oligomeric compound. These compounds are then tested using the methods described herein to determine their ability to inhibit expression of ChREBP mRNA.

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in the art as suitable for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692.

Oligomeric compounds of the present invention can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Grant Publications 20030207804 and 20030175906).

Further helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° C. relative to 5-methyl cytosine (dC5^{me}, which is a high affinity enhancement for a single modification. On the other hand, the gain in helical stability does not compromise the specificity of the oligonucleotides.

Further tricyclic heterocyclic compounds and methods of using them that are amenable to use in the present invention are disclosed in U.S. Pat. Nos. 6,028,183, and 6,007,992.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNase H, enhance cellular uptake and exhibit an increased antisense activity (Lin, K-Y; Matteucci, M. J. Am. Chem. Soc. 1998, 120, 8531-8532). The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

Further modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Pre-Grant Publication 20030158403.

### Conjugates

Another modification of the oligomeric compounds of the invention involves chemically linking to the oligomeric compound one or more moieties or conjugates which enhance the properties of the oligomeric compound, such as to enhance the activity, cellular distribution or cellular uptake of the oligomeric compound. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. Nos. 6,287,860 and 6,762,169.

Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligomeric compounds of the invention may also be conjugated to drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. Pat. No. 6,656,730.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds can also be modified to have one or more stabilizing groups that are generally attached to one or both termini of an oligomeric compound to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270). These terminal modifications protect the oligomeric compounds having terminal nucleic acid molecules from exonuclease degradation, and can improve delivery and/or localization within a cell. The cap can be present at either the 5′-terminus (5′-cap) or at the 3′-terminus (3′-cap) or can be present on both termini of a single strand, or one or more termini of both strands of a double-stranded compound. This cap structure is not to be confused with the inverted methylguanosine “5′cap” present at the 5′ end of native mRNA molecules. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofaranosyl nucleotide; acyclic 3′,4′-seco nucleotide; acyclic 3,4-dihydroxybutyl nucleotide; acyclic 3,5-dihydroxypentyl ribonucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270). For siRNA constructs, the 5′ end (5′ cap) is commonly but not limited to 5′-hydroxyl or 5′-phosphate.

Particularly suitable 3′-cap structures include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Further 3′ and 5′-stabilizing groups that can be used to cap one or both ends of an oligomeric compound to impart nuclease stability include those disclosed in WO 03/004602 published on Jan. 16, 2003.

### Chimeric Compounds

It is not necessary for all positions in a given oligomeric compound to be uniformly modified, and in fact more than one of the aforementioned modifications may be incorporated in a single compound or even within a single nucleoside within an oligomeric compound.

The present invention also includes oligomeric compounds which are chimeric compounds. These oligonucleotides typically contain at least one region which is modified so as to confer upon the oligonucleotide increased resistance to nuclease degradation, increased cellular uptake, alteration of charge, increased stability and/or increased binding affinity for the target nucleic acid. An additional region of the oligonucleotide may serve as a substrate for RNAses or other enzymes. By way of example, RNAse H is a cellular endonuclease which cleaves the RNA strand of an RNA:DNA duplex. Activation of RNase H, therefore, results in cleavage of the RNA target when bound by a DNA-like oligomeric compound, thereby greatly enhancing the efficiency of oligonucleotide-mediated inhibition of gene expression. The cleavage of RNA:RNA hybrids can, in like fashion, be accomplished through the actions of endoribonucleases, such as RNase III or RNAseL which cleaves both cellular and viral RNA. Cleavage products of the RNA target can be routinely detected by gel electrophoresis and, if necessary, associated nucleic acid hybridization techniques known in the art.

Chimeric oligomeric compounds of the invention can be formed as composite structures of two or more oligonucleotides, modified oligonucleotides, oligonucleosides, oligonucleotide mimetics, or regions or portions thereof. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

An example of a chimeric oligonucleotide is a gapmer having a 2′-deoxyoligonucleotide region flanked by non-deoxyoligonucleotide segments. While not wishing to be bound by theory, the gap of the gapmer presents a substrate recognizable by RNase H when bound to the RNA target whereas the wings do not provide such a substrate but can confer other properties such as contributing to duplex stability or advantageous pharmacokinetic effects. Each wing can be one or more non-deoxyoligonucleotide monomers. In one embodiment, the gapmer is a ten deoxynucleotide gap flanked by five non-deoxynucleotide wings. This is referred to as a 5-10-5 gapmer. Other configurations are readily recognized by those skilled in the art. In one embodiment the wings comprise 2′-MOE modified nucleotides. In another embodiment the gapmer has a phosphorothioate backbone. In another embodiment the gapmer has 2′-MOE wings and a phosphorothioate backbone. Other suitable modifications are readily recognizable by those skilled in the art.

### NAFLD and Metabolic Syndrome

The term “nonalcoholic fatty liver disease” (NAFLD) encompasses a disease spectrum ranging from simple triglyceride accumulation in hepatocytes (hepatic steatosis) to hepatic steatosis with inflammation (steatohepatitis), fibrosis, and cirrhosis. Nonalcoholic steatohepatitis (NASH) occurs from progression of NAFLD beyond deposition of triglycerides. A second-hit capable of inducing necrosis, inflammation, and fibrosis is required for development of NASH. Candidates for the second-hit can be grouped into broad categories: factors causing an increase in oxidative stress and factors promoting expression of proinflammatory cytokines. It has been suggested that increased liver triglycerides lead to increased oxidative stress in hepatocytes of animals and humans, indicating a potential cause-and-effect relationship between hepatic triglyceride accumulation, oxidative stress, and the progression of hepatic steatosis to NASH (Browning and Horton, J. Clin. Invest., 2004, 114, 147-152). Hypertriglyceridemia and hyperfattyacidemia can cause triglyceride accumulation in peripheral tissues (Shimamura et al., Biochem. Biophys. Res. Commun., 2004, 322, 1080-1085).

“Metabolic syndrome” is defined as a clustering of lipid and non-lipid cardiovascular risk factors of metabolic origin. It is closely linked to the generalized metabolic disorder known as insulin resistance. The National Cholesterol Education Program (NCEP) Adult Treatment Panel III (ATPIII) established criteria for diagnosis of metabolic syndrome when three or more of five risk determinants are present. The five risk determinants are abdominal obesity defined as waist circumference of greater than 102 cm for men or greater than 88 cm for women, triglyceride levels greater than or equal to 150 mg/dL, HDL cholesterol levels of less than 40 mg/dL for men and less than 50 mg/dL for women, blood pressure greater than or equal to 130/85 mm Hg and fasting glucose levels greater than or equal to 110 mg/dL. These determinants can be readily measured in clinical practice (JAMA, 2001, 285, 2486-2497).

### HbAlc

HbAlc is a stable minor hemoglobin variant formed in vivo via posttranslational modification by glucose, and it contains predominantly glycated NH2-terminal β-chains. There is a strong correlation between levels of HbAlc and the average blood glucose levels over the previous 3 months. Thus HbAlc is often used for measuring sustained blood glucose control (Bunn, H. F. et al., 1978, Science. 200, 21-7). HbAlc can be measured by ion-exchange HPLC or immunoassay; home blood collection and mailing kits for HbAlc measurement are now widely available. Serum fructosamine is another measure of stable glucose control and can be measured by a calorimetric method (Cobas Integra, Roche Diagnostics).

### Cardiovascular Risk Profile

Conditions associated with risk of developing a cardiovascular disease include, but are not limited to, history of myocardial infarction, unstable angina, stable angina, coronary artery procedures (angioplasty or bypass surgery), evidence of clinically significant myocardial ischemia, noncoronary forms of atherosclerotic disease (peripheral arterial disease, abdominal aortic aneurysm, carotid artery disease), diabetes, cigarette smoking, hypertension, low HDL cholesterol, family history of premature CHD, obesity, physical inactivity, elevated triglyceride, or metabolic syndrome (Jama, 2001, 285, 2486-2497; Grundy et al., Circulation, 2004, 110, 227-239).

### Salts, Prodrugs and Bioequivalents

The oligomeric compounds of the present invention comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the oligomeric compounds of the present invention, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides of the invention are prepared as SATE ((S-acetyl-2-thioethyl) phosphate) derivatives according to the methods disclosed in WO 93/24510 or WO 94/26764. An additional prodrug of an antisense modulator of a target nucleic acid can mean an antisense compound that is cleaved in vivo to release the active and shorter antisense compound.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 22 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts of antisense oligonucleotides are useful and are well accepted for therapeutic administration to humans. In another embodiment, sodium salts of dsRNA compounds are also provided.

### Formulations

The oligomeric compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including but not limited to ophthalmic and to mucous membranes including vaginal and rectal delivery), pulmonary, e.g., by inhalation or insufflation of powders or aerosols, including by nebulizer (intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Sites of administration are known to those skilled in the art. Oligonucleotides with at least one 2′-O-methoxyethyl modification are believed to be useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Formulations for topical administration include those in which the oligomeric compounds of the invention are in admixture with a topical delivery agent such as lipids, liposomes, fatty acids, fatty acid esters, steroids, chelating agents and surfactants.

For topical or other administration, oligomeric compounds of the invention may be encapsulated within liposomes or may form complexes thereto, such as to cationic liposomes. Alternatively, oligonucleotides may be complexed to lipids, in particular to cationic lipids. Fatty acids and esters, pharmaceutically acceptable salts thereof, and their uses are further described in U.S. Pat. No. 6,287,860. Topical formulations are described in detail in U.S. patent application Ser. No. 09/315,298 filed on May 20, 1999.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

The pharmaceutical formulations and compositions of the present invention may also include surfactants. The use of surfactants in drug products, formulations and in emulsions is well known in the art. Surfactants and their uses are further described in U.S. Pat. No. 6,287,860.

In one embodiment, the present invention employs various penetration enhancers to affect the efficient delivery of oligomeric compounds, particularly oligonucleotides. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860.

In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property.

Oral compositions for administration of non-parenteral oligomeric compounds can be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. Such oral oligomeric compound compositions can be referred to as “mucosal penetration enhancers.”

Oligomeric compounds, such as oligonucleotides, may be delivered orally, in granular form including sprayed dried particles, or complexed to form micro or nanoparticles. Oligonucleotide complexing agents and their uses are further described in U.S. Pat. No. 6,287,860. Oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002.

In one embodiment, oral oligomeric compound compositions comprise at least one member of the group consisting of surfactants, fatty acids, bile salts, chelating agents, and non-chelating surfactants. Further embodiments comprise oral oligomeric compound comprising at least one fatty acid, e.g. capric or lauric acid, or combinations or salts thereof. One combination is the sodium salt of lauric acid, capric acid and UDCA.

In one embodiment, oligomeric compound compositions for oral delivery comprise at least two discrete phases, which phases may comprise particles, capsules, gel-capsules, microspheres, etc. Each phase may contain one or more oligomeric compounds, penetration enhancers, surfactants, bioadhesives, effervescent agents, or other adjuvant, excipient or diluent

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

Oral oligomeric compositions may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers.

One of skill in the art will recognize that formulations are routinely designed according to their intended use, i.e. route of administration.

### Combinations

Compositions of the invention can contain two or more oligomeric compounds. In another related embodiment, compositions of the present invention can contain one or more antisense compounds, particularly oligonucleotides, targeted to a first nucleic acid and one or more additional antisense compounds targeted to a second nucleic acid target. Alternatively, compositions of the present invention can contain two or more antisense compounds targeted to different regions of the same nucleic acid target. Two or more combined compounds may be used together or sequentially.

### Combination Therapy

The compounds of the invention may be used in combination therapies, wherein an additive effect is achieved by administering one or more compounds of the invention and one or more other suitable therapeutic/prophylactic compounds to treat a disease or a condition. Suitable therapeutic/prophylactic compound(s) include, but are not limited to, glucose-lowering agents, anti-obesity agents, and lipid lowering agents. Glucose lowering agents include, but are not limited to hormones or hormone mimetics (e.g., insulin, GLP-1 or a GLP-1 analog, exendin-4 or liraglutide), a sulfonylurea (e.g., acetohexamide, chlorpropamide, tolbutamide, tolazamide, glimepiride, a glipizide, glyburide or a gliclazide), a biguanide (metformin), a meglitinide (e.g., nateglinide or repaglinide), a thiazolidinedione or other PPAR-gamma agonists (e.g., pioglitazone or rosiglitazone), an alpha-glucosidase inhibitor (e.g., acarbose or miglitol), or an antisense compound not targeted to LMW-PTPase. Also included are dual PPAR-agonists (e.g., muraglitazar). Also included are diabetes treatments in development (e.g. LAF237, being developed by Novartis; MK-0431, being developed by Merck; or rimonabant, being developed by Sanofi-Aventis). Anti-obesity agents include, but are not limited to, appetite suppressants (e.g. phentermine or Meridia™), fat absorption inhibitors such as orlistat (e.g. Xenical™), and modified forms of ciliary neurotrophic factor which inhibit hunger signals that stimulate appetite. Lipid lowering agents include, but are not limited to, bile salt sequestering resins (e.g., cholestyramine, colestipol, and colesevelam hydrochloride), HMGCoA-reductase inhibitors (e.g., lovastatin, cerivastatin, prevastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), CETP inhibitors (e.g. torcetrapib and JTT-705) MTP inhibitors (eg, implitapide), inhibitors of bile acid transporters (apical sodium-dependent bile acid transporters), regulators of hepatic CYP7a, ACAT inhibitors (e.g. Avasimibe), estrogen replacement therapeutics (e.g., tamoxigen), synthetic HDL (e.g. ETC-216), anti-inflammatories (e.g., glucocorticoids), or an antisense compound not targeted to LMW-PTPase. One or more of these drugs may be combined with one or more of the antisense inhibitors of LMW-PTPase to achieve an additive therapeutic effect.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds of the present invention can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

### Precursor Compounds

The following precursor compounds, including amidites and their intermediates can be prepared by methods routine to those skilled in the art; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N⁴-benzoyl-5-methylcytidine penultimate intermediate for 5-methyl dC amidite, (5′-O-(4,4′-Dimethoxytriphenylmethyl)-2′-deoxy-N⁴-benzoyl-5-methylcytidin-3′-O-yl)-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, (5′-O-(4,4′-Dimethoxytriphenylmethyl)-2′-O-(2-methoxyethyl)-5-methyluridin-3′-O-yl)-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, (5′-O-(4,4′-Dimethoxytriphenylmethyl)-2′-O-(2-methoxyethyl)-N⁴-benzoyl-5-methylcytidin-3′-O-yl)-2-cyanoethyl-N,N-diisopropylphosphoramidite (MOE 5-Me-C amidite), (5′-O-(4,4′-Dimethoxytriphenylmethyl)-2′-O-(2-methoxyethyl)-N-6-benzoyladenosin-3′-O-yl)-2-cyanoethyl-N,N-diisopropylphosphoramidite (MOE A amdite), (5′-O-(4,4′-Dimethoxytriphenylmethyl)-2′-O-(2-methoxyethyl)-N⁴-isobutyrylguanosin-3′-O-yl)-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-((2-phthalimidoxy)ethyl)-5′-t-butyldiphenylsilyl-5-methyluridine, 5′-O-tert-butyldiphenylsilyl-2′-O-((2-formadoximinooxy)ethyl)-5-methyluridine, 5′-O-tert-Butyldiphenylsilyl-2′-O—(N,N dimethylaminooxyethyl)-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-((2-cyanoethyl)-N,N-diisopropylphosphoramidite), 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-((2-cyanoethyl)-N,N-diisopropylphosphoramidite), 2′-dimethylaminoethoxyethoxy (2′-DMAEOE) nucleoside amidites, 2′-O-(2(2-N,N-dimethylaminoethoxy)ethyl)-5-methyl uridine, 5′-O-dimethoxytrityl-2′-O-(2(2-N,N-dimethylaminoethoxy)-ethyl))-5-methyl uridine and 5′-O-Dimethoxytrityl-2′-O-(2(2-N,N-dimethylaminoethoxy)-ethyl))-5-methyl uridine-3′-O-(cyanoethyl-N,N-diisopropyl)phosphoramidite.

The preparation of these and other such precursor compounds for oligonucleotide synthesis are routine in the art and disclosed in U.S. Pat. No. 6,426,220 and published PCT WO 02/36743.

2′-Deoxy and 2′-methoxy beta-cyanoethyldiisopropyl phosphoramidites can be purchased from commercial sources (e.g. Chemgenes, Needham, Mass. or Glen Research, Inc. Sterling, Va.). Other 2′-O-alkoxy substituted nucleoside amidites can be prepared as described in U.S. Pat. No. 5,506,351.

Oligonucleotides containing 5-methyl-2′-deoxycytidine (5-Me—C) nucleotides can be synthesized routinely according to published methods (Sanghvi, et. al., Nucleic Acids Research, 1993, 21, 3197-3203) using commercially available phosphoramidites (Glen Research, Sterling Va. or ChemGenes, Needham, Mass.).

2′-fluoro oligonucleotides can be synthesized routinely as described (Kawasaki, et. al., J. Med. Chem., 1993, 36, 831-841) and U.S. Pat. No. 5,670,633.

2′-O-Methoxyethyl-substituted nucleoside amidites can be prepared routinely as per the methods of Martin, P., Helvetica Chimica Acta, 1995, 78, 486-504.

Aminooxyethyl and dimethylaminooxyethyl amidites can be prepared routinely as per the methods of U.S. Pat. No. 6,127,533.

### Oligonucleotide Synthesis

Phosphorothioate-containing oligonucleotides (P═S) can be synthesized by methods routine to those skilled in the art (see, for example, Protocols for Oligonucleotides and Analogs, Ed. Agrawal (1993), Humana Press). Phosphinate oligonucleotides can be prepared as described in U.S. Pat. No. 5,508,270.

Alkyl phosphonate oligonucleotides can be prepared as described in U.S. Pat. No. 4,469,863.

3′-Deoxy-3′-methylene phosphonate oligonucleotides can be prepared as described in U.S. Pat. No. 5,610,289 or 5,625,050.

Phosphoramidite oligonucleotides can be prepared as described in U.S. Pat. No. 5,256,775 or U.S. Pat. No. 5,366,878.

Alkylphosphonothioate oligonucleotides can be prepared as described in published PCT applications PCT/US94/00902 and PCT/US93/06976 (published as WO 94/17093 and WO 94/02499, respectively).

3′-Deoxy-3′-amino phosphoramidate oligonucleotides can be prepared as described in U.S. Pat. No. 5,476,925.

Phosphotriester oligonucleotides can be prepared as described in U.S. Pat. No. 5,023,243.

Borano phosphate oligonucleotides can be prepared as described in U.S. Pat. Nos. 5,130,302 and 5,177,198.

4′-thio-containing oligonucleotides can be synthesized as described in U.S. Pat. No. 5,639,873.

### Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages can be prepared as described in U.S. Pat. Nos. 5,378,825, 5,386,023, 5,489,677, 5,602,240 and 5,610,289.

Formacetal and thioformacetal linked oligonucleosides can be prepared as described in U.S. Pat. Nos. 5,264,562 and 5,264,564.

Ethylene oxide linked oligonucleosides can be prepared as described in U.S. Pat. No. 5,223,618.

### Peptide Nucleic Acid Synthesis

Peptide nucleic acids (PNAs) can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, 5,719,262, 6,559,279 and 6,762,281.

### Synthesis of 2′-O-Protected Oligomers/RNA Synthesis

Oligomeric compounds incorporating at least one 2′-O-protected nucleoside by methods routine in the art. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound.

A large number of 2′-O-protecting groups have been used for the synthesis of oligoribo-nucleotides and any can be used. Some of the protecting groups used initially for oligoribonucleotide synthesis included tetrahydropyran-1-yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese et al. have identified a number of piperidine derivatives (like Fpmp) that are useful in the synthesis of oligoribonucleotides including 1-[(chloro-4-methyl)phenyl]-4′-methoxypiperidin-4-yl (Reese et al., Tetrahedron Lett., 1986, (27), 2291). Another approach is to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group, initially used for the synthesis of oligoribonucleotides, is the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal. For example, the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the [2-(nitrobenzyl)oxy]methyl (nbm) protecting group (Schwartz et al., Bioorg. Med. Chem. Lett., 1992,

(2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O-[(triisopropylsilyl)oxy]methyl (2′-O—CH₂—O—Si(iPr)₃, TOM). One 2′-O-protecting group that was prepared to be used orthogonally to the TOM group was 2′-O—[(R)-1-(2-nitrophenyl)ethyloxy)methyl] ((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

The main RNA synthesis strategies that are presently being used commercially include 5′-O-DMT-2′-O-t-butyldimethylsilyl (TBDMS), 5′-O-DMT-2′-O-[1 (2-fluorophenyl)-4-methoxypiperidin-4-yl] (FPMP), 2′-O-[(triisopropylsilyl)oxy]methyl (2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). Some companies currently offering RNA products include Pierce Nucleic Acid Technologies (Milwaukee, Wis.), Dharmacon Research Inc. (a subsidiary of Fisher Scientific, Lafayette, Colo.), and Integrated DNA Technologies, Inc. (Coralville, Iowa). One company, Princeton Separations, markets an RNA synthesis activator advertised to reduce coupling times especially with TOM and TBDMS chemistries. Such an activator would also be amenable to the oligomeric compounds of the present invention.

All of the aforementioned RNA synthesis strategies are amenable to the oligomeric compounds of the present invention. Strategies that would be a hybrid of the above e.g. using a 5′-protecting group from one strategy with a 2′-O-protecting from another strategy is also contemplated herein.

### Synthesis of Chimeric Oligomeric Compounds

### (2′-O-Me)-(2′-deoxy)-(2′-O-Me) Chimeric Phosphorothioate Oligonucleotides

Chimeric oligonucleotides having 2′-o-alkyl phosphorothioate and 2′-deoxy phosphorothioate oligonucleotide segments can be routinely synthesized by one skilled in the art, using, for example, an Applied Biosystems automated DNA synthesizer Model 394. Oligonucleotides can be synthesized using an automated synthesizer and 2′-deoxy-5′-dimethoxytrityl-3′-O-phosphoramidite for the DNA portion and 5′-dimethoxytrityl-2′-O-methyl-3′-O-phosphoramidite for the 2′-O-alkyl portion. In one nonlimiting example, the standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligonucleotide is then recovered by an appropriate method (precipitation, column chromatography, volume reduced in vacuo) and analyzed by methods routine in the art.

### (2′-O-(2-Methoxyethyl))-(2′-deoxy)-(2′-O-(2-Methoxyethyl)) Chimeric Phosphorothioate Oligonucleotides

(2′-O-(2-methoxyethyl))-(2′-deoxy)-(−2′-O-(2-methoxyethyl)) chimeric phosphorothioate oligonucleotides can be prepared as per the procedure above for the 2′-O-methyl chimeric oligonucleotide, with the substitution of 2′-O-(methoxyethyl) amidites for the 2′-O-methyl amidites.

### (2′-O-(2-Methoxyethyl)Phosphodiester)-(2′-deoxy Phosphorothioate)-(2′-O-(2-Methoxyethyl) Phosphodiester) Chimeric Oligonucleotides

(2′-O-(2-methoxyethyl phosphodiester)-(2′-deoxy phosphorothioate)-(2′-O-(methoxyethyl) phosphodiester) chimeric oligonucleotides can be prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides can be synthesized according to U.S. Pat. No. 5,623,065.

### Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates.

### Nonlimiting Disclosure and Incorporation by Reference

While certain compounds, compositions and methods of the present invention have been described with specificity in accordance with certain embodiments, the examples herein serve only to illustrate the compounds of the invention and are not intended to limit the same. Each of the references, GENBANK® accession numbers, and the like recited in the present application is incorporated herein by reference in its entirety.

### Example 1

### Assaying Modulation of Expression

Modulation of ChREBP expression can be assayed in a variety of ways known in the art. ChREBP mRNA levels can be quantitated by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Levels of proteins encoded by ChREBP can be quantitated in a variety of ways well known in the art, such as immunoprecipitation, Western blot analysis (immunoblotting), ELISA or fluorescence-activated cell sorting (FACS). Antibodies directed to a protein encoded by ChREBP can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

The effect of oligomeric compounds of the present invention on target nucleic acid expression can be tested in any of a variety of cell types provided that the target nucleic acid is present at measurable levels. The effect of oligomeric compounds of the present invention on target nucleic acid expression can be routinely determined using, for example, PCR or Northern blot analysis. Cell lines are derived from both normal tissues and cell types and from cells associated with various disorders (e.g. hyperproliferative disorders). Cell lines derived from multiple tissues and species can be obtained from American Type Culture Collection (ATCC, Manassas, Va.). Additional cell lines, such as HuH-7 and U373, can be obtained from the Japanese Cancer Research Resources Bank (Tokyo, Japan) and the Centre for Applied Microbiology and Research (Wiltshire, United Kingdom), respectively.

Primary cells, or those cells which are isolated from an animal and not subjected to continuous culture, can be prepared according to methods known in the art or obtained from various commercial suppliers. Additionally, primary cells include those obtained from donor human subjects in a clinical setting (i.e. blood donors, surgical patients). Primary cells are prepared by methods known in the art or can be obtained from commercial suppliers such as StemCell Technologies (Seattle, Wash.); Zen-Bio, Inc. (Research Triangle Park, N.C.); Cambrex Biosciences (Walkersville, Md.); In Vitro Technologies (Baltimore, Md.); Cascade Biologics (Portland, Oreg.); Advanced Biotechnologies (Columbia, Md.).

### Cell Types

The effect of oligomeric compounds on target nucleic acid expression was tested in one or more of the following cell types.

### HepG2 Cells:

The human hepatoblastoma cell line HepG2 was obtained from the American Type Culture Collection (Manassas, Va.). HepG2 cells were routinely cultured in Eagle's MEM supplemented with 10% fetal bovine serum, 1 mM non-essential amino acids, and 1 mM sodium pyruvate (Invitrogen Life Technologies, Carlsbad, Calif.). Cells were routinely passaged by trypsinization and dilution when they reached approximately 90% confluence. Multiwell culture plates were prepared for cell culture by coating with a 1:100 dilution of type 1 rat tail collagen (BD Biosciences, Bedford, Mass.) in phosphate-buffered saline. The collagen-containing plates were incubated at 37° C. for approximately 1 hour, after which the collagen was removed and the wells were washed twice with phosphate-buffered saline. Cells were seeded into 96-well plates (Falcon-Primaria #3872) at a density of approximately 8,000 cells/well for use in oligomeric compound transfection experiments.

### 3T3-L1 Cells:

The mouse embryonic adipocyte-like cell line 3T3-L1 was obtained from the American Type Culture Collection (Manassas, Va.). 3T3-L1 cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 80% confluence. Cells were seeded into 96-well plates (Falcon-Primaria #3872) at a density of approximately 4000 cells/well for use in oligomeric compound transfection experiments.

Treatment with Oligomeric Compounds

When cells reach appropriate confluency, they were treated with oligonucleotide using a transfection method as described. Other suitable transfection reagents known in the art include, but are not limited to, LIPOFECTAMINE™, OLIGOFECTAMINE™, and FUGENE™. Other suitable transfection methods known in the art include, but are not limited to, electroporation.

### LIPOFECTIN™

When cells reached 65-75% confluency, they were treated with oligonucleotide. Oligonucleotide was mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

### Control Oligonucleotides

Control oligonucleotides are used to determine the optimal oligomeric compound concentration for a particular cell line. Furthermore, when oligomeric compounds of the invention are tested in oligomeric compound screening experiments or phenotypic assays, control oligonucleotides are tested in parallel with compounds of the invention. In some embodiments, the control oligonucleotides are used as negative control oligonucleotides, i.e., as a means for measuring the absence of an effect on gene expression or phenotype. In alternative embodiments, control oligonucleotides are used as positive control oligonucleotides, i.e., as oligonucleotides known to affect gene expression or phenotype. Control oligonucleotides are shown in Table 2. “Target Name” indicates the gene to which the oligonucleotide is targeted. “Species of Target” indicates species in which the oligonucleotide is perfectly complementary to the target mRNA. “Motif” is indicative of chemically distinct regions comprising the oligonucleotide. Certain compounds in Table 2 are composed of 2′-O-(2-methoxyethyl) nucleotides, also known as 2′-MOE nucleotides, and are designated as “Uniform MOE”. Compounds in Table 2 are chimeric oligonucleotides, composed of a central “gap” region consisting of 2′-deoxynucleotides, which is flanked on both sides (5′ and 3′) by “wings”. The wings are composed of 2′-O-(2-methoxyethyl) nucleotides, also known as 2′-MOE nucleotides. The “motif” of each gapmer oligonucleotide is illustrated in Table 2 and indicates the number of nucleotides in each gap region and wing, for example, “5-10-5” indicates a gapmer having a 10-nucleotide gap region flanked by 5-nucleotide wings. Similarly, the motif “5-9-6” indicates a 9-nucleotide gap region flanked by 5-nucleotide wing on the 5′ side and a 6-nucleotide wing on the 3′ side. ISIS 29848 is a mixture of randomized oligomeric compound; its sequence is shown in Table 2, where N can be A, T, C or G. The internucleoside (backbone) linkages are phosphorothioate throughout the oligonucleotides in Table 2. Unmodified cytosines are indicated by “C” in the nucleotide sequence; all other cytosines are 5-methylcytosines.

TABLE 2

Control oligonucleotides for cell line testing,
oligomeric compound screening and phenotypic assays

| | | | | | |
|---|---|---|---|---|---|
| | Target | Species of | | | SEQ ID |
| ISIS # | Name | Target | Sequence 5′ to 3′) | Motif | NO |

| | | | | | |
|---|---|---|---|---|---|
| 113131 | CD86 | Human | CGTGTGTCTGTGCTAGTCCC | 5-10-5 | 19 |
| 289865 | forkhead box | Human | GGCAACGTGAACAGGTCCAA | 5-10-5 | 20 |
| | O1A | | | | |
| | (rhabdomyos | | | | |
| | arcoma) | | | | |
| 25237 | integrin beta | Human | GCCCAATGCTGGACATGC | 4-10-4 | 21 |
| | 3 | | | | |
| 196103 | integrin beta | Human | AGCCCATTGCTGGACATGCA | 5-10-5 | 22 |
| | 3 | | | | |
| 148715 | Jagged 2 | Human; | TTGTCCCAGTCCCAGGCCTC | 5-10-5 | 23 |
| | | Mouse; | | | |
| | | Rat | | | |
| 18076 | Jun N- | Human | CTTC^{uC}GTTGGA^{u}C^{u}CCCTGGG | 5-9-6 | 24 |
| | Tenninal | | | | |
| | Kinase-1 | | | | |
| 18078 | Jun N- | Human | GTGCG^{u}CG^{u}CGAG^{u}C^{u}C^{u}CGAAATC | 5-9-6 | 25 |
| | Terminal | | | | |
| | Kinase-2 | | | | |
| 183881 | kinesin-like 1 | Human | ATCCAAGTGCTACTGTAGTA | 5-10-5 | 26 |
| 29848 | none | none | NNNNNNNNNNNNNNNNNNNN | 5-10-5 | 27 |
| 22687 | Notch | Human; | GCCCTCCATGCTGGCACAGG | 5-10-5 | 28 |
| | (Drosophila) | Mouse | | | |
| | homolog 1 | | | | |
| 105990 | Peroxisome | Human | AGCAAAAGATCAATCCGTTA | 5-10-5 | 29 |
| | proliferator | | | | |
| | activated | | | | |
| | receptor | | | | |
| | gamma | | | | |
| 336806 | Raf kinase C | Human | TACAGAAGGCTGGGCCTTGA | 5-10-5 | 30 |
| 15770 | Raf kinase C | Mouse; | ATGCATT^{u}CTG^{u}C^{u}C^{u}C^{u}C^{u}CAAGGA | 5-10-5 | 31 |
| | | Murine | | | |
| | | sarcoma | | | |
| | | virus; Rat | | | |

The concentration of oligonucleotide used varies from cell line to cell line. To determine the optimal oligonucleotide concentration for a particular cell line, the cells are treated with a positive control oligonucleotide at a range of concentrations. Positive controls are shown in Table 2. For human and non-human primate cells, the positive control oligonucleotide is ISIS 18078. For mouse or rat cells the positive control oligonucleotide is ISIS 15770. The concentration of positive control oligonucleotide that results in 80% inhibition of the target mRNA, for example, human Jun N-terminal Kinase 2 for ISIS 18078, is then utilized as the screening concentration for new oligonucleotides in subsequent experiments for that cell line. If 80% inhibition is not achieved, the lowest concentration of positive control oligonucleotide that results in 60% inhibition of the target mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM when the antisense oligonucleotide is transfected using a liposome reagent and 1 μM to 40 μM when the antisense oligonucleotide is transfected by electroporation.

### Example 2

### Real-Time Quantitative PCR Analysis of ChREBP mRNA Levels

Quantitation of ChREBP mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

Presented in Table 3 are primers and probes used to measure GAPDH expression in the cell types described herein. The GAPDH PCR probes have JOE covalently linked to the 5′ end and TAMRA or MGB covalently linked to the 3′ end, where JOE is the fluorescent reporter dye and TAMRA or MGB is the quencher dye. In some cell types, primers and probe designed to a GAPDH sequence from a different species are used to measure GAPDH expression. For example, a human GAPDH primer and probe set is used to measure GAPDH expression in monkey-derived cells and cell lines.

TABLE 3
GAPDH primers and probes for use in real-time PCR

| Target | | Sequence | | SEQ ID |
|---|---|---|---|---|
| Name | Species | Description | Sequence (5′ to 3′) | NO |
| GAPDH | Human | Forward Primer | CAACGGATTTGGTCGTATTGG | 32 |
| GAPDH | Human | Reverse Primer | GGCAACAATATCCACTTTACCAGAGT | 33 |
| GAPDH | Human | Probe | CGCCTGGTCACCAGGGCTGCT | 34 |
| GAPDH | Human | Forward Primer | GAAGGTGAAGGTCGGAGTC | 35 |
| GAPDH | Human | Reverse Primer | GAAGATGGTGATGGGATTTC | 36 |
| GAPDH | Human | Probe | CAAGCTTCCCGTTCTCAGCC | 37 |
| GAPDH | Human | Forward Primer | GAAGGTGAAGGTCGGAGTC | 35 |
| GAPDH | Human | Reverse Primer | GAAGATGGTGATGGGATTTC | 36 |
| GAPDH | Human | Probe | TGGAATCATATTGGAACATG | 38 |
| GAPDH | Mouse | Forward Primer | GGCAAATTCAACGGCACAGT | 39 |
| GAPDH | Mouse | Reverse Primer | GGGTCTCGCTCCTGGAAGAT | 40 |
| GAPDH | Mouse | Probe | AAGGCCGAGAATGGGAAGCTTGTCATC | 41 |
| GAPDH | Rat | Forward Primer | TGTTCTAGAGACAGCCGCATCTT | 42 |
| GAPDH | Rat | Reverse Primer | CACCGACCTTCACCATCTTGT | 43 |
| GAPDH | Rat | Probe | TTGTGCAGTGCCAGCCTCGTCTCA | 44 |

### Example 3

### Antisense Inhibition of Human ChREBP Expression by Oligomeric Compounds

A series of oligomeric compounds was designed to target different regions of human ChREBP, using published sequences cited in Table 1. The compounds are shown in Table 4a. All compounds in Table 4a are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the following primer-probe set designed to hybridize to human ChREBP:

Forward primer: CACCGTGACCTTGGGTGACT
(incorporated herein as SEQ ID NO: 45)
Reverse primer: CATCCCCATTTTGCAGATTGA
(incorporated herein as SEQ ID NO: 46)

And the PCR probe was:

FAM-TCCGCTGTCTTTGGACCGCTGTGT-TAMRA
(incorporated herein as SEQ ID NO: 47),

where FAM is the fluorescent dye and TAMRA is the quencher dye.

Data are averages from two experiments in which HepG2 cells were treated with 150 nM of the disclosed oligomeric compounds using LIPOFECTIN™. A reduction in expression is expressed as percent inhibition in Table 4a. The control oligomeric compound used was SEQ ID NO: 25. These empirical results were then evaluated and active target segments were determined. In this example, active target segments were identified as being those segments of the target nucleic acid wherein at least two active antisense compounds are shown to hybridize within the segment and reduce expression of the target nucleic acid by at least 45%. One skilled in the art will understand that the percent inhibition disclosed herein will vary in subsequent studies based on numerous assay-to-assay factors. Preferably, the at least two active antisense compounds are separated along the target nucleic acid by about 60 nucleobases, more preferably by about 30 nucleobases, still more preferably are contiguous and most preferably they overlap.

TABLE 4a

Inhibition of human ChREBP mRNA levels by
chimeric oligonucleotides having 2′-MOE
wings and deoxy gap

| | | | | | |
|---|---|---|---|---|---|
| | Target | Target | | % | SEQ |
| ISIS # | SEQ ID NO | Site | Sequence (5′ to 3′) | Inhibitiou | ID NO |

| | | | | | |
|---|---|---|---|---|---|
| 354601 | 1 | 2356 | GGCAAGACTGTCACCCCCTC | 62 | 51 |
| 354602 | 2 | 411 | ACGCTGTGGCCACACGGTGG | 58 | 52 |
| 354600 | 3 | 448 | GGTAAAGAAATACTGGATAT | 0 | 53 |
| 354603 | 4 | 104 | TTCACTGCCTGTGGTAGGGA | 11 | 54 |
| 354543 | 5 | 7 | TGGTCCCTGCTCCGCGCAGC | 28 | 55 |
| 354544 | 5 | 79 | ACCCGCGGGACCTGCAAGCC | 45 | 56 |
| 354545 | 5 | 130 | AGACTCGGGTCCTCCGAGTC | 0 | 57 |
| 354546 | 5 | 312 | GGCTCAAGCACTCGAAGAGG | 39 | 58 |
| 354547 | 5 | 376 | CAGAGCAGCTTGAGGCCTTT | 55 | 59 |
| 354548 | 5 | 439 | CGCTTCACATACTGGATATA | 0 | 60 |
| 354549 | 5 | 469 | GTCACGAAGCCACACACGGG | 34 | 61 |
| 354550 | 5 | 604 | AGCCGCTTCTTGTAGTAGAT | 50 | 62 |
| 354551 | 5 | 609 | TACGGAGCCGCTTCTTGTAG | 17 | 63 |
| 354552 | 5 | 630 | GGTCATCTTCCCTGCTGGGC | 40 | 64 |
| 354553 | 5 | 709 | GGGACCACACTGGAGAAGAG | 42 | 65 |
| 354554 | 5 | 757 | AGGAGCTGCCGCCCACCGGG | 59 | 66 |
| 354555 | 5 | 793 | GTGTCTGAGATGTCGGACAA | 13 | 67 |
| 354556 | 5 | 820 | GGGCCGGACTGAGTCATGGT | 62 | 68 |
| 354557 | 5 | 852 | AGGCATCCTCAGGCGGCAGC | 54 | 69 |
| 354558 | 5 | 861 | TGCCGACGTAGGCATCCTCA | 9 | 70 |
| 354559 | 5 | 922 | TCCATGAAGTCATCCAGGCT | 47 | 71 |
| 354560 | 5 | 1029 | CACTGCTGAAGAGGGAGTCA | 8 | 72 |
| 354561 | 5 | 1094 | ACGGCTGTGTCCAGAGAGGT | 61 | 73 |
| 354562 | 5 | 1325 | AGAGAAGAGAGGCTCTTCCT | 45 | 74 |
| 354563 | 5 | 1629 | CTGTGAGCAGCTGTGTGAGG | 36 | 75 |
| 354564 | 5 | 1880 | GAGGTCCCCTGACAGCCGCC | 52 | 76 |
| 354565 | 5 | 1904 | AGTCCCAGGGCCTGGCATGG | 58 | 77 |
| 354566 | 5 | 1999 | GCGGAGATGTGTGTGATACG | 11 | 78 |
| 354567 | 5 | 2004 | GCTCCGCGGAGATGTGTGTG | 21 | 79 |
| 354568 | 5 | 2036 | AAACCCCAGCTTGATGTTGA | 16 | 80 |
| 233333 | 5 | 2091 | TCACCTTGAGGCTGGGCTGG | 49 | 81 |
| 233334 | 5 | 2096 | TTTGCTCACCTTGAGGCTGG | 46 | 82 |
| 354569 | 5 | 2103 | TGGTAGCTTTGCTCACCTTG | 19 | 83 |
| 354570 | 5 | 2121 | CAGCTGTCTTCTGCAGCGTG | 30 | 84 |
| 354571 | 5 | 2297 | GTCATCAAACATGTCTCGCA | 5 | 85 |
| 354572 | 5 | 2395 | GCCGTGGACACCATCCCGTT | 66 | 86 |
| 354573 | 5 | 2499 | TGCCCAGCTGGCGTAGGGAG | 35 | 87 |
| 354574 | 5 | 2527 | CCCGGGTCGGTCAGGATACT | 52 | 88 |
| 354575 | 5 | 2586 | ATAAAGGTTTGCCAAGGGTG | 44 | 89 |
| 354576 | 5 | 2597 | TGGCCAGGACTATAAAGGTT | 62 | 90 |
| 354577 | 5 | 2669 | GCCCAGAGATGATCCCTGGA | 35 | 91 |
| 354578 | 5 | 2675 | GGGAGTGCCCAGAGATGATC | 50 | 92 |
| 354579 | 5 | 2759 | TTGACCTCCAGGAGGTGGCA | 52 | 93 |
| 354580 | 5 | 2774 | GGGACTCTGCTCTTCTTGAC | 46 | 94 |
| 354581 | 5 | 2826 | GGACGAGTCACCCAAGGTCA | 69 | 95 |
| 354582 | 5 | 2866 | CCCCATTTTGCAGATTGAAA | 20 | 96 |
| 354583 | 5 | 2889 | TCTGCTGATTGAACCTTCCC | 49 | 97 |
| 354584 | 5 | 2895 | GGGTCATCTGGTGATTGAAC | 37 | 98 |
| 354585 | 5 | 2983 | GACAGATAAACAGCATCCTC | 15 | 99 |
| 354586 | 5 | 3021 | GAGGTCTGTGCCCCACCTGT | 66 | 100 |
| 354587 | 5 | 3045 | TTTCTGCTTCTCTGCTCAGG | 52 | 101 |
| 354588 | 5 | 3062 | GAGAGAGGGAACCTCCTTTT | 61 | 102 |
| 354589 | 5 | 3077 | AGCAGTGAAGGAGCAGAGAG | 22 | 103 |
| 354590 | 5 | 3141 | GTCTCCTGGGATCAGGCCCT | 64 | 104 |
| 354591 | 5 | 3168 | CCCTGCTGTGGTCACTCTGG | 71 | 105 |
| 354592 | 5 | 3243 | TTGCTTTTATTGGTCAAGAA | 51 | 106 |
| 233313 | 6 | 564 | GCATCACCACCTCGATGCGC | 42 | 107 |
| 233321 | 6 | 877 | TGGATCATGTCAGCATTGCC | 47 | 108 |
| 354593 | 6 | 2097 | CCGCACGCTCCTTGAGGCTG | 21 | 109 |
| 233345 | 6 | 2275 | AACTTGCAGTTGTGCAGCGT | 41 | 110 |
| 233346 | 6 | 2280 | CCCAGAACTTCCAGTTGTGC | 29 | 111 |
| 233347 | 6 | 2289 | TGCTGAACACCCAGAACTTC | 27 | 112 |
| 233348 | 6 | 2294 | GAGGATGCTGAACACCCAGA | 25 | 113 |
| 233349 | 6 | 2299 | CGGATGAGGATGCTGAACAC | 54 | 114 |
| 233352 | 6 | 2374 | CAGGCCAGTGAGGTCTGGCG | 20 | 115 |
| 233353 | 6 | 2379 | CCAGCCAGGCCAGTGAGGTC | 58 | 116 |
| 233354 | 6 | 2419 | AGGACAGTTGGCCGGAGAGC | 25 | 117 |
| 233356 | 6 | 2497 | GCCCGTGTGGCTTGCTCAGG | 59 | 118 |
| 233357 | 6 | 2502 | TGACTGCCCGTGTGGCTTGC | 32 | 119 |
| 233358 | 6 | 2507 | CTCTGTGACTGCCCGTGTGG | 51 | 120 |
| 233359 | 6 | 2512 | GTGCCCTCTGTGACTGCCCG | 56 | 121 |
| 233367 | 6 | 3197 | GCAGACAGTTTTTGCTTTTA | 5 | 122 |
| 354594 | 7 | 1971 | GGTTCTTGTTGCTGTCTGGA | 40 | 123 |
| 354595 | 9 | 1719 | GACAGCCGCCGTTCACTGCC | 22 | 124 |
| 354596 | 10 | 7456 | CAGATCCCAAAGGAAGGCCG | 24 | 125 |
| 354597 | 10 | 18820 | TGCCTTCCGCCTAGGGAGAC | 4 | 126 |
| 354598 | 10 | 25206 | GCAACAGCAGTTAGGGCCAG | 13 | 127 |

Table 4b shows the start nucleotide and stop nucleotide positions on SEQ ID NO 5 for the above antisense compounds. Similarly, table 4c shows the start and stop nucleotide positions on SEQ ID NO: 1 for the above antisense compounds. One of ordinary skill in the art will readily determine the start and stop nucleotide positions on the other target gene sequences discussed in Table 1.

TABLE 4b
SEQ ID NO 5

| | Start | Stop |
|---|---|---|
| Isis # | Nucleotide | Nucleotide |
| 354543 | 7 | 26 |
| 354544 | 79 | 98 |
| 354545 | 130 | 149 |
| 354546 | 312 | 331 |
| 354547 | 376 | 395 |
| 354548 | 439 | 458 |
| 354549 | 469 | 488 |
| 233313 | 564 | 583 |
| 354550 | 604 | 623 |
| 354551 | 609 | 628 |
| 354552 | 630 | 649 |
| 354553 | 709 | 728 |
| 354554 | 757 | 776 |
| 354555 | 793 | 812 |
| 354556 | 820 | 839 |
| 354557 | 852 | 871 |
| 354558 | 861 | 880 |
| 233321 | 877 | 896 |
| 354559 | 922 | 941 |
| 354560 | 1029 | 1048 |
| 354561 | 1094 | 1113 |
| 354562 | 1325 | 1344 |
| 354563 | 1629 | 1648 |
| 354595 | 1870 | 1889 |
| 354564 | 1880 | 1899 |
| 354565 | 1904 | 1923 |
| 354566 | 1999 | 2018 |
| 354567 | 2004 | 2023 |
| 354568 | 2036 | 2055 |
| 233333 | 2091 | 2110 |
| 233334 | 2096 | 2115 |
| 354569 | 2103 | 2122 |
| 354570 | 2121 | 2140 |
| 354571 | 2297 | 2316 |
| 233345 | 2332 | 2351 |
| 233346 | 2337 | 2356 |
| 233347 | 2346 | 2365 |
| 233348 | 2351 | 2370 |
| 233349 | 2356 | 2375 |
| 354572 | 2395 | 2414 |
| 233352 | 2431 | 2450 |
| 233353 | 2436 | 2455 |
| 233354 | 2476 | 2495 |
| 354573 | 2499 | 2518 |
| 354574 | 2527 | 2546 |
| 233356 | 2554 | 2573 |
| 233357 | 2559 | 2578 |
| 233358 | 2564 | 2583 |
| 233359 | 2569 | 2588 |
| 354575 | 2586 | 2605 |
| 354576 | 2597 | 2616 |
| 354577 | 2669 | 2688 |
| 354578 | 2675 | 2694 |
| 354579 | 2759 | 2778 |
| 354580 | 2774 | 2793 |
| 354581 | 2826 | 2845 |
| 354582 | 2866 | 2885 |
| 354583 | 2889 | 2908 |
| 354584 | 2895 | 2914 |
| 354585 | 2983 | 3002 |
| 354586 | 3021 | 3040 |
| 354587 | 3045 | 3064 |
| 354588 | 3062 | 3081 |
| 354589 | 3077 | 3096 |
| 354590 | 3141 | 3160 |
| 354591 | 3168 | 3187 |
| 354592 | 3243 | 3262 |
| 233367 | 3254 | 3273 |

TABLE 4c
SEQ ID NO 1

| | Start | Stop |
|---|---|---|
| Isis # | Nucleotide | Nucleotide |
| 354544 | 53 | 72 |
| 354545 | 104 | 123 |
| 354546 | 286 | 305 |
| 354547 | 350 | 369 |
| 354548 | 413 | 432 |
| 354549 | 443 | 462 |
| 354557 | 547 | 566 |
| 354558 | 556 | 575 |
| 233321 | 572 | 591 |
| 354559 | 617 | 636 |
| 354560 | 724 | 743 |
| 354561 | 789 | 808 |
| 354562 | 1020 | 1039 |
| 354563 | 1324 | 1343 |
| 354595 | 1562 | 1581 |
| 354564 | 1572 | 1591 |
| 354565 | 1596 | 1615 |
| 354566 | 1691 | 1710 |
| 354567 | 1696 | 1715 |
| 354568 | 1728 | 1747 |
| 233333 | 1783 | 1802 |
| 233334 | 1788 | 1807 |
| 354569 | 1795 | 1814 |
| 354570 | 1813 | 1832 |
| 354571 | 1989 | 2008 |
| 233345 | 2024 | 2043 |
| 233346 | 2029 | 2048 |
| 233347 | 2038 | 2057 |
| 233348 | 2043 | 2062 |
| 233349 | 2048 | 2067 |
| 354572 | 2087 | 2106 |
| 233352 | 2123 | 2142 |
| 233353 | 2128 | 2147 |
| 354601 | 2356 | 2375 |
| 354573 | 2481 | 2500 |
| 354574 | 2509 | 2528 |
| 233356 | 2536 | 2555 |
| 233357 | 2541 | 2560 |
| 233358 | 2546 | 2565 |
| 233359 | 2551 | 2570 |
| 354575 | 2568 | 2587 |
| 354576 | 2579 | 2598 |
| 354577 | 2651 | 2670 |
| 354578 | 2657 | 2676 |
| 354579 | 2741 | 2760 |
| 354580 | 2756 | 2775 |
| 354581 | 2808 | 2827 |
| 354582 | 2848 | 2867 |
| 354583 | 2871 | 2890 |
| 354584 | 2877 | 2896 |
| 354586 | 3003 | 3022 |
| 354587 | 3027 | 3046 |
| 354588 | 3044 | 3063 |
| 354589 | 3059 | 3078 |
| 354590 | 3123 | 3142 |
| 354591 | 3150 | 3169 |
| 354592 | 3225 | 3244 |

As stated above, antisense oligonucleotides directed to a target or more preferably to an active target segment can be from about 13 to about 80 linked nucleobases. The following Table 4d provides a non-limiting example of such antisense oligonucleotides targeting SEQ ID NO 5.

TABLE 4d

Antisense Oligonucleotides from
about 13 to about 35 Nucleobases

| | |
|---|---|
| Sequence | Length |

| | |
|---|---|
| GAGGTCTGTGCCCCACCTGT | 20 nucleobases (SEQ ID NO: 100) |
| GAGGTCTGTGCCCCA | 15 nucleobases (SEQ ID NO: 190) |
| GTCTGTGCCCCACCT | 15 nucleobases (SEQ ID NO: 191) |
| AGGTCTGTGCCCC | 13 nucleobases (SEQ ID NO: 192) |
| GAGGTCTGTGCCCCACCTGTCGGG | 24 nucleobases (SEQ ID NO: 193) |
| CCCCACCTGTCGGG | 14 nucleobases (SEQ ID NO: 194) |
| AACAGAGGTCTGTGCCCCACCTGTCGGGGAGCAAG | 35 nucleobases (SEQ ID NO: 195) |
| AGGTCTGTGCCCCACCTGTCGGGGAGCA | 27 nucleobases (SEQ ID NO: 196) |
| TGCCCCACCTGTCGGGGAGCAAG | 22 nucleobases (SEQ ID NO: 197) |

Antisense oligonucleotides directed to a target or more preferably to an active target segment can also contain mismatched nucleobases when compared to the target sequence. The following Table 4e provides a non-limiting example of such antisense oligonucleotides targeting nucleobases 2579 to 2598 of SEQ ID NO 1. Mismatched nucleobases are underlined.

TABLE 4e

Antisense Oligonucleotides from about 1-3
Nucleobases Mismatched to the Target Sequence

| | Number of mismatches to |
|---|---|
| Sequence | SEQ ID NO: 1 |
| TGGCCAGGACTATAAAGGTT | None |
| | (SEQ ID NO: 90) |
| TGGCCAGCACTATAAAGGTT | One mismatch |
| | (SEQ ID NO: 198) |
| TGGCCAGGACTATACAGGTT | One mismatch |
| | (SEQ ID NO: 199) |
| TGGCCAGGACTATAAAGAGT | Two mismatches |
| | (SEQ ID NO: 200) |
| AGGCCAGGAATATAAAGGTT | Two mismatches |
| | (SEQ ID NO: 201) |
| TAGCCAGGATTATCAAGGTT | Three mismatches |
| | (SEQ ID NO: 202) |

Active target segments were determined for SEQ ID NO: 5 using the above results. Active target segment A is nucleotides 3021 to 3187. The active antisense compounds in this segment inhibited the target nucleic acid by an average of 56% and by an average of 63% if compounds inhibiting less than 50% are removed. Active target segment B is nucleotides 3021 to 3262. The active antisense compounds in this segment inhibited the target nucleic acid by an average of 55% and by an average of 61% if compounds inhibiting less than 50% are removed. Active target segment C is nucleotides 2527 to 2616. The active antisense compounds in this segment inhibited the target nucleic acid by an average of 51%, and by an average of 56% if compounds inhibiting less than 50% are removed. Active target segment D is nucleotides 2356 to 2455. The active antisense compounds in this segment inhibited the target nucleic acid by an average of 49% and by an average of 59% if compounds inhibiting less than 50% are removed. Active target segment E is nucleotides 2356 to 2661. The active antisense compounds in this segment inhibited the target nucleic acid by an average of 47% and by an average of 57% if compounds inhibiting less than 50% are removed. Active target segment F is nucleotides 757 to 871. The active antisense compounds in this segment inhibited the target nucleic acid by an average of 47% and by an average of 58% if compounds inhibiting less than 50% are removed.

Active target segments were determined for SEQ ID NO: 1 using the above results. Active target segment AA is nucleotides 3123 to 3244. The active antisense compounds in this segment inhibited the target nucleic acid by an average of 62% and by an average of 67% if compounds inhibiting less than 60% are removed. Active target segment AB is nucleotides 3003 to 3063. The active antisense compounds in this segment inhibited the target nucleic acid by an average of 60% and by an average of 64% if compounds inhibiting less than 60% are removed. Active target segment AC is nucleotides 3003 to 3244. The active antisense compounds in this segment inhibited the target nucleic acid by an average of 55% and by an average of 61% if compounds inhibiting less than 50% are removed. Active target segment AD is nucleotides 2509 to 2598. The active antisense compounds in this segment inhibited the target nucleic acid by an average of 51% and by an average of 56% if compounds inhibiting less than 50% are removed. Active target segment AE is nucleotides 2356 to 2598. The active antisense compounds in this segment inhibited the target nucleic acid by an average of 50% and by an average of 57% if compounds inhibiting less than 50% are removed. Active target segment AF is nucleotides 2048 to 2147. The active antisense compounds in this segment inhibited the target nucleic acid by an average of 49% and by an average of 59% if compounds inhibiting less than 50% are removed.

### Example 4

### Antisense Inhibition of Mouse ChREBP Expression by Oligomeric Compounds

A series of oligomeric compounds was designed to target different regions of mouse ChREBP, using published sequences cited in Table 1. The compounds are shown in Table 5. All compounds in Table 5 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. The compounds were analyzed for their effect on gene target mRNA levels by quantitative real-time PCR as described in other examples herein, using the following primer-probe set designed to hybridize to mouse ChREBP:

Forward primer: CGGGACATGTTTGATGACTATGTC
(incorporated herein as SEQ ID NO: 48)
Reverse primer: TCGGATGAGGATGCTGAACAC
(incorporated herein as SEQ ID NO: 49)

And the PCR probe was:

FAM-CACGCTGCACAACTGGAAGTTCTG-TAMRA
(incorporated herein as SEQ ID NO: 50),

where FAM is the fluorescent dye and TAMRA is the quencher dye.

Data are averages from two experiments in which undifferentiated 3T3-L1 cells were treated with 150 nM of the disclosed oligomeric compounds using LIPOFECTIN™. A reduction in expression is expressed as percent inhibition in Table 5. The control oligomeric compound used was SEQ ID NO: 25. The target regions to which these oligomeric compounds are inhibitory are herein referred to as “active target segments.”

TABLE 5

Inhibition of mouse ChREBP mRNA levels by
chimeric oligonucleotides having 2′-MOE
wings and deoxy gap

| | | | | | |
|---|---|---|---|---|---|
| | Target | Target | | % | SEQ ID |
| ISIS # | SEQ ID NO | Site | Sequence (5′ to 3′) | Inhibition | NO |

| | | | | | |
|---|---|---|---|---|---|
| 233306 | 12 | 22 | TCACGGATAGATCCGCCAGC | 94 | 129 |
| 233307 | 12 | 152 | ACCATGAAGTGTCCGCTGTG | 97 | 130 |
| 233308 | 12 | 317 | AAGTTCTTCCACTTGGGAGA | 73 | 131 |
| 233309 | 12 | 337 | ATAGCAACTTGAGGCCTTTG | 87 | 132 |
| 233310 | 12 | 386 | ATGTACCAGGCTCTCCAGAT | 63 | 133 |
| 233311 | 12 | 406 | TCCTCCGTTGCACATACTGA | 86 | 134 |
| 233312 | 12 | 472 | CAGGTTTCCGGTGCTCATCT | 63 | 135 |
| 233313 | 12 | 526 | GCATCACCACCTCGATGCGC | 77 | 107 |
| 233314 | 12 | 574 | ACTTACGGAGCCGCTTTTTG | 62 | 136 |
| 233315 | 12 | 598 | CCAGGAAATGCCCTTCCCTG | 88 | 137 |
| 233316 | 12 | 618 | ACCTTCCACCTGCTTGGGAG | 89 | 138 |
| 233317 | 12 | 667 | CCACGCTGGAGAAGAGCTGT | 55 | 139 |
| 233318 | 12 | 717 | AAGCTGCCGGCCCCCAGGCT | 37 | 140 |
| 233319 | 12 | 759 | GAGTGTGTCGGAGATATCGG | 96 | 141 |
| 233320 | 12 | 779 | CTGGGCTGTGTCATGGTGAA | 92 | 142 |
| 233321 | 12 | 839 | TGGATCATGTCAGCATTGCC | 0 | 108 |
| 233322 | 12 | 895 | AATCTGATATCTCCATGAAG | 94 | 143 |
| 233323 | 12 | 1087 | TGTTCCGAGCCTGTAGGCGT | 25 | 144 |
| 233324 | 12 | 1146 | TTCAGGAAGAAGGAATTCAG | 87 | 145 |
| 233325 | 12 | 1235 | CAGGGCTCTAAGCCATGCAC | 69 | 146 |
| 233326 | 12 | 1622 | GCCCTGAGCAGCTGTGTAAG | 83 | 147 |
| 233327 | 12 | 1859 | CGCTCACTGCGGCTGGAGGC | 68 | 148 |
| 233328 | 12 | 1942 | GGCTTAGGACAGTTTGAGGG | 89 | 149 |
| 233329 | 12 | 1952 | ACACGACCCCGGCTTAGGAC | 84 | 150 |
| 233330 | 12 | 1962 | GTTGTTGTCTACACGACCCC | 90 | 151 |
| 233331 | 12 | 2033 | AATCCTAGCTTAATATTGAA | 90 | 152 |
| 233332 | 12 | 2065 | TGAGCGTGCTGACAAGTCGG | 91 | 153 |
| 233333 | 12 | 2089 | TCACCTTGAGGCTGGGCTGG | 91 | 81 |
| 233334 | 12 | 2094 | TTTGCTCACCTTGAGGCTGG | 87 | 82 |
| 233335 | 12 | 2104 | GCGTGGTTGCTTTGCTCACC | 85 | 154 |
| 233336 | 12 | 2114 | GTCTTCTGAAGCGTGGTTGC | 89 | 155 |
| 233337 | 12 | 2199 | GAGCTCCTCTATTTCATCCC | 78 | 156 |
| 233338 | 12 | 2227 | GCTGCTGGCACAAGTTGATG | 80 | 157 |
| 233339 | 12 | 2237 | GCCGGTAGCTGCTGCTGGCA | 79 | 158 |
| 233340 | 12 | 2247 | CACCCCGGTGGCCGGTAGCT | 13 | 159 |
| 233341 | 12 | 2257 | GTGTGATGGGCACCCCGGTG | 81 | 160 |
| 233342 | 12 | 2281 | GCCGCATCTGGTCAAAGCGC | 80 | 161 |
| 233343 | 12 | 2291 | TCAAACATGTCCCGCATCTG | 76 | 162 |
| 233344 | 12 | 2301 | GACATAGTCATCAAACATGT | 50 | 163 |
| 233345 | 12 | 2330 | AACTTCCAGTTGTGCAGCGT | 93 | 110 |
| 233346 | 12 | 2335 | CCCAGAACTTCCAGTTGTGC | 0 | 111 |
| 233347 | 12 | 2344 | TGCTGAACACCCAGAACTTC | 84 | 112 |
| 233348 | 12 | 2349 | GAGGATGCTGAACACCCAGA | 81 | 113 |
| 233349 | 12 | 2354 | CGGATGAGGATGCTGAACAC | 64 | 114 |
| 233350 | 12 | 2387 | GACACCATCCCATTGAAGGA | 67 | 164 |
| 233351 | 12 | 2414 | TGGCGGAGGCTGTGCAAGCT | 75 | 165 |
| 233352 | 12 | 2429 | CAGGCCAGTGAGGTCTGGCG | 84 | 115 |
| 233353 | 12 | 2434 | CCAGCCAGGCCAGTGAGGTC | 91 | 116 |
| 233354 | 12 | 2474 | AGGACAGTTGGCCGGAGAGC | 92 | 117 |
| 233355 | 12 | 2486 | CGAAGGGAATTCAGGACAGT | 87 | 166 |
| 233356 | 12 | 2552 | GCCCGTGTGGCTTGCTCAGG | 90 | 118 |
| 233357 | 12 | 2557 | TGACTGCCCGTGTGGCTTGC | 85 | 119 |
| 233358 | 12 | 2562 | CTCTGTGACTGCCCGTGTGG | 88 | 120 |
| 233359 | 12 | 2567 | GTGCCCTCTGTGACTGCCCG | 82 | 121 |
| 233360 | 12 | 2594 | CACCAGGATTATAATGGTCT | 41 | 167 |
| 233361 | 12 | 2617 | TGTTCCTGGAGCTTGGAAAC | 79 | 168 |
| 233362 | 12 | 2622 | CAAGTTGTTCCTGGAGCTTG | 94 | 169 |
| 233363 | 12 | 2862 | ATCTCTCATCAGAGCTCCTG | 84 | 170 |
| 233364 | 12 | 3080 | CTACATTCATGCAAGATGCC | 83 | 171 |
| 233365 | 12 | 3085 | CACATCTACATTCATGCAAG | 84 | 172 |
| 233366 | 12 | 3140 | TTGCTTTTATTGATGAAGAA | 83 | 173 |
| 233367 | 12 | 3151 | GCAGACAGTTTTTGCTTTTA | 49 | 122 |
| 233368 | 13 | 1634 | ATGAGGACCACTGCCCTGAG | 81 | 174 |
| 233369 | 13 | 1664 | GCTCAGGCTTGGCTGGGTAC | 90 | 175 |
| 233370 | 15 | 2095 | TTGGCAAGCCCTTGAGGCTG | 92 | 176 |
| 233371 | 16 | 2095 | GCCCTGGGAGCTTGAGGCTG | 88 | 177 |
| 233372 | 16 | 2115 | GTGGGCCTCTGTGTTGGCAA | 43 | 178 |
| 233373 | 17 | 186 | TCTAAGGGAGTGTGCATTGC | 84 | 179 |
| 233296 | 18 | 4359 | TTAGCCATCCAGAAAGTCAG | 83 | 180 |
| 233297 | 18 | 7828 | AGACTGACGGTGCTGGGTAG | 79 | 181 |
| 233298 | 18 | 11169 | GGAGCCACTGCGTGGATGCT | 78 | 182 |
| 233299 | 18 | 12765 | AAAGAGAAGAGGAGCTGGAG | 87 | 183 |
| 233300 | 18 | 14919 | CTCCAGCTACCTCAGGTTTC | 78 | 184 |
| 233301 | 18 | 15445 | GAACACTCACCTGCTTGGGA | 80 | 185 |
| 233302 | 18 | 26411 | ATGAGGACCACTAGACTGGC | 70 | 186 |
| 233303 | 18 | 26739 | CCCTATTTACCGCTGGAGGC | 83 | 187 |
| 233304 | 18 | 27284 | TTGGCAAGCCCTGGGAGCTG | 76 | 188 |
| 233305 | 18 | 27452 | CCCAGCTTACTTGATGGCAG | 92 | 189 |

### Example 5

### Effects of Antisense Inhibition of ChREBP: In Vivo Studies in a Lean Mouse Model

C57BL/6J-Lepr ob/ob +/− heterozygote mice (Jackson Laboratory, Bar Harbor, Me.) were maintained on a standard rodent diet with a fat content of approximately 4% and were used as lean animals. Six-week old male C57BL/6J-Lepr ob/ob +/− mice were subcutaneously injected with ChREBP antisense oligonucleotide ISIS 233325 or ISIS 233342 at a dose of 50 mg/kg two times per week for 2½ weeks (five total doses). Saline-injected animals served as controls. Each treatment group was comprised of five animals. After the treatment period, mice were sacrificed and target levels were evaluated in liver. RNA isolation and target mRNA expression level quantitation were performed using RIBOGREEN™ as described by other examples herein. Results are shown in Table 6 as percent inhibition of ChREBP mRNA as compared to saline treated control.

TABLE 6
Inhibition of ChREBP expression in lean mice
treated with ChREBP antisense oligonucleotide

| Treatment | SEQ ID NO | % Inhibition |
|---|---|---|
| ISIS 233325 | 146 | 79 |
| ISIS 233342 | 161 | 88 |

As shown in Table 6, treatment of lean mice with antisense oligonucleotide to ChREBP results in a significant reduction in ChREBP expression.

The effects of target inhibition on glucose metabolism were evaluated in lean mice treated with ChREBP antisense oligonucleotides ISIS 233325 and ISIS 233342. Plasma glucose was measured at the start of treatment (Week 0) and 15 days (Week 2) after the first dose of oligonucleotide. Glucose levels were measured by routine clinical methods using a YSI glucose analyzer (YSI Scientific, Yellow Springs, Ohio). Average plasma glucose levels (in mg/dL) for each treatment group are shown in Table 7.

TABLE 7
Effect of ChREBP antisense oligonucleotide
on plasma glucose levels in lean mice

| Treatment | Week 0 (mg/dL) | Week 2 (mg/dL) |
|---|---|---|
| Saline | 177.3 | 156.8 |
| ISIS 233325 | 179.0 | 181.4 |
| ISIS 233342 | 170.6 | 181.5 |

As shown in Table 7, plasma glucose levels of lean animals remain in the normal range whether treated with saline or ChREBP antisense oligonucleotide.

Treated mice were further evaluated for body weight at the beginning of the study (Week 0), and after 1 week and 2 weeks of oligonucleotide or saline treatment. Average body weight (in grams) measured for each treatment group is shown in Table 8.

TABLE 8
Body weight of lean mice treated with
ChREBP antisense oligonucleotide

| Treatment | Week 0 (g) | Week 1 (g) | Week 2 (g) |
|---|---|---|---|
| Saline | 23.0 | 24.8 | 25.0 |
| ISIS 233325 | 23.6 | 25.3 | 26.1 |
| ISIS 233342 | 23.1 | 24.8 | 25.3 |

As shown in Table 8, no significant differences in total body weight were observed between oligonucleotide-treated and saline-treated lean animals at timepoints throughout the study.

Also measured upon termination of the study were spleen and liver weights. Significant changes in liver or spleen weight can indicate that a particular compound has toxic effects. Average liver and spleen weight (in grams) measured for each treatment group is shown in Table 9.

TABLE 9
Liver and spleen weight of lean mice treated
with ChREBP antisense oligonucleotide

| Treatment | Liver (g) | Spleen (g) |
|---|---|---|
| Saline | 1.1 | 0.06 |
| ISIS 233325 | 1.4 | 0.07 |
| ISIS 233342 | 1.3 | 0.07 |

As shown in Table 9, no significant differences in liver or spleen weight were observed between oligonucleotide-treated and saline-treated lean animals at the termination of the study.

To assess the physiological effects resulting from inhibition of target mRNA, the lean mice were further evaluated at the end of the treatment period for serum triglycerides (TRIG) and serum cholesterol (CHOL). Triglycerides and cholesterol were measured by routine clinical analyzer instruments (e.g. Olympus Clinical Analyzer, Melville, N.Y.). Average levels of CHOL and TRIG measured for each treatment group are shown in Table 10.

TABLE 10
Cholesterol and triglyceride levels of lean mice
treated with ChREBP antisense oligonucleotide

| Treatment | CHOL (mg/dL) | TRIG (mg/dL) |
|---|---|---|
| Saline | 114.4 | 131.8 |
| ISIS 233325 | 135.2 | 150.6 |
| ISIS 233342 | 92.4 | 127.8 |

Treatment with ISIS 233342 resulted in a decrease in both cholesterol and triglyceride levels of lean mice relative to saline-treated controls. Taken together, these results demonstrate that ChREBP antisense oligonucleotide treatment inhibits ChREBP expression in vivo and even in lean animals can lower levels of cholesterol and triglycerides.

### Example 6

### Effects of Antisense Inhibition of ChREBP: In Vivo Studies in ob/ob Mice

Leptin is a hormone produced by fat that regulates appetite. Deficiencies in this hormone in both humans and non-human animals leads to obesity. ob/ob mice have a mutation in the leptin gene which results in obesity and hyperglycemia. As such, these mice are a useful model for the investigation of obesity and diabetes and treatments designed to treat these diseases or conditions. In accordance with the present invention, the oligomeric compounds of the invention were tested in the ob/ob model of obesity and diabetes.

Six-week old male C57BL/6J-Lepr ob/ob mice (Jackson Laboratory, Bar Harbor, Me.) were subcutaneously injected with ChREBP antisense oligonucleotide ISIS 233325 or ISIS 233342 at a dose of 25 mg/kg two times per week for 4 weeks (eight total doses). Saline-injected animals served as controls. Each treatment group was comprised of eight animals. After the treatment period, mice were sacrificed and target levels were evaluated in liver. RNA isolation and target mRNA expression level quantitation were performed using RIBOGREEN™ as described by other examples herein. Results are shown in Table 11 as percent inhibition of ChREBP mRNA as compared to saline treated control.

TABLE 11
Inhibition of ChREBP expression in ob/ob mice
treated with ChREBP antisense oligonucleotide

| Treatment | SEQ ID NO | % Inhibition |
|---|---|---|
| ISIS 233325 | 146 | 76 |
| ISIS 233342 | 161 | 70 |

As shown in Table 11, treatment of ob/ob mice with antisense oligonucleotide to ChREBP results in a significant reduction in CHREBP expression.

The effects of target inhibition on glucose metabolism were evaluated in ob/ob mice treated with CHREBP antisense oligonucleotides ISIS 233325 and ISIS 233342. Plasma glucose was measured prior to the start of treatment (Week 0), at Week 2 and at Week 4. Glucose levels were measured by routine clinical methods using a YSI glucose analyzer (YSI Scientific, Yellow Springs, Ohio). Average plasma glucose levels (in mg/dL) for each treatment group are shown in Table 12.

TABLE 12
Effect of ChREBP antisense oligonucleotide
on plasma glucose levels in ob/ob mice

| Treatment | Week 0 (mg/dL) | Week 2 (mg/dL) | Week 4 (mg/dL) |
|---|---|---|---|
| Saline | 361 | 427 | 408 |
| ISIS 233325 | 353 | 343 | 240 |
| ISIS 233342 | 353 | 346 | 193 |

As shown in Table 12, treatment with ChREBP antisense oligonucleotide significantly reduces plasma glucose levels of ob/ob mice.

Body weight and food consumption were monitored throughout the study. Cumulative food consumption for each treatment group was similar to that of saline-treated mice. Mice were evaluated for body weight at the beginning of the study (Week 0), and after 1 week, 2 weeks and 3 weeks of oligonucleotide or saline treatment. Average body weight (in grams) measured for each treatment group is shown in Table 13.

TABLE 13
Body weight of ob/ob mice treated with
ChREBP antisense oligonucleotide

| Treatment | Week 0 (g) | Week 1 (g) | Week 2 (g) | Week 3 (g) |
|---|---|---|---|---|
| Saline | 35.4 | 42.6 | 47.0 | 49.1 |
| ISIS 233325 | 34.9 | 42.6 | 47.1 | 49.9 |
| ISIS 233342 | 34.8 | 41.7 | 45.9 | 48.8 |

Also measured upon termination of the study were spleen, liver and epididymal fat pad weights. Average liver, spleen and fat pad weight (in grams) measured for each treatment group is shown in Table 14.

TABLE 14
Liver and spleen weight of ob/ob mice treated
with ChREBP antisense oligonucleotide

| Treatment | Liver (g) | Spleen (g) | Fat Pad (g) |
|---|---|---|---|
| Saline | 3.6 | 0.08 | 3.9 |
| ISIS 233325 | 4.5 | 0.07 | 4.1 |
| ISIS 233342 | 5.0 | 0.07 | 3.7 |

To assess the effects of inhibition of target mRNA on triglyceride levels, the ob/ob mice were further evaluated at the end of the treatment period for serum triglycerides (TRIG). Triglycerides were measured by routine clinical analyzer instruments (e.g. Olympus Clinical Analyzer, Melville, N.Y.). Average levels of TRIG measured for each treatment group are shown in Table 15.

TABLE 15
Triglyceride levels of ob/ob mice treated
with ChREBP antisense oligonucleotide

| Treatment | TRIG (mg/dL) |
|---|---|
| Saline | 139 |
| ISIS 233325 | 148 |
| ISIS 233342 | 152 |

Taken together, these results demonstrate that administration of ChREBP antisense oligonucleotide to obese or diabetic animals is an effective treatment for lowering plasma glucose levels.

### Example 7

### Effects of Antisense Inhibition of ChREBP: In Vivo Studies in db/db Mice

Leptin is a hormone produced by fat that regulates appetite. Deficiencies in this hormone in both humans and non-human animals leads to obesity. db/db mice have a mutation in the leptin receptor gene which results in obesity and hyperglycemia. As such, these mice are a useful model for the investigation of obesity and diabetes and treatments designed to treat these diseases or conditions. db/db mice, which have lower circulating levels of insulin and are more hyperglycemic than ob/ob mice which harbor a mutation in the leptin gene, are often used as a rodent model of type 2 diabetes. In accordance with the present invention, oligomeric compounds of the present invention were tested in the db/db model of obesity and diabetes.

Six-week old male C57B1/6J-Lepr db/db mice (Jackson Laboratory, Bar Harbor, Me.) were subcutaneously injected with CHREBP antisense oligonucleotide ISIS 233325 at a dose of 25 mg/kg two times per week for 4 weeks (eight total doses). Saline-injected animals served as controls. Each treatment group was comprised of seven or eight animals. After the treatment period, mice were sacrificed and target levels were evaluated in liver and fat. RNA isolation and target mRNA expression level quantitation were performed using RIBOGREEN as described by other examples herein. Results are shown in Table 16 as percent inhibition of ChREBP mRNA as compared to saline treated control.

TABLE 16
% Inhibition of ChREBP expression in db/db mice
treated with ChREBP antisense oligonucleotide

| | | % Inhibition | % Inhibition |
|---|---|---|---|
| Treatment | SEQ ID NO | Liver | Fat |
| ISIS 233325 | 146 | 75 | 52 |

As shown in Table 16, treatment of db/db mice with antisense oligonucleotide to ChREBP results in a significant reduction in CHREBP expression in both liver and fat.

The effects of target inhibition on glucose metabolism were evaluated in db/db mice treated with CHREBP antisense oligonucleotide ISIS 233325. Plasma glucose was measured prior to the start of treatment (Week 0), at Week 2 and at Week 4. In addition, plasma glucose level after fasting was measured at week 3. Glucose levels were measured by routine clinical methods using a YSI glucose analyzer (YSI Scientific, Yellow Springs, Ohio). Average plasma glucose levels (in mg/dL) for each treatment group are shown in Table 17.

TABLE 17
Effect of ChREBP antisense oligonucleotide
on plasma glucose levels in db/db mice

| | Week 0 | Week 2 | Week 4 | Fasted |
|---|---|---|---|---|
| Treatment | (mg/dL) | (mg/dL) | (mg/dL) | (mg/dL) |
| Saline | 311 | 452 | 570 | 194 |
| ISIS 233325 | 310 | 376 | 468 | 161 |

As shown in Table 17, treatment of db/db mice with ChREBP antisense oligonucleotide resulted in a significant decrease in plasma glucose levels.

Body weight and food consumption were monitored throughout the study. Cumulative food consumption for each treatment group was similar to that of saline-treated mice. Mice were evaluated for body weight at the beginning of the study (Week 0), and after 1 week, 2 weeks and 3 weeks of oligonucleotide or saline treatment. Average body weight (in grams) measured for each treatment group is shown in Table 18.

TABLE 18
Body weight of db/db mice treated with
ChREBP antisense oligonucleotide

| Treatment | Week 0 (g) | Week 1 (g) | Week 2 (g) | Week 3 (g) |
|---|---|---|---|---|
| Saline | 30.8 | 33.9 | 36.7 | 39.7 |
| ISIS 233325 | 31.7 | 35.8 | 40.4 | 43.3 |

Also measured upon termination of the study were spleen, liver and epididymal fat pad weights. Average liver, spleen and fat pad weight (in grams) measured for each treatment group is shown in Table 19.

TABLE 19
Liver and spleen weight of db/db mice treated
with ChREBP antisense oligonucleotide

| Treatment | Liver (g) | Spleen (g) | Fat Pad (g) |
|---|---|---|---|
| Saline | 2.0 | 0.06 | 1.7 |
| ISIS 233325 | 2.8 | 0.06 | 1.9 |

As shown in Tables 18 and 19, no significant differences in body weight, fat pad weight or spleen weight were observed between oligonucleotide-treated and saline-treated db/db animals throughout (body weight) or at the termination (organ weight) of the study. Treatment with ISIS 233325 led to a slight increase in liver weight.

To assess the physiological effects resulting from inhibition of target mRNA, the db/db mice were further evaluated at the end of the treatment period for serum glucose (GLUC) and serum triglycerides (TRIG). Glucose and triglycerides were measured by routine clinical analyzer instruments (e.g. Olympus Clinical Analyzer, Melville, N.Y.). Average levels of GLUC and TRIG measured for each treatment group are shown in Table 20.

TABLE 20
Physiological effects of ChREBP antisense
oligonucleotide treatment

| Treatment | GLUC (mg/dL) | TRIG (mg/dL) |
|---|---|---|
| Saline | 573 | 212 |
| ISIS 233325 | 496 | 183 |

As shown in Table 20, db/db mice treated with ChREBP antisense oligonucleotide demonstrate a reduction in both serum glucose and triglyceride levels. Taken together, these results demonstrate that ChREBP antisense oligonucleotides inhibit ChREBP expression in vivo and are useful for the reduction of glucose and triglyceride levels diabetic animals.

Various modifications of the invention, in addition to those described herein, will be apparent to those skilled in the art from the foregoing description. Such modifications are also intended to fall within the scope of the appended claims. Each of the patents, applications, printed publications, and other published documents mentioned or referred to in this specification are herein incorporated by reference in their entirety. Those skilled in the art will appreciate that numerous changes and modifications may be made to the embodiments of the invention and that such changes and modifications may be made without departing from the spirit of the invention. It is therefore intended that the appended claims cover all such equivalent variations as fall within the true spirit and scope of the invention.

1. A chimeric antisense compound 13 to 50 nucleobases in length and comprising at least one chemical modification, wherein the compound is targeted to a nucleic acid molecule encoding human ChREBP.

2. The compound of claim 1 wherein said compound hybridizes within an active target segment of the nucleic acid molecule encoding ChREBP comprising, active target segment A, active target segment B, active target segment C, active target segment D, active target segment E, active target segment F, active target segment AA, active target segment AB, active target segment AC, active target segment AD, active target segment AE or active target segment AF.

3. The compound of claim 1 comprising 13 to 30 nucleobases.

4. The compound of claim 1 wherein the at least one chemical modification is selected from the group consisting of a modified internucleoside linkage, a modified nucleobase, a modified sugar moiety or combinations thereof.

5. The compound of claim 4, wherein the modified internucleoside linkage is a phosphorothioate linkage.

6. The compound of claim 4, wherein the modified nucleobase is a 5-methylcytosine.

7. The compound of claim 4, wherein the modified sugar moiety is a high affinity modification selected from the group consisting of a 2′-O-(2-methoxyethyl), a 2′-O-methyl, an LNA, an ENA or combinations thereof.

8. The compound of claim 4, wherein the chimeric oligonucleotide comprises deoxynucleotides in a first region, at least one high affinity modification in each of a second region and a third region, which flank the first region on the 5′ and 3′ ends, respectively, and at least one phosphorothioate internucleoside linkage.

9. The compound of claim 8, wherein the first region is ten deoxynucleotides in length and the second and third regions are each five nucleotides in length and each comprise five 2′-O-(2-methoxyethyl) nucleotides, and wherein each internucleoside linkage in the chimeric oligonucleotide is a phosphorothioate.

10. The compound of claim 1, wherein the compound is complementary to at least a contiguous 13 nucleobase portion an active target segment.

11. The compound of claim 1, wherein the compound hybridizes within an active target segment, the compound comprising at least 3 mismatched nucleotides to the active target segment sequence where hybridized.

12. The compound of claim 1 wherein the compound hybridizes with at least a 13 nucleotide portion of nucleotides 3168 to 3187, nucleotides 3021 to 3040 or nucleotides 3141 to 3160 of active target segment A; nucleotides 3243 to 3262 of active target segment B; nucleotides 2597 to 2616 or nucleotides 2554 to 2573 of active target segment C, nucleotides 2395 to 2414 or nucleotides 2436 to 2455 of active target segment D, nucleotides 2436 to 2455 of active target segment E, nucleotides 820 to 839 of active target segment F, nucleotides 3150 to 3196 of active target segment AA, nucleotides 3003 to 3022 of active target segment AB, nucleotides 3123 to 3142 of active target segment AC, nucleotides 2579 to 2598 of active target segment AD, nucleotides 2356 to 2375 of active target segment AE or nucleotides 2087 to 2196 of active target segment AF.

13. The compound of claim 1 used to make a pharmaceutical composition.

14. A method of inhibiting expression of ChREBP in cells, tissues or animals, comprising contacting said cells or tissues with the compound of claim 1.

15. A method of lowering plasma glucose levels in an animal, comprising administering to said animal the compound of claim 1.

16. A method of lowering triglyceride levels in an animal, comprising administering to said animal the compound of claim 1.

17. A method of lowering cholesterol levels in an animal, comprising administering to said animal the compound of claim 1.

18. The method of claim 14 wherein the animal is a primate.

19. A method of preventing, ameliorating or lessening the severity of a disease or a condition in an animal, comprising contacting said animal with an effective amount of the compound of claim 1 so that expression of ChREBP is inhibited.

20. The method of claim 19, wherein the disease or condition is obesity.

21. The method of claim 19, wherein the disease or condition is diabetes.

22. The method of claim 19, wherein the disease or condition is hyperglycemia.

23. The method of claim 19, wherein the disease or condition is hypertriglyceridemia.

24. The method of claim 19 wherein the disease or condition is metabolic syndrome X.
